(19) United States
(12) Patent Application Publication
(10) Pub. No.: US 2017/0044560 A1
(43) Pub. Date: Feb. 16, 2017

(54) COMPOSITIONS AND METHODS FOR REDUCING PATHOGEN-INDUCED CITRUS GREENING

(71) Applicant: Forrest Innovations Ltd., Caesarea (IL)

(72) Inventors: Nitzan PALDI, Moshav Bar Giora (IL); Humberto Freire BONCRISTIANI JUNIOR, Odenton, MD (US); Eyal MAORI, Rishon-LeZion (IL); Alon WELLNER, Herzlia (IL); Shaul ILAN, Nofit (IL)

(73) Assignee: Forrest Innovations Ltd., Caesarea (IL)

(21) Appl. No.: 15/306,095

(22) PCT Filed: May 4, 2015
(86) PCT No.: PCT/IL2015/050469

§ 371 Date: Oct. 23, 2016

Related U.S. Application Data
(60) Provisional application No. 61/988,234, filed on May 4, 2014.
(60) Provisional application No. 61/988,235, filed on May 4, 2014.
(60) Provisional application No. 61/988,246, filed on May 4, 2014.
(60) Provisional application No. 61/988,237, filed on May 4, 2014.
(60) Provisional application No. 61/988,236, filed on May 4, 2014.

(51) Int. Cl. C12N 15/82

(52) U.S. Cl. CPC C12N 15/8218; C12N 15/8261; C12N 15/8271; C12N 15/8243; C12N 15/8245; C12N 15/8273; C12N 15/827

(57) ABSTRACT

The present invention, in some embodiments thereof, relates to methods for enhancing fitness of pathogen-infected plants, and, more particularly, but not exclusively, to methods of using RNA interference for modulation of plant-pathogen resistance response gene expression. In particular, the present invention provides compositions and methods for enhancing host plant fitness and fruit yield and quality following Candidatus Liberibacter spp infection and, specifically, Candidatus Liberibacter spp infection in citrus plants and trees, as in Huang Long Bing.

### FIELD AND BACKGROUND OF THE INVENTION

Citrus huanglongbing (HLB), also known as “citrus greening” is possibly the most destructive disease of citrus. It is distributed throughout most citrus producing countries worldwide, where it generates substantial economic losses in heavily affected areas. The suspected causal agent of HLB is a fastidious, phloem-limited bacterium of the genus Candidatus Liberibacter. Three different bacterial species are associated with HLB in citrus: Candidatus Liberibacter asiaticus (CaLas), found in all HLB-affected countries except Africa, Candidatus Liberibacter africanus, presently restricted to Africa, and Candidatus Liberibacter americanus, currently limited to Brazil and China. Transmission of the pathogens occurs through the insect vectors Diaphorina citri Kuwayama, the Asian citrus psyllid, or Trioza erytrea Del Guercio, the African citrus psyllid, by dodder (Cuscuta sp.) and through grafting with diseased budwood.

Typical leaf symptoms observed in HLB-affected citrus plants are an asymmetric blotchy mottling of older leaves and a range of chlorotic patterns, often resembling zinc-deficiency symptoms. These are then followed by twig-dieback, reduced fruit production, premature fruit drop, reduced vigor and tree decline at advanced stages of the disease. Blockage of the translocation stream due to the plugging of sieve elements along with phloem necrosis appears to be a major factor of the disease process.

HLB affects all known citrus species and citrus relatives with little known resistance. Current management strategies are the removal of infected trees, attempts at elimination of the insect vector through use of insecticides, and nutritional applications. No known cure exists at present. Authorities worldwide have agreed that HLB is devastating the global citrus industry.

The multi-billion dollar annual citrus industry in Florida, California and outside of the US is severely threatened by the psyllid-CaLas vector-disease pathosystem. Presently, there is no cure for this disease and trees are routinely destroyed once severely infected. Moreover, no known relevant citrus cultivars are resistant to citrus greening disease.

Infection of plants with pathogens usually results in a series of defense responses such as the hypersensitive reaction, the production of reactive oxygen species, cell wall fortifications, the synthesis of pathogenesis-related proteins, and the production of phytoalexins.

Microarray technology has revealed much about the host transcriptional regulation in Candidatus Liberibacter spp infection and disease, despite the variability noted between citrus species and even cultivars, different plant tissues and different stages of the infection and disease. However, some reports indicate that the transcriptional response of citrus to Candidatus Liberibacter spp involves upregulation of plant defense-related response genes (plant defense proteins, constitutive disease resistance protein 1, defense-gene transcription regulators, etc), upregulation of sugar metabolism and starch synthesis genes and either upregulation or downregulation (depending on the study) of light reactions genes (Albrecht and Bowman, Plant Sci 2012, 185-186:118-130; Katagiri et al, Molec Plant-Microbe Interactions 2010, 23:1531-36; Anderson et al, Funct Plant Biol, 2010; 37:499-512; Bolton, Molec Plant-Microbe Interaction 2009; 22:487-97; Kim et al., Phytopathology 2009; 99:50-57; Martinelli et al, PLoS One 2012 7:e38039; Conrath, Plant Signaling and Behav 2006, 1:179-84; Jones and Dangl, Nature 2006; 444:323-329; Mafra et al, BMC Genomics 2013; 14:247). However, no cause-and-effect relationship between the disease and expression of individual genes or gene families has been elucidated to date.

Some proposed strategies for prevention and treatment of HLB include prevention of Candidatus Liberibacter spp infection, induction of tolerance to Candidatus Liberibacter, prevention of transmission of Candidatus Liberibacter by the psyllid vectors and enhancement of plant defense mechanisms.

US Patent Publication 2013025995 to Masaoka et al., 20130225456 to Figueredo, et al and U.S. Pat. No. 8,546,360 to Musson, IV, disclose chemical compositions for use as biocides for controlling bacterial infection in citrus trees, such as HLB.

Some suggested methods target the psyllid vector. US Patent Publication 20130266535 to Stelinski et al discloses the release of methyl salicylate attractants to lure psyllid HLB vectors away from the citrus crops. US Patent Publication 20130287727 to Woods et al also discloses the use of psyllid attractants for luring away, capturing and/or eliminating the psyllid vectors. U.S. Pat. No. 8,372,443 to Rouseff et al discloses the use of volatile compounds (for example, dimethyl sulfide) for repelling or killing the psyllid vectors transmitting HLB. US Patent Publication 20110119788 to Rodriguez Baixauli et al discloses the transgenic expression and release, in the citrus trees, of volatiles for repellency or resistance of the psyllid vectors and HLB bacteria.

Induction of the tree's defense response mechanism has been proposed. US Patent Publication 20140030228 to Blotsky et al discloses methods for biological control of plant pathogens, such as Candidatus Liberibacter, by application of bacteria to the trees (“priming”). US Patent Publications 20100092442, 20110318386, 20120003197 and U.S. Pat. Nos. 8,524,222, 8,246,965 and 8,025,875, all to Jacobsen et al. disclose the use of non-pathogenic bacterial isolates for induction of systemic acquired resistance (SAR) pathogenic infection, including salicylic acid accumulation, induction of defense proteins and release of ROS.

Some proposed methods employ transgenic and recombinant technology for increasing resistance or tolerance to the pathogen. US Patent Publication 20130205443 to Mirkov et al. discloses methods for enhancing pathogen resistance in citrus trees by administering to or transgenically expressing in the trees one or more anti-microbial peptides, such as plant defensins, chitinases, and the like.

US Patent Publication 20100122376 to Zipfel et al., discloses methods for enhancing citrus tree's resistance to plant bacterial pathogens by transgenically expressing, in the tree, an EF-Tu receptor protein, and intensifying the host tree's response (PAMP-triggered immunity and effector-triggered immunity) to the pathogen's EF-Tu elongation factor.

US Patent Publication 20090036307 to Gabriel et al., discloses methods for interfering with a bacterial infection of citrus trees by application, or expression of the potentially protective bacteriophage Bacterial Outer Membrane Breaching protein (BOMBp).

US Patent Publication 20130318652 to Messier discloses the transgenic expression of a plant defense protein, the dirigent protein, for conferring enhanced resistance to HLB infection and disease symptoms. US Patent publication 20080163390 to Kachroo et al. discloses inhibition of fatty acid desaturases for enhancing plant pathogen resistance response, through enhanced signaling intermediates.

However, to date, overexpression of plant pathogen resistance (PR) proteins in plants has not resulted in enhanced resistance (Conrath et al, 2006), and the complexity of HLB infection and disease has confounded efforts to boost citrus tree's resistance to the HLB epidemic through direct application or genetic modification.

### SUMMARY OF THE INVENTION

According to an aspect of some embodiments of the present invention there is provided a method of increasing yield, growth rate, vigor, biomass, fruit quality or stress tolerance of a citrus plant when infected with a plant pathogen, the method comprising introducing into the citrus plant an isolated nucleic acid agent comprising a nucleic acid sequence which specifically reduces the expression of at least one plant pathogen resistance gene product of the plant, thereby modulating at least one plant pathogen resistance response and increasing yield, growth rate, vigor, biomass, fruit quality or stress tolerance of the citrus plant when infected with a plant pathogen.

According to an aspect of some embodiments of the present invention there is provided a method of increasing yield, growth rate, vigor, biomass, fruit quality or stress tolerance of a plant when infected with a Candidatus Liberibacter spp, the method comprising introducing into the plant an isolated nucleic acid agent comprising a nucleic acid sequence which specifically reduces the expression of at least one plant pathogen resistance gene product of the plant, thereby modulating at least one plant pathogen resistance response and increasing yield, growth rate, vigor, biomass, fruit quality or stress tolerance of the plant when infected with a Candidatus Liberibacter Spp.

According to some embodiments of the present invention the plant pathogen is a Candidatus Liberibacter spp.

According to some embodiments of the present invention the plant is a citrus plant or a Solanaceous plant.

According to some embodiments of the present invention the plant is a citrus plant.

According to some embodiments of the present invention the method further comprises monitoring symptoms of infection in the infected plant following introducing.

According to some embodiments of the present invention the Candidatus Liberibacter spp is selected from the group consisting of Candidatus Liberibacter asiaticus, Candidatus Liberibacter africanus, Candidatus Liberibacter americanus and Candidatus Liberibacter psyllaurous.

According to some embodiments of the present invention the pathogen is Candidatus Liberibacter asiaticus (CaLas).

According to some embodiments of the present invention the plant, when infected, is suffering from HuangLongBing disease (HLB or citrus greening).

According to some embodiments of the present invention the plant pathogen resistance response is selected from the group consisting of changes in Salicylic acid levels, changes in Jasmonic acid levels, changes in Gibberellic acid levels, changes in Auxin levels, changes in Cytokinin levels, changes in Ethylene levels, changes in ABA phyto-hormones levels, up-regulation of phyto-hormone-related genes, biosynthesis, deposition and degradation of callose, reactive oxygen species production, functional FLS2/BAK1 complex formation, protein-kinase pathway activation, phloem blockage, starch accumulation, starch accumulation in phloem parenchyma cells, polymer sieve formation, changes in carbohydrate metabolism, cambial activity aberrations, sucrose accumulation and carotenoid synthesis.

According to some embodiments of the present invention the increase in yield, growth rate, vigor, biomass, fruit quality or stress tolerance is a change in a parameter selected from the group consisting of increased water uptake, increased plant height, increased plant flower number, decreased starch accumulation and decreased Disease Sign Index.

According to some embodiments of the present invention the change in said parameter is measured at a time point selected from the group consisting of 2-3 weeks post infection, 3-4 weeks post infection, 5-7 weeks post infection, 1-2 months post infection, 2-4 months post infection, 4-6 months post infection, 5-8 months post infection and 5-12 months post infection.

According to some embodiments of the present invention the pathogen resistance response is selected from the group consisting of reactive oxygen species production, callose biosynthesis and deposition, phloem blockage and changes in carbohydrate metabolism.

According to some embodiments of the present invention the plant pathogen resistance gene product is selected from plant gene products having upregulated expression following infection of the plant with said plant pathogen.

According to some embodiments of the present invention the plant pathogen resistance gene product is selected from the group consisting of the polynucleotide sequences of Table IV.

According to some embodiments of the present invention the plant pathogen resistance gene product is selected from the group consisting of plant gene products having at least 60% identity to any of the polynucleotide sequences of Table II.

According to some embodiments of the present invention the plant pathogen resistance gene product is selected from the group consisting of the polynucleotide sequences of Table V.

According to some embodiments of the present invention the plant pathogen resistance gene product is selected from SEQ ID NOs: 204-265 and 489-516 or homologs thereof.

According to some embodiments of the present invention, the plant pathogen resistance gene product is selected from SEQ ID NOs. 1-203 and homologs thereof.

According to some embodiments of the present invention the plant pathogen resistance gene product is selected from the group consisting of plant gene products having at least 60% identity to any of the polynucleotide sequences of Table III.

According to some embodiments of the present invention the plant pathogen resistance gene product is selected from the group consisting of an ADP-glucose pyrophosphorylase large subunit (AGPase) gene product, a glucose-6-phosphate/phosphate translocator (GPT) gene product, a Callose synthase gene product and a Myb transcriptional regulator (MYB) gene product.

According to some embodiments of the present invention, the nucleic acid sequence comprises a nucleic acid sequence selected from the group consisting of SEQ ID NOs. 528, 530, 532 and 536.

According to some embodiments of the present invention the plant pathogen resistance gene is selected from SEQ ID NOs: 623-714 or homologs thereof.

According to some embodiments of the present invention the introducing is affected via spraying, dusting, soaking, injecting, aerosol application, particle bombardment, irrigation or via positive or negative pressure application.

According to some embodiments of the present invention the plant is a fruit tree.

According to some embodiments of the present invention the fruit tree is a citrus tree.

According to some embodiments of the present invention the isolated nucleic acid agent further comprises a cell penetrating agent.

According to some embodiments of the present invention, introducing is following detection of a symptom of infection of the plant with the pathogen.

According to an aspect of some embodiments of the present invention there is provided an isolated nucleic acid agent comprising a nucleic acid sequence which specifically reduces the expression of at least one plant pathogen resistance gene product selected from the group consisting of plant gene products having at least 60% identity to any of the polynucleotide sequences of Table II.

According to some embodiments of the present invention the isolated nucleic acid agent is a dsRNA.

According to some embodiments of the present invention the dsRNA is selected from the group consisting of siRNA, shRNA and miRNA.

According to some embodiments of the present invention the nucleic acid sequence is greater than 15 base pairs in length.

According to some embodiments of the present invention the nucleic acid sequence is 19 to 25 base pairs in length.

According to some embodiments of the present invention the nucleic acid sequence is 30-100 base pairs in length.

According to some embodiments of the present invention the nucleic acid sequence is 100-500 base pairs in length.

According to some embodiments of the present invention the plant pathogen resistance gene product is selected from plant gene products having upregulated expression following infection of the plant with said plant pathogen.

According to some embodiments of the present invention the plant pathogen resistance gene is a HuangLongBing-associated plant pathogen resistance gene.

According to some embodiments of the present invention the isolated nucleic acid agent comprises a nucleic acid sequence selected from the group consisting of the polynucleotide sequences of Table IV and IV(a).

According to some embodiments of the present invention there is provided a nucleic acid construct comprising a nucleic acid sequence encoding the isolated nucleic acid agent of the invention.

According to some embodiments of the present invention the nucleic acid construct further comprises a regulatory element active in plant cells.

According to some embodiments of the present invention the nucleic acid construct comprises a viral silencing vector comprising a viral genome or portion thereof.

According to some embodiments of the present invention the nucleic acid construct the viral genome or portion thereof is sufficient to effect viral induced gene silencing.

According to some aspects of some embodiments of the present invention there is provided a bacterial host cell comprising the nucleic acid construct of the invention.

According to some embodiments of the present invention the bacterial cell is an Agrobacterium.

According to an aspect of some embodiments of the present invention there is provided a citrus plant comprising at least one exogenous isolated nucleic acid agent comprising a nucleic acid sequence which specifically reduces the expression of at least one plant pathogen resistance gene product.

According to an aspect of some embodiments of the present invention there is provided a plant comprising at least one exogenous isolated nucleic acid agent comprising a nucleic acid sequence which specifically reduces the expression of at least one plant pathogen resistance gene product selected from the group consisting of plant gene products having at least 60% identity to any of the polynucleotide sequences of Table II.

According to some embodiments of the present invention the plant is selected from the group consisting of a tree, a shrub, a bush, a seedling, a scion, a rootstock, an inarched plant, a bud, a budwood, a root and a graft.

According to some embodiments of the present invention the plant is a citrus or citrus-related plant.

According to some embodiments of the present invention the plant is a plant at risk of infection with CaLas.

According to some embodiments of the present invention there is provided a cell of the plant of the invention.

According to an aspect of some embodiments of the present invention there is provided an agrochemical composition comprising an isolated nucleic acid agent comprising a nucleic acid sequence which specifically reduces the expression of at least one plant pathogen resistance gene product in a plant and a plant-beneficial compound selected from the group consisting of a fertilizer, an antibiotic, a biocide, a pesticide, a pest repellent, an herbicide, a plant hormone.

According to some embodiments of the present invention the agrochemical composition comprises the isolated nucleic acid agent of the invention or the nucleic acid construct of the invention.

According to some embodiments of the present invention the agrochemical composition, isolated nucleic acid agent or the nucleic acid construct of the invention is formulated in a formulation selected from the group consisting of an aerosol, a dust, a dry flowable, an emulsifiable flowable, a granule, a microencapsulation, a pellet, a soluble powder, a wettable powder, a liquid and a water dispersible granule.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows the effect of PDS (phytoene desaturase) gene silencing using Agrobacterium transformation of TRV VIGS on general phenotype of three tomato cultivars, at 19 and 24 days post infection (dpi). EV—empty vector. Note the progressive photo-bleaching of the leaves in the selected cultivars;

FIG. 2 is a graph showing detection of PDS gene expression in tomato leaves, indicating the correlation between the silencing of PDS and the leaf color phenotype (green v white). TRV EV is empty vector control;

FIG. 3 is an illustration depicting the etiology, along an 80 day time course, of disease in tomatoes infected with C. Liberibacter solanacearum (Lso);

FIG. 4 is a photograph showing the difference in height between infected Tiny Tom tomato plants (right) and uninfected controls (left);

FIG. 5 is a photograph showing the difference in flower number between infected Tiny Tom tomato plants (right) and uninfected controls (left);

FIG. 6 is a photograph of an agarose gel showing the results of PCR detection of Lso 16S DNA, corresponding to the expected size in psyllid-rearing infected (19, 20, 23 and 24) but not non-infected (N1, N2) plants;

FIG. 7 is a photograph of an agarose gel showing the results of PCR detection of Lso 16S, using cDNA, corresponding to the disease phenotype (intense bands correspond to severity of the disease symptoms in plants);

FIG. 8 is a photograph of an agarose gel verifying the identity and integrity of the inserts of the Agrobacterium clones harboring sequences for gene silencing of selected targets, as well as the empty pTRV1 and control (MCS-multiple cloning site-TRV2 w/o additional sequences) clones;

FIG. 9 is a graph showing the Ct (cycle threshold) and silencing ratio for the different target genes, indicating that the silencing ratio (fold decrease of transcript relative to EV control) is inversely proportional to basal expression level (as expressed in Ct);

FIG. 10 is table showing the small RNA abundance and distribution in plants which were exposed to silencing via VIGS;

FIG. 11 are photographs depicting the phenotypic parameters which make up the Disease Severity Index: DSI 0—normal, healthy plant; 1—slight stunting, suggestion of curling; 2—some stunting, clear curling, stiffness and springiness of leaves; 3—notable stunting, curled and thickened leaves, midrib stiffness and springiness, some purpling; and 4—severe stunting, extremely stiff and thickened, purplish and dying leaves, complete lack of growth and fruit;

FIG. 12 depicts the grafting procedure for infecting citrus trees/plants with HLB (C. Liberibacter spp);

FIG. 13 is a photograph of an agarose gel of PCR products verifying the presence of C. Liberibacter 16S DNA (see “+” positive control) in HLB-infected trees (lanes 73, 101, 105, 112 and 171);

FIG. 14 is a graph showing the up-regulation of expression of the PP2 gene in citrus, in response to HLB infection (normalized expression is calculated using delta Ct against the citrus 18S gene normalizing transcript);

FIG. 15 is a graph showing the up-regulation of expression of the AGPase gene in citrus, in response to HLB infection (normalized expression is calculated using delta Ct against the citrus 18S gene normalizing transcript);

FIG. 16 is a graph showing the up-regulation of expression of the GPT gene in citrus, in response to HLB infection (normalized expression is calculated using delta Ct against the citrus 18S gene normalizing transcript);

FIG. 17 is a graph showing the up-regulation of expression of the alpha-amylase gene in citrus, in response to HLB infection (normalized expression is calculated using delta Ct against the citrus 18S gene normalizing transcript);

FIG. 18 is a graph showing the up-regulation of expression of the oxidoreductase gene in citrus, in response to HLB infection (normalized expression is calculated using delta Ct against the citrus 18S gene normalizing transcript);

FIG. 19 is a graph showing the up-regulation of expression of the CSD1 gene in citrus, in response to HLB infection (normalized expression is calculated using delta Ct against the citrus 18S gene normalizing transcript);

FIG. 20 is a graph showing the upregulation of MYB gene expression in HLB infected citrus trees (red columns, HLB positive), as assayed by signal amplification, at one, three and 6 months post-grafting (infection);

FIG. 21 is a graph showing the upregulation of zinc transporter 5 gene expression in HLB infected citrus trees (red columns, HLB positive), as assayed by signal amplification, at one, four and 6 months post-grafting (infection);

FIG. 22 is a graph showing the upregulation of PP2 gene expression in HLB infected citrus trees (red columns, HLB positive), as assayed by signal amplification, at one, four and 6 months post-grafting (infection);

FIG. 23 is a graph showing the upregulation of superoxide dismutase gene expression in HLB infected citrus trees (red columns, HLB positive), as assayed by signal amplification, at one, four and 6 months post-grafting (infection);

FIG. 24 is a graph showing the upregulation of AGPase gene expression in HLB infected citrus trees (red columns, HLB positive), as assayed by signal amplification, at one, three and 6 months post-grafting (infection);

FIG. 25 is a graph illustrating the increased starch accumulation in leaves of HLB infected citrus trees (red columns, HLB positive) at 6 months post-grafting (infection);

FIG. 26 is a graph illustrating the altered dynamics of starch accumulation in leaves of HLB infected citrus trees (red columns, HLB positive), measured at 08:00, 14:00 and 20:00, standardized to the starch content of healthy leaves at 8:00;

FIG. 27 shows statistical analysis of the linear best fit of the results of starch accumulation dynamics shown in FIG. 26;

FIG. 28 is a graph showing the effects of GPT silencing by VIGS agro-infusion on GPT expression in tomato plants, measured as relative expression two weeks post-Lso infection (red columns are infected plants);

FIG. 29 is a graph showing the effects of lipoxygenase D (LoxD) silencing by VIGS agro-infusion on LoxD expression in tomato plants, measured as relative expression two weeks post-Lso infection (red columns are infected plants);

FIG. 30 is a graph showing the effects of Myb transcriptional regulator (MYB) silencing by VIGS agro-infusion on MYB expression in tomato plants, measured as relative expression two weeks post-Lso infection (red columns are infected plants);

FIG. 31 is a graph showing the effects of AGPase silencing by VIGS agro-infusion on AGPase expression in tomato plants, measured as relative expression two weeks post-Lso infection (red columns are infected plants);

FIG. 32 is a series of graphs showing the effects of gene silencing (by VIGS agro-infusion) of candidate genes Myb, LoxD, CalS, PP2, AGPase and GPT on the phenotype (DSI) of infected tomato plants 2 and 3 weeks post-Lso infection (red columns are infected plants), compared to empty vector (EV) and untreated control plants;

FIG. 33 is a series of graphs showing the effects of gene silencing (by VIGS agro-infusion) of candidate genes Myb, CalS, PP2, AGPase and GPT on the phenotype (flower number) of infected tomato plants 2 and 3 weeks post-Lso infection (red columns are infected plants), compared to empty vector (EV) and untreated control plants;

FIG. 34 is a graph showing the effects of gene silencing (by VIGS agro-infusion) of candidate genes Myb, CalS, PP2, AGPase and GPT on the phenotype (water uptake) of infected tomato plants 5 weeks post-Lso infection (red columns are infected plants), compared to empty vector (EV) and untreated control plants.

### DESCRIPTION OF SPECIFIC EMBODIMENTS OF THE INVENTION

The present invention, in some embodiments thereof, relates to methods for enhancing fitness of pathogen-infected plants, and, more particularly, but not exclusively, to methods of using RNA interference for modulation of plant-pathogen resistance response gene expression. The present invention discloses compositions for silencing of specific plant pathogen resistance response genes with siRNA in pathogen-susceptible plants, reducing the negative impact of the plant pathogen resistance response upon infection of the host plant and enhancing fitness. In particular, the present invention provides compositions and methods for enhancing host plant fitness and fruit yield and quality following Candidatus Liberibacter spp infection and, specifically, Candidatus Liberibacter spp infection in citrus plants and trees, as in Huang Long Bing.

Before explaining at least one embodiment of the disclosure in detail, it is to be understood that the disclosure is not necessarily limited in its application to the details set forth in the following description or exemplified by the Examples. The disclosure is capable of other embodiments or of being practiced or carried out in various ways. Also, it is to be understood that the phraseology and terminology employed herein is for the purpose of description and should not be regarded as limiting.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 527 is expressed in a DNA sequence format (e.g., reciting T for thymine), but it can refer to either a DNA sequence that corresponds to an alpha-amylase nucleic acid sequence, or the RNA sequence of an RNA molecule (e.g. reciting U for uridine) that corresponds to the RNA sequence shown. In any event, both DNA and RNA molecules having the sequences disclosed with any substitutes are envisioned.

Plant immune response provides protection against a variety of phytopathogenic organisms, including bacteria, fungi, nematodes, viruses, mollicutes (mycoplasmas, spiroplasmas), protozoa, phanerogams; rickettsias, and viroids, insects and parasitic plants. The two-tiered system of plant innate immune response to pathogen insult or invasion [microbial/pathogen-associated molecular pattern- (PAMP or MAMP) triggered immunity, or PTI, and effector-triggered immunity, or ETI) can be divided into characteristic stages: In stage 1, plants detect MAMPs and/or PAMPs via transmembrane pattern recognition receptors (PRRs, such as Receptor-Like Kinases, RLKs), triggering PTI. The plant's initial response to the pathogen insult is mediated by numerous, somewhat overlapping signaling cascades (salicylic acid signaling is critical) and includes molecular, morphological and physiological changes. Early changes occurring within seconds to minutes include ion-flux across the plasma membrane, phytoalexin synthesis, an oxidative burst, mitogen activated protein (MAP) kinase activation and protein phosphorylation, followed by substantial transcriptional reprogramming within the first hour of PTI. Later changes include pathogenesis-related protein synthesis, callose deposition, which serves as a physical barrier at infection sites, and stomatal closure.

In stage 2, virulent pathogens respond to the PRR-based defenses by deploying effectors into the host cell to evade or suppress PTI responses. These in turn activate stage 3, in which ETI, mediated by intracellular nucleotide-binding domain Leucine-Rich Repeat (LRR) proteins, leads to growth inhibition and often accompanying hypersensitive response.

The hypersensitive response involves a form of programmed cell death at the site of the pathogen invasion. The hypersensitive response is characterized by cytoplasmic shrinkage, chromatin condensation, mitochondrial swelling, vacuolization and chloroplast disruption. Molecular events underlying the hypersensitive response include down-regulation of photosynthesis, increased production and accumulation of reactive oxygen species, reactive nitrogen oxide intermediates and the defense hormones salicylic and jasmonic acid, activation of MAPK cascades changes in intracellular calcium levels and transcriptional reprogramming, however, with greater amplitude and acceleration than in the PTI stage.

Pathogen infection also produces a type of systemic response in the plant, remote from the site of infection, “priming” other plant organs and tissues for pathogen insult. The systemic acquired response (SAR) prepares the unaffected tissues for contact with the pathogens by mobilizing pathogenesis related proteins, signaling and synthetic pathways, allowing for swift and heightened response in the event of pathogen insult in unaffected tissues, while economically avoiding the initiation of a full-blown response until actually challenged. The systemic signals mediating SAR have not been fully elucidated, but salicylic acid has been confirmed as an important signaling intermediate.

The result of the PTI, ETI, hypersensitive response and SAR, in response to a pathogen insult, is often significant re-allocation of energy resources and growth inhibition, isolation of the affected region and, ultimately cell death and necrosis of affected regions. P-protein accumulation and callose formation act to occlude sieve elements in the vascular system of pathogen-infected tissue, blocking pathogen and pathogen effector dispersal, resulting in bidirectional disruption of water, metabolite and hormone transport.

Thus, while the plant pathogen responses act to effectively isolate the affected tissues and limit pathogen reproduction, plant pathogen defenses involve significant energy expenditure, metabolic and morphological re-organization, ultimately detrimental to plant vigor, fitness, growth and crop (i.e. fruit, seed, etc) production. Of particular importance is the common re-infection by plant pathogens, for example by repeated contact with pathogen-bearing insect vectors effective in disseminating the pathogenic organisms, and the recurrent activation of the plant pathogen response, depletion of plant resources and subsequent loss of host plant vigor.

RNA interference (dsRNA and siRNA) strategies have been shown to be effective in silencing gene expression in a broad variety of species, including plants.

RNA interference (RNAi) inhibits gene expression in a sequence specific fashion, occurring in at least two steps: The first step cleaves a longer dsRNA into shorter, 21- to 25-nucleotide-long dsRNAs, termed “small interfering RNAs” or siRNAs. In the second step, the smaller siRNAs then mediate the degradation of a target corresponding mRNA molecule. This RNAi effect can be achieved by introduction of either longer double-stranded RNA (dsRNA) or shorter small interfering RNA (siRNA) to the target sequence within cells.

RNAi has been successfully demonstrated in plant-pest management. Plants possess an innate RNA interference capability, similar but not identical to animal RNAi, highly effective in preventing spread of viral pathogens. Also, as most insects are susceptible to RNAi gene silencing by dsRNA, expression of pest-specific dsRNA in transgenic plants, as well as direct application of dsRNA to the insect pests can afford protection from plant-pest injury and damage. The introduction of dsRNA into transgenic plants can be highly specific to target pathogens. Indeed, gene silencing by dsRNA has been demonstrated effective for plant-pest control and plant-virus control, for example, US Patent Publication No. 20110150839 to Arciello et al discloses the transgenic expression, in a plant host, of a construct encoding a pathogen GPCR receptor-specific dsRNA for enhancing the resistance of the plant to attack by phytopathogenic organisms.

Resistance to Potato-Y virus, Cucumber and Tobacco Mosaic Virus, Tomato Spotted Wilt Virus, Bean Golden Mosaic Virus, Banana Bract Mosaic Virus, and Rice Tungro Bacilliform Virus among many others has been demonstrated in transgenic plants expressing viral-specific RNA transcripts. Transgenic expression of fungal-specific dsRNA effectively conferred resistance to barley powdery mildew Blumeria graminis in barley.

It has been found that direct application of dsRNA is also effective in plants—for example, viral-specific double-stranded RNA (dsRNA) from the tobamovirus, potyvirus, and alfamovirus groups, when directly delivered to leaf cells, effectively inhibited infection of the plants. Methods for introduction of dsRNA into seeds, shoots, plant cell culture and all tissues and organs of adult plants are well known in the art.

The present inventors propose to reduce the deleterious effects of the plant pathogen resistance response on plant fitness, growth, yield and fruit quality by modulating plant-pathogen resistance response gene expression using RNAi gene silencing of endogenous pathogen resistance response genes, in citrus or other Candidatus Liberibacter spp-susceptible host plants. The present inventors have identified target pathogen-resistance response-associated genes and designed nucleic acid agents for RNAi silencing which, when provided to the plant, improve the fitness, vigor, yield, fruit quality and other traits of the plant following pathogen infection.

Thus, according to some embodiments of aspects of the invention there is provided a method of increasing yield, growth rate, vigor, biomass, stress tolerance or fruit quality of a citrus plant when infected with a plant pathogen, the method comprising introducing into the citrus plant an isolated nucleic acid agent comprising a nucleic acid sequence which specifically reduces the expression of at least one plant pathogen resistance gene product of a plant, thereby modulating at least one plant pathogen resistance response and increasing yield, growth rate, vigor, biomass, fruit quality or stress tolerance of the citrus plant when infected with the plant pathogen.

In yet another embodiment of the present invention, there is provided a method of increasing yield, growth rate, vigor, biomass, stress tolerance or fruit quality of a plant when infected with a Candidatus Liberibacter spp bacteria, the method comprising introducing into the plant an isolated nucleic acid agent comprising a nucleic acid sequence which specifically reduces the expression of at least one plant pathogen resistance gene product of the plant, thereby modulating at least one plant pathogen resistance response and increasing yield, growth rate, vigor, biomass, fruit quality or stress tolerance of the plant when infected with the Candidatus Liberibacter spp bacteria.

The term “plant” as used herein encompasses whole plants, ancestors and progeny of the plants and plant parts, including seeds, shoots, stems, roots (including tubers), and isolated plant cells, tissues and organs. The plant may be in any form including suspension cultures, embryos, meristematic regions, callus tissue, leaves, gametophytes, sporophytes, pollen, and microspores. It will be appreciated, that the plant or seed thereof may be transgenic plants.

As used herein the phrase “plant cell” refers to plant cells which are derived and isolated from disintegrated plant cell tissue or plant cell cultures. The phrase “plant cell” may also refer to plant cells “in situ”, e.g. cells of plant tissue, which are not isolated from the tissue or plant organ.

As used herein the phrase “plant cell culture” refers to any type of native (naturally occurring) plant cells, plant cell lines and genetically modified plant cells, which are not assembled to form a complete plant, such that at least one biological structure of a plant is not present. Optionally, the plant cell culture of this embodiment of the present disclosure may comprise a particular type of a plant cell or a plurality of different types of plant cells. It should be noted that optionally plant cultures featuring a particular type of plant cell may be originally derived from a plurality of different types of such plant cells. In certain embodiments according to the present disclosure, the plant cell is a non-sexually producing plant cell. In other aspects, a plant cell of the present disclosure is a non-photosynthetic plant cell.

Any commercially or scientifically valuable plant susceptible to infection by Candidatus Liberibacter spp. is envisaged in accordance with some embodiments of the invention. Plants that are particularly useful in the methods of the disclosure include, but are not limited to:

Plants from the Rutaceae family such as all citrus species and subspecies, including sweet oranges commercial varieties (Citrus sinensis Osbeck (L.), clementines (C. reticulata), limes (C. aurantifolia), lemon (C. limon), sour orange (C. aurantium), hybrids and relatives (Citranges, Citrumelos, Citrandarins), Balsamocitrus dawei, C. maxima, C. jambhiri, Clausena indica, C. lansium, Triphasia trifolia, Swinglea glutinosa, Micromellum tephrocarpa, Merope spp., Eremolemon; Atalantia spp., Severinia buxifolia; Microcitrus spp., Fortunella spp., Calodendrum capense, Murraya spp., Poncirus trifoliate;

Plants of the Solanaceae family such as Tobacco (Nicotiana spp.), Tomato (Lycopersicon esculentum), Potato (Solanum tuberosum), Capsicum (Capsicum annuum), Cape gooseberry (Physalis peruviana), Tomato tree or Tamarillo (Solanum betaceum);

Plants from Rosaceae family such as Pear (Pyrus communis);

Plants from Apiaceae family such as Carrot (Daucus carota);

Plants from Convolvulaceae family such as Dodder (Cuscuta spp.)

Plants from Apocynaceae family such as: Vinca (Catharanthus roseus).

According to some embodiments, the plant used by the method of the invention is a crop plant.

According to a specific embodiment, the plant is selected from the group consisting of citrus plants, including, but not limited to all citrus species and subspecies, including sweet oranges commercial varieties (Citrus sinensis Osbeck (L.), clementines (C. reticulata), limes (C. aurantifolia), lemon (C. limon), sour orange (C. aurantium), hybrids and relatives (Citranges, Citrumelos, Citrandarins), Balsamocitrus dawei, C. maxima, C. jambhiri, Clausena indica, C. lansium, Triphasia trifolia, Swinglea glutinosa, Micromellum tephrocarpa, Merope spp., Eremolemon; Atalantia spp., Severinia buxifolia; Microcitrus spp., Fortunella spp., Calodendrum capense, Murraya spp. and Poncirus trifoliate. In some embodiments the citrus plant is an orange, a lemon, a lime, a grapefruit, a clementine, a tangerine or a pornello tree. The citrus tree can be a seed-grown tree or a grafted tree, grafted onto a different citrus rootstock.

As used herein, the phrase “plant pathogen” or “phytopathogen” refers to a nucleic acid-containing agent capable of proliferation within the plant cell or plant, the pathogen causing disease in the plant, by disrupting normal function and/or growth of the plant, usually by invasion of the plant cell, and exploiting the plant cell nutrients, metabolites and/or energy metabolism for pathogen reproduction. Plant pathogenic organisms include pathogenic viruses, bacteria, fungi, oomycetes, Ascomycetes, Basidomycetes, nematodes, mollicutes (mycoplasmas, spiroplasmas), protozoa, phanerogams, rickettsias, and viroids, insects and parasitic plants.

A plant pathogen can be an intracellular or extra-cellular pathogen. Table I below includes a non-exhaustive list of exemplary plant pathogens which cause or facilitate the indicated disease in the indicated host plant, the response to which is amenable to modulation by the compositions and methods of the present invention.

TABLE I

| Host plant | Disease | Bacterial pathogen |
|---|---|---|
| Tomato | Bacterial canker | Clavibacter michiganensis subsp. michiganensis |
| Tomato | Bacterial speck | Pseudomonas syringae pv. tomato |
| Tomato | Bacterial spot | Xanthomonas campestris pv. vesicatoria |
| Tomato | Bacterial stem rot and fruit rot | Erwinia carotovora subsp. carotovora |
| Tomato | Bacterial wilt | Ralstonia solanacearum |
| Tomato | Pith necrosis | Pseudomonas corrugata |
| Tomato | Syringae leaf spot | Pseudomonas syringae pv. syringae |
| Tomato | Psyllid yellowing | Candidatus Liberibacter psyllaurous |
| Tomato | Zebra chip | Candidatus Liberibacter solanacearum (Lso) |
| Citrus | Bacterial spot | Xanthomonas campestris pv. citrumelo |
| Citrus | Black pit (fruit) | Pseudomonas syringae |
| Citrus | Blast | Pseudomonas syringae |
| Citrus | Citrus canker | Xanthomonas axonopodis = |
| | | Xanthomonas campestris pv. citri |
| Citrus | Citrus stubborn | Spiroplasma citri |
| Citrus | Citrus variegated chlorosis | Xylella fastidiosa |
| Citrus | Huanglongbing = citrus | Candidatus Liberibacter asiaticus; |
| | greening | Candidatus Liberibacter africanus; |
| | | Candidatus Liberibacter americanus |

According to one embodiment of the invention, the pathogen is a bacteria, causing or facilitating Huanglongbing disease, such as Candidatus Liberibacter asiaticus, Candidatus Liberibacter africanus, Candidatus Liberibacter americanus and the like. According to another embodiment of the invention, the pathogen is a bacteria, causing or facilitating a disease in tomato such as zebra chip disease (Candidatus Liberibacter solanacearum (Lso)), psyllid yellowing (Candidatus Liberibacter psyllaurous) and the like.

As used herein, the terms “plant disease” or “pathogen infection” is defined as undesirable changes in the physiology, morphology, reproductive fitness, economic value, vigor, biomass, fruit quality, stress-tolerance, resistance to infection and/or infestation of a plant, directly or indirectly resulting from contact with a plant pathogenic agent. According to one embodiment of the invention, the undesirable changes include, but are not limited to biomass and/or yield of the diseased or pathogen infected plant. According to another embodiment of the invention, change in yield includes, but is not limited to change in fruit yield, fruit quality, seed yield, flower yield, crop yield and the like. In some embodiments, the host plant is a citrus tree or bush and the plant disease or pathogenic infection is a Candidatus Liberibacter infection, in particular, a Candidatus Liberibacter asiaticus infection. In some embodiments, the Candidatus Liberibacter infection causes HLB disease in the host plant.

HLB disease, caused by infection of a susceptible host plant (e.g. a citrus plant) with a Candidatus Liberibacter pathogen, such as, but not limited to C. Liberibacter asiaticus, is characterized by asymmetric blotchy mottling of older leaves, chlorotic patterns, twig-dieback, reduced fruit production, premature fruit drop and eventually tree decline, most likely due to blockage of the translocation stream by plugging of sieve elements along with phloem necrosis. Thus, in some embodiments of some aspects of the invention, introducing the isolated nucleic acid agent of the invention into the susceptible plant (e.g. citrus tree) results in reduced mottling and fewer chlorotic patterns in the leaves, reduced twig die-back, improved fruit production, prevention of premature fruit drop, increased vigor and delay or prevention of decline in treated plants and trees following Candidatus Liberibacter infection, as compared to identical untreated plants and trees, following infection by Candidatus Liberibacter spp.

According to some embodiments of the invention, reducing expression of at least one plant pathogen resistance response gene product in a plant increases at least one of yield, growth rate, vigor, biomass or stress tolerance of the plant following pathogen infection, compared to a similar or identical plant having normal expression of the at least one plant pathogen resistance response gene product, following pathogen infection.

As used herein, the phrase “stress tolerance” refers to both tolerance to biotic stress, and tolerance to abiotic stress. The phrase “abiotic stress” as used herein refers to any adverse effect on metabolism, growth, viability and/or reproduction of a plant caused by a-biotic agents. Abiotic stress can be induced by any of suboptimal environmental growth conditions such as, for example, water deficit or drought, flooding, freezing, low or high temperature, strong winds, heavy metal toxicity, anaerobiosis, high or low nutrient levels (e.g. nutrient deficiency), high or low salt levels (e.g. salinity), atmospheric pollution, high or low light intensities (e.g. insufficient light) or UV irradiation. Abiotic stress may be a short term effect (e.g. acute effect, e.g. lasting for about a week) or alternatively may be persistent (e.g. chronic effect, e.g. lasting for example 10 days or more). The present disclosure contemplates situations in which there is a single abiotic stress condition or alternatively situations in which two or more abiotic stresses occur.

As used herein the phrase “abiotic stress tolerance” refers to the ability of a plant to endure an abiotic stress without exhibiting substantial physiological or physical damage (e.g. alteration in metabolism, growth, viability and/or reproducibility of the plant).

According to some embodiments, reducing expression of at least one plant pathogen resistance response gene product in a pathogen-infected plant increases crop production. Crop production can be measured by biomass, vigor or yield, and can be used to calculate nitrogen use efficiency and fertilizer use efficiency. As used herein, the phrase “nitrogen use efficiency (NUE)” refers to a measure of crop production per unit of nitrogen fertilizer input. Fertilizer use efficiency (FUE) is a measure of NUE. The plant's nitrogen use efficiency is typically a result of an alteration in at least one of the uptake, spread, absorbance, accumulation, relocation (within the plant) and use of nitrogen absorbed by the plant. Improved crop production, vigor, yield, NUE or FUE is with respect to that of a pathogen-infected or diseased plant not having reduced expression of the at least one plant pathogen resistance gene product (i.e., lacking the nucleic acid agent of the invention) of the same or similar species and developmental stage and grown under the same or similar conditions.

As used herein the term/phrase “biomass”, “biomass of a plant” or “plant biomass” refers to the amount (e.g., measured in grams of air-dry tissue) of a tissue produced from the plant in a growing season. An increase in plant biomass can be in the whole plant or in parts thereof such as aboveground (e.g. harvestable) parts, vegetative biomass, roots and/or seeds or contents thereof (e.g., oil, starch etc.).

As used herein the term/phrase “vigor”, “vigor of a plant” or “plant vigor” refers to the amount (e.g., measured by weight) of tissue produced by the plant in a given time. Increased vigor could determine or affect the plant yield or the yield per growing time or growing area. In addition, early vigor (e.g. seed and/or seedling) results in improved field stand.

As used herein the term/phrase “yield”, “yield of a plant” or “plant yield” refers to the amount (e.g., as determined by weight or size) or quantity (e.g., numbers) of tissues or organs produced per plant or per growing season. Increased yield of a plant can affect the economic benefit one can obtain from the plant in a certain growing area and/or growing time.

According to one embodiment the yield is measured by cellulose content, oil content, starch content and the like.

According to another embodiment the yield is measured by oil content.

According to another embodiment the yield is measured by protein content.

According to another embodiment, the yield is measured by seed number, seed weight, flower number or flower weight, fruit number or fruit weight per plant or part thereof (e.g., kernel, bean).

A plant yield can be affected by various parameters including, but not limited to, plant biomass; plant vigor; plant growth rate; seed yield; seed or grain quantity; seed or grain quality; oil yield; content of oil, starch and/or protein in harvested organs (e.g., seeds or vegetative parts of the plant); flower development, number of flowers (e.g. florets) per panicle (e.g. expressed as a ratio of number of filled seeds over number of primary panicles); harvest index; number of plants grown per area; number and size of harvested organs per plant and per area; number of plants per growing area (e.g. density); number of harvested organs in field; total leaf area; carbon assimilation and carbon partitioning (e.g. the distribution/allocation of carbon within the plant); resistance to shade; resistance to lodging, number of harvestable organs (e.g. seeds, flowers), seeds per pod, weight per seed, flowers per plant; and modified architecture [such as increase stalk diameter, thickness or improvement of physical properties (e.g. elasticity)].

According to some embodiments of aspects of the invention, fruit quality and yield are increased by introduction into the plant of the nucleic acid agent. Fruit yield can be measured according to harvest index (see above), expressed as number and/or size of fruit per plant or per growing area, and/or according to the quality of the fruit-fruit quality can include, but is not limited to sugar content, appearance of the fruit, shelf life and/or suitability for transport of the fruit, ease of storage of the fruit, increase in commercial value, fruit weight, juice weight, juice weight/fruit weight, rind weight, TSS—total soluble solids (°Brix), seed quality, symmetry, dry weight, TA—titrable acidity, MI—maturity index, CI—Colour index, peel colour, nutraceutical properties vitamin C—ascorbic acid—content, hesperidin content, total flavonoids content and the like.

Improved plant NUE is translated in the field into either harvesting similar quantities of yield, while deploying less fertilizer, or increased yields gained by implementing the same levels of fertilizer. Thus, improved NUE or FUE has a direct effect on plant yield in the field.

As used herein “biotic stress” refers stress that occurs as a result of damage done to plants by other living organisms, such as bacteria, viruses, fungi, parasites, beneficial and harmful insects, weeds, and cultivated or native plants. It will be appreciated that, in some embodiments, improving or increasing vigor or growth rate of a plant pathogen infected or diseased plant according some aspects of some methods of the invention, while reducing the expression of at least one plant pathogen resistance response gene, contributes to the overall health and robustness of the plant, thereby conferring improved tolerance to biotic, as well as abiotic stress. Such biotic stress can be, for example, the result of infection with same pathogen(s) with which the infected or diseased plant was infected prior to introduction of the isolated nucleic acid agent, or with a different plant pathogen.

In some embodiments of the invention, introduction of the isolated nucleic acid agent of the invention into the plant, and modulation of the at least one plant pathogen resistance response results in: improved tolerance of abiotic stress (e.g., tolerance of water deficit or drought, heat, cold, non-optimal nutrient or salt levels, non-optimal light levels) or of biotic stress (e.g., crowding, allelopathy, or wounding); a modified primary metabolite (e.g., fatty acid, oil, amino acid, protein, sugar, or carbohydrate) composition; a modified secondary metabolite (e.g., alkaloids, terpenoids, polyketides, non-ribosomal peptides, and secondary metabolites of mixed biosynthetic origin) composition; a modified trace element (e.g., iron, zinc), carotenoid (e.g., beta-carotene, lycopene, lutein, zeaxanthin, or other carotenoids and xanthophylls), or vitamin (e.g., tocopherols) composition; improved yield (e.g., improved yield under non-stress conditions or improved yield under biotic or abiotic stress); improved ability to use nitrogen or other nutrients; modified agronomic characteristics (e.g., delayed ripening; delayed senescence; earlier or later maturity; improved shade tolerance; improved resistance to root or stalk lodging; improved resistance to “green snap” of stems; modified photoperiod response); modified growth or reproductive characteristics; improved harvest, storage, or processing quality (e.g., improved resistance to pests during storage, improved fruit harvest, fruit storage, or fruit processing quality (e.g., improved resistance to pests during storage, improved resistance to breakage, improved appeal to consumers); or any combination of these traits.

In some embodiments of the invention, introduction of the isolated nucleic acid agent of the invention into the plant, and modulation of the at least one plant pathogen resistance response results in changes in height, water uptake, number of flowers, starch accumulation and Disease Sign Index of the treated plants when infected with a Candidatus Liberibacter pathogen, relative to infected plants untreated with the nucleic acid agent. In some embodiments, the parameters are increased, such as number of flowers, height and water uptake, indicating improved phenotype of the treated plants in response to the infection with Candidatus Liberibacter pathogen. In other embodiments, parameters such as starch accumulation and disease sign index (DSI) are decreased in plants receiving the isolated nucleic acid agent of the invention into the plant, and undergoing modulation of the at least one plant pathogen resistance response, indicating improved phenotype of the treated plants in response to the infection with Candidatus Liberibacter pathogen.

As used herein the term “improving” or “increasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90% or greater increase in NUE, in tolerance to stress, in growth rate, in yield, in biomass, in fruit quality, in height, in flower number, in water uptake or in vigor of a plant, as compared to the same or similar plant infected with the same pathogen or having the same disease, and not having reduced expression of at least one plant pathogen resistance gene product (i.e., plant lacking the nucleic acid agent) of the disclosure.

As used herein the term “decreasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90% or greater decrease in disease signs such as DSI, starch accumulation and the like of a plant, as compared to the same or similar plant infected with the same pathogen or having the same disease, and not having reduced expression of at least one plant pathogen resistance gene product (i.e., plant lacking the nucleic acid agent) of the disclosure.

In some embodiments, the changes in height, water uptake, number of flowers, starch accumulation and Disease Sign Index of the treated plants when infected with a Candidatus Liberibacter pathogen, relative to infected plants untreated with the nucleic acid agent is measured at a time point 2-3 weeks post infection, 3-4 weeks post infection, 5-7 weeks post infection, 1-2 months post infection, 2-4 months post infection, 4-6 months post infection, 5-8 months post infection and 5-12 months post infection or more.

According to some embodiments of the invention, plant parameters are monitored in the treated plants following introduction of the nucleic acid agent. In some embodiments, parameters of plant pathogen resistance response are monitored, for example, expression of plant pathogen resistance response genes, and/or physiological or metabolic symptoms of the expression of such plant pathogen resistance response genes. In other embodiments, instead of, or in addition to monitoring parameters of plant pathogen resistance response gene expression, parameters of the plant's tolerance to stress, growth rate, yield, biomass, fruit quality or vigor of the plant can be monitored, and can be compared to similar parameters from plants lacking the nucleic acid agent of the invention. In some embodiments, monitoring of the plant parameters (of gene expression and/or plant tolerance to stress, growth rate, etc) can be used to determine regimen of treatment of the plant, for example, additional introduction of the nucleic acid agent of the invention, augmentation of the treatment with other treatment modalities (e.g. insecticide, antibiotics, plant hormones, etc), or in order to determine timing of fruit harvest or irrigation times. Selection of plants for monitoring in a crop or field of plants can be random or systematic (for example, sentinel plants can be pre-selected prior to the treatment).

As used herein, the phrase “plant pathogen resistance response” relates to any aspect of plant response to pathogen challenge, insult or infection, including, but not limited to microbial/pathogen-associated molecular pattern- (PAMP or MAMP) triggered immunity, or PTI, and effector-triggered immunity, or ETI, including relevant signaling cascades, molecular, morphological and physiological changes such as changes in ion-flux across the plasma membrane, phytoalexin synthesis, ROS generation, mitogen activated protein (MAP) kinase activation and protein phosphorylation, pathogenesis-related protein synthesis, callose deposition, stomatal closure, growth inhibition and hypersensitive response. The hypersensitive response includes, but is not limited to cytoplasmic shrinkage, chromatin condensation, mitochondrial swelling, vacuolization and chloroplast disruption at the site of insult (and remotely, in the systemic acquired response), resulting from down-regulation of photosynthesis, increased production and accumulation of reactive oxygen species, reactive nitrogen oxide intermediates and the defense hormones salicylic and jasmonic acid, activation of MAPK cascades changes in intracellular calcium levels and transcriptional reprogramming, and occlusion of sieve elements by callose formation and P-protein accumulation.

As used herein, the phrase “plant pathogen resistance response gene” is defined as a plant gene, the expression of which is associated with, directly or indirectly, the changes occurring in a plant in response to pathogen challenge, insult or infection. The plant pathogen resistance response gene can be a plant gene, the expression of which is altered (i.e. up-regulated or down-regulated) in response to pathogen challenge, insult or infection. The term “gene” is used broadly to refer to any segment of nucleic acid associated with a biological function. Thus, genes include coding sequences and/or the regulatory sequences required for their expression. For example, gene refers to a nucleic acid fragment that expresses mRNA or functional RNA, or encodes a specific protein, and which includes regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

Plant pathogen resistance genes include, but are not limited to genes for signaling cascade intermediates such as MAPK, jasmonic acid, salicylic acid and fatty acids, enzymes and proteins associated with ROS production, carbohydrate and energy metabolism, chloroplast- and photosynthesis related gene products, sugar polymer biosynthesis and degradation, sugar transport and export, volatile hormone biosynthesis and degradation, carbohydrate transport genes, “R” genes and the like. In some embodiments the plant pathogen response includes, but is not limited to changes in Salicylic acid levels, changes in Jasmonic acid levels, changes in Gibberellic acid levels, changes in Auxin levels, changes in Cytokinin levels, changes in Ethylene levels, changes in ABA phyto-hormones levels, up-regulation of phyto-hormone-related genes, biosynthesis, deposition and degradation of callose, reactive oxygen species production, functional FLS2/BAK1 complex formation, protein-kinase pathway activation, phloem blockage, starch accumulation, starch accumulation in phloem parenchyma cells, polymer sieve formation, changes in carbohydrate metabolism, cambial activity aberrations, sucrose accumulation and carotenoid synthesis.

As used herein, the phrase “plant pathogen resistance response gene product” refers to a product of the expression of a plant pathogen resistance response gene-including, but not limited to the RNA transcript of the plant pathogen resistance response gene and a peptide or polypeptide encoded by a sequence of a plant pathogen resistance response gene.

In some embodiments of the invention, modulating the at least one plant pathogen resistance response is achieved by reducing the expression of a plant pathogen resistance response gene. Thus, in some embodiments, the at least one plant pathogen resistance response gene is a plant gene whose expression is increased in association with the plant pathogen resistance response. Many plant pathogen resistance response genes which are up-regulated in response to pathogen challenge, insult or infection have been identified, mostly through expression profiles of diseased and healthy plants. For example, US Patent Publication 20080172765 to Kitagiri et al discloses plant genes, the expression of which is altered, either increased or decreased, in response to pathogen infection.

In one embodiment, the at least one plant pathogen resistance response gene is selected from the group consisting of an ADP-glucose pyrophosphorylase large subunit (AGPase) gene product, a glucose-6-phosphate/phosphate translocator (GPT) gene product, a Callose synthase gene product and a Myb transcriptional regulator (MYB) gene product.

Table II provides a partial list of plant genes (Arabidopsis homologues) associated with plant pathogen responses, which can be targets for reduction in expression by introducing the nucleic acid agent of the invention.

TABLE II
PLANT PATHOGEN RESISTANCE GENES-ARABIDOPSIS THALIANA
HOMOLOGUES (ARABIDOPSIS GENE SYMBOL)

| Seq | GENE | |
|---|---|---|
| ID NO: | SYMBOL | |
| 1 | AT1G69530.3 | member of alpha-expansin gene family |
| 2 | AT1G70710.1 | endo-1,4-beta-glucanase |
| 3 | AT3G11980.1 | similar to fatty acid reductases |
| 4 | AT2G39700.1 | putative expansin |
| 5 | AT3G44730.1 | kinesin-like protein 1 (KP1) |
| 6 | AT4G17030.1 | expansin-like B1 (EXLB1), a member of the expansin family |
| 7 | AT5G27670.1 | HTA7, a histone H2A protein |
| 8 | AT1G08560.1 | member of SYP11 syntaxin Gene Family |
| 9 | AT5G41040.1 | feruloyl-CoA transferase |
| 10 | AT1G08880.1 | HTA5, a histone H2A protein |
| 11 | AT1G09200.1 | histone superfamily protein |
| 12 | AT4G20780.1 | calcium sensor (calmoduline like 42) |
| 13 | AT1G02360.1 | chitinase family protein |
| 14 | AT1G72150.1 | novel cell-plate-associated protein (PATL1 or Patellin 1) |
| 15 | AT1G71692.1 | member of the MADS box family of transcription factors |
| 16 | AT5G59030.1 | putative copper transport protein (copper transporter 1) |
| 17 | AT1G49320.1 | USPL1 (unknown seed protein like 1) |
| 18 | ATCG00830.1 | ribosomal protein L2 |
| 19 | AT2G02780.1 | leucine-rich repeat protein kinase family protein |
| 20 | AT1G24430.1 | HXXXD-type acyl-transferase family protein |
| 21 | AT1G58170.1 | disease resistance-responsive (dirigent-like protein) family protein |
| 22 | AT3G07990.1 | serine carboxypeptidase-like 27 (SCPL27) |
| 23 | AT3G04920.1 | ribosomal protein S24e family protein |
| 24 | AT3G49340.1 | cysteine proteinases superfamily protein |
| 25 | AT5G48740.1 | leucine-rich repeat protein kinase family protein |
| 26 | AT2G04160.1 | protein similar to subtilisin-like serine protease |
| 27 | AT2G05920.1 | subtilase family protein |
| 28 | AT1G04120.1 | member of MRP subfamily/ABC transporter subfamily C |
| 29 | AT1G08830.1 | cytosolic copper/zinc superoxide dismutase CSD1 |
| 30 | AT3G17390.1 | S-adenosylmethionine synthetase |
| 31 | AT1G75040.1 | thaumatin-like protein (PR5) |
| 32 | AT3G45140.1 | chloroplast lipoxygenase (lipoxygenase 2) |
| 33 | AT5G33340.1 | protein with aspartic protease activity (CDR1, constitutive disease |
| | | resistance 1) |
| 34 | AT5G13930.1 | chalcone synthase (CHS) |
| 35 | AT1G23740.1 | oxidoreductase, zinc-binding dehydrogenase family protein |
| 36 | AT2G37040.1 | phenylalanine ammonia-lyase (PAL1) |
| 37 | AT3G46970.1 | cytosolic alpha-glucan phosphorylase |
| 38 | AT5G24090.1 | chitinase A (class III) |
| 39 | AT2G21050.1 | member of the AUX1 LAX family of auxin influx carriers (LAX2) |
| 40 | AT5G02140.1 | pathogenesis-related thaumatin superfamily protein |
| 41 | AT1G19670.1 | chlorophyllase |
| 42 | AT1G76690.1 | 12-oxophytodienoic acid reductases |
| 43 | AT3G54420.1 | EP3 chitinase |
| 44 | AT1G15010.1 | unknown protein |
| 45 | AT1G76680.2 | member of an alpha/beta barrel fold family of FMN-containing |
| | | oxidoreductases |
| 46 | AT2G02990.1 | member of the ribonuclease T2 family |
| 47 | AT4G33420.1 | peroxidase superfamily protein |
| 48 | AT5G05340.1 | peroxidase superfamily protein |
| 49 | AT4G11290.1 | peroxidase superfamily protein |
| 50 | AT2G36570.1 | leucine-rich repeat protein kinase family protein |
| 51 | AT5G06800.1 | Myb-like HTH transcriptional regulator family protein |
| 52 | AT4G08250.1 | GRAS family transcription factor |
| 53 | AT3G54320.1 | transcription factor of the AP2/ERWEBP class |
| 54 | AT1G61800.1 | glucose-6-phosphate/phosphate transporter 2 |
| 55 | AT1G05300.1 | member of Fe(II) transporter isolog family (ZIP5, zinc transporter 5 |
| | | precursor) |
| 56 | AT2G37870.1 | bifunctional inhibitor/lipid-transfer protein/seed storage 2S albumin |
| | | superfamily protein |
| 57 | AT3G13080.1 | ATP-dependent MRP-like ABC transporter |
| 58 | AT1G71050.1 | heavy metal transport/detoxification superfamily protein |
| 59 | AT3G18280.1 | bifunctional inhibitor/lipid-transfer protein/seed storage 2S albumin |
| | | superfamily protein |
| 60 | AT5G59030.1 | putative copper transport protein (copper transporter 1) |
| 61 | AT5G19600.1 | sulfate transporter Sultr3; 5. |
| 62 | AT1G69870.1 | low affinity nitrate transporter NRT1.7 |
| 63 | AT5G44560.1 | VPS2.2 |
| 64 | AT3G24450.1 | heavy metal transport/detoxification superfamily protein |
| 65 | AT1G10970.1 | member of Zrt- and Irt-related protein (ZIP) family |
| 66 | AT1G72160.1 | sec14p-like phosphatidylinositol transfer family protein |
| 67 | AT3G22910.1 | ATPase E1-E2 type family protein/haloacid dehalogenase-like |
| | | hydrolase family protein |
| 68 | AT1G30690.1 | sec14p-like phosphatidylinositol transfer family protein |
| 69 | AT5G65980.1 | auxin efflux carrier family protein |
| 70 | AT3G51670.1 | SEC14 cytosolic factor family protein/phosphoglyceride transfer |
| | | family protein |
| 71 | AT2G14580.1 | pathogenesis related protein (basic PR1-like protein) |
| 72 | AT5G51920.1 | pyridoxal phosphate (PLP)-dependent transferases superfamily |
| | | protein |
| 73 | AT4G03620.1 | myosin heavy chain-related |
| 74 | AT4G25000.1 | secreted protein involved in starch mobilization |
| 75 | AT3G02885.1 | GAST1 protein homolog 5 (GASA5) |
| 76 | AT4G39230.1 | similar to phenylcoumaran benzylic ether reductase (PCBER) |
| 77 | AT2G18420.1 | gibberellin-regulated GASA/GAST/Snakin family protein |
| 78 | AT1G14520.1 | MIOX1 (myo-inositol oxygenase) |
| 79 | AT4G15800.1 | similar to tobacco rapid alkalinization factor (RALF) |
| 80 | AT3G08860.1 | protein with beta-alanine aminotransferase activity |
| 81 | AT2G44740.1 | cyclin p4; 1 (CYCP4; 1); |
| 82 | AT3G11520.1 | B-type mitotic cyclin |
| 83 | AT5G67070.1 | similar to tobacco rapid alkalinization factor (RALF) |
| 84 | AT3G47800.1 | galactose mutarotase-like superfamily protein |
| 85 | AT3G19000.1 | 2-oxoglutarate (2OG) and Fe(II)-dependent oxygenase superfamily |
| | | protein |
| 86 | AT3G44830.1 | lecithin: cholesterol acyltransferase family protein |
| 87 | AT5G23960.1 | sesquiterpene synthase |
| 88 | AT4G39210.1 | large subunit of ADP-glucose pyrophosphorylase |
| 89 | AT3G13610.1 | 2-oxoglutarate (2OG) and Fe(II)-dependent oxygenase superfamily |
| | | protein |
| 90 | AT5G66150.1 | glycosyl hydrolase family 38 protein |
| 91 | AT1G60470.1 | galactinol synthase 4 (GolS4) |
| 92 | AT1G09400.1 | FMN-linked oxidoreductases superfamily protein |
| 93 | AT5G15140.1 | galactose mutarotase-like superfamily protein |
| 94 | AT1G73010.1 | PPsPase1, a pyrophosphate-specific phosphatase (phosphate |
| | | starvation-induced gene 2 or PS2) |
| 95 | AT5G36160.1 | tyrosine transaminase family protein |
| 96 | AT1G17710.1 | pyridoxal phosphate phosphatase-related protein |
| 97 | AT3G22360.1 | alternative oxidase (AOX1B) |
| 98 | AT1G07180.1 | internal mitochondrial NAD(P)H dehydrogenase |
| 99 | AT5G33370.1 | GDSL-like lipase/acylhydrolase superfamily protein |
| 100 | AT1G21850.1 | SKU5 similar 8 (sks8) |
| 101 | AT1G55120.1 | protein with fructan exohydrolase (FEH) activity |
| 102 | AT1G71380.1 | cellulase 3 (CEL3) |
| 103 | AT1G73880.1 | UDP-glucosyl transferase 89B1 (UGT89B1) |
| 104 | AT5G20870.1 | O-glycosyl hydrolases family 17 protein |
| 105 | AT2G29980.1 | endoplasmic reticulum enzyme responsible for the synthesis of |
| | | 18:3 fatty acids from phospholipids |
| 106 | AT5G22810.1 | GDSL-like lipase/acylhydrolase superfamily protein |
| 107 | AT1G33720.1 | member of CYP76C (cytochrome P450) |
| 108 | AT5G56970.1 | similar to cytokinin oxidase/dehydrogenase (cytokinin oxidase 3) |
| 109 | AT1G41830.1 | SKU5-similar 6 (SKS6) |
| 110 | AT4G31950.1 | member of CYP82C (cytochrome P450) |
| 111 | AT1G22380.1 | putative UDP-glucosyl transferase |
| 112 | AT3G57270.1 | member of glycosyl hydrolase family 17 beta-1,3-glucanase 1) |
| 113 | AT3G03350.2 | NAD(P)-binding Rossmann-fold superfamily protein |
| 114 | AT1G73370.1 | protein with sucrose synthase activity (SUS6) |
| 115 | AT1G22650.1 | plant neutral invertase family protein |
| 116 | AT5G13870.1 | endoxyloglucan transferase EXGT-A4 |
| 117 | AT4G38540.1 | FAD/NAD(P)-binding oxidoreductase family protein |
| 118 | AT2G37700.1 | fatty acid hydroxylase superfamily |
| 119 | AT5G36110.1 | member of CYP716A, cytochrome P450 |
| 120 | AT1G09155.1 | phloem protein 2-B15 (PP2-B15) |
| 121 | AT1G12030.1 | unknown protein |
| 122 | AT1G17860.1 | Kunitz family trypsin and protease inhibitor protein |
| 123 | AT4G35150.1 | O-methyltransferase family protein |
| 124 | AT4G36850.1 | PQ-loop repeat family protein/transmembrane family protein |
| 125 | AT3G43110.1 | unknown protein |
| 126 | AT5G02580.1 | protein 1589 of unknown function |
| 127 | AT5G62360.1 | invertase/pectin methylesterase inhibitor superfamily protein |
| 128 | AT1G63310.1 | unknown protein |
| 129 | AT5G66430.1 | S-adenosyl-L-methionine-dependent methyltransferases |
| | | superfamily protein |
| 130 | AT2G18660.1 | PNP-A (Plant Natriuretic Peptide A) |
| 131 | AT3G01670.1 | unknown protein |
| 132 | AT3G20570.1 | early nodulin-like protein 9 (ENODL9) |
| 133 | AT1G55290.1 | similar to oxidoreductase, 2OG-Fe(II) oxygenase |
| 134 | AT5G04010.1 | F-box family protein |
| 135 | AT5G67140.1 | F-box/RNI-like superfamily protein |
| 136 | AT3G01680.1 | protein with Mediator complex subunit Med28 |
| 137 | AT2G32280.1 | protein of unknown function (DUF1218) |
| 138 | AT3G42725.1 | putative membrane lipoprotein |
| 139 | AT5G39050.1 | HXXXD-type acyl-transferase family protein |
| 140 | AT3G15650.2 | alpha/beta-Hydrolases superfamily protein |
| 141 | AT2G41200.1 | unknown protein |
| 142 | AT2G42820.1 | HVA22-like protein F (HVA22F) |
| 143 | AT2G47780.1 | rubber elongation factor protein (REF) |
| 144 | AT3G17380.1 | TRAF-like family protein |
| 145 | AT4G35160.1 | O-methyltransferase family protein |
| 146 | AT4G31840.1 | early nodulin-like protein 15 (ENODL15) |
| 147 | AT3G17350.1 | unknown protein |
| 148 | AT3G50930.1 | cytochrome BC1 synthesis (BCS1) |
| 149 | AT5G22740.1 | beta-mannan synthase |
| 150 | AT3G54040.1 | PAR1 protein |
| 151 | AT2G46490.1 | unknown protein |
| 152 | AT5G66920.1 | SKU5 similar 17 (sks17) |
| 153 | AT2G03430.1 | ankyrin repeat family protein |
| 154 | AT2G32300.1 | uclacyanin 1 |
| 155 | AT4G24780.1 | pectin lyase-like superfamily protein |
| 156 | AT5G23660.1 | homolog of the Medicago nodulin MTN3 |
| 157 | AT5G16250.1 | unknown protein |
| 158 | AT4G27900.1 | CCT motif family protein |
| 159 | AT3G01680.1 | unknown protein |
| 160 | AT3G59940.1 | galactose oxidase/kelch repeat superfamily protein |
| 161 | AT1G70470.1 | unknown protein |
| 162 | AT2G32280.1 | unknown protein |
| 163 | AT2G25737.1 | sulfite exporter TauE/SafE family protein |
| 164 | AT2G01610.1 | plant invertase/pectin methylesterase inhibitor superfamily protein |
| 165 | AT5G62350.1 | plant invertase/pectin methylesterase inhibitor superfamily protein |
| 166 | AT2G25625.1 | unknown protein |
| 167 | AT1G60390.1 | polygalacturonase 1 (PG1) |
| 168 | AT1G72000.1 | plant neutral invertase family protein |
| 169 | AT5G05270.1 | chalcone-flavanone isomerase family protein |
| 170 | AT4G31590.1 | similar to cellulose synthase |
| 171 | AT5G35740.1 | carbohydrate-binding X8 domain superfamily protein |
| 172 | AT2G46630.1 | unknown protein |
| 173 | AT5G44400.1 | FAD-binding berberine family protein |
| 174 | AT1G05560.1 | UDP-GLUCOSE TRANSFERASE 1 |
| 175 | AT1G05570.1 | CALLOSE SYNTHASE 1 |
| 176 | AT1G06490.1 | GLUCAN SYNTHASE-LIKE 7 |
| 177 | AT2G13680.1 | GLUCAN SYNTHASE-LIKE 2 |
| 178 | AT2G31960.1 | GLUCAN SYNTHASE-LIKE 3 |
| 179 | AT3G14570.1 | GLUCAN SYNTHASE-LIKE 4 |
| 180 | AT3G59100.1 | GLUCAN SYNTHASE-LIKE 11 |
| 181 | AT4G03550.1 | ENHANCER OF EDR1 3 GLUCAN SYNTHASE-LIKE 5 |
| 182 | AT4G04970.1 | GLUCAN SYNTHASE LIKE 1 |
| 183 | AT5G13000.1 | CALLOSE SYNTHASE 3 |
| 184 | AT5G13000.2 | GLUCAN SYNTHASE-LIKE 12 |
| 185 | AT5G36870.1 | GLUCAN SYNTHASE-LIKE 9 |

Thus, in some embodiments, the isolated nucleic acid agent comprises a nucleic sequence which specifically reduces a plant pathogen resistance gene product having at least 60% sequence identity to any of the sequences of TABLE II. In some embodiments, the plant pathogen gene product is 60-75% identical, 70-85% identical, 80-90% identical, 90-95% identical or 100% identical to the sequence of Table II.

In some embodiments, the targeted gene products include, but are not limited to polynucleotide sequences having at least 60% identity to any of the sequences of TABLE III. In some embodiments, the plant pathogen gene product is 60-75% identical, 70-85% identical, 80-90% identical, 90-95% identical or 100% identical to the sequence of Table III.

TABLE III
ABBREVIATED LIST of PLANT PATHOGEN RESISTANCE
GENES-ARABIDOPSIS THALIANA HOMOLOGUES

| Seq | GENE | |
|---|---|---|
| ID NO: | SYMBOL | |
| 186 | AT1G70710.1 | endo-1,4-beta-glucanase |
| 187 | AT4G20780.1 | calcium sensor (calmoduline like 42) |
| 188 | AT5G59030.1 | putative copper transport protein (copper |
| | | transporter 1) |
| 189 | AT1G58170.1 | disease resistance-responsive (dirigent-like |
| | | protein) family protein |
| 190 | AT1G08830.1 | cytosolic copper/zinc superoxide dismutase |
| | | CSD1 |
| 191 | AT3G17390.1 | S-adenosylmethionine synthetase |
| 192 | AT3G45140.1 | chloroplast lipoxygenase (lipoxygenase 2) |
| 193 | AT5G33340.1 | protein with aspartic protease activity (CDR1, |
| | | constitutive disease resistance 1) |
| 194 | AT5G13930.1 | chalcone synthase (CHS) |
| 195 | AT5G06800.1 | Myb-like HTH transcriptional regulator family |
| | | protein |
| 196 | AT1G61800.1 | glucose-6-phosphate/phosphate transporter 2 |
| 197 | AT4G39210.1 | large subunit of ADP-glucose pyrophosphory- |
| | | lase |
| 198 | AT1G73880.1 | UDP-glucosyl transferase 89B1 (UGT89B1) |
| 199 | AT1G22380.1 | putative UDP-glucosyl transferase |
| 200 | AT1G09155.1 | phloem protein 2-B15 (PP2-B15) |
| 201 | AT1G17860.1 | Kunitz family trypsin and protease inhibitor |
| | | protein |
| 202 | AT5G05270.1 | chalcone-flavanone isomerase family protein |
| 203 | AT4G31590.1 | similar to cellulose synthase |

Thus, according to some embodiments of the invention there is provided an isolated nucleic acid agent comprising a nucleic acid sequence which specifically reduces the expression of at least one plant pathogen resistance gene product.

In some embodiments, the isolated nucleic acid agent comprises a nucleic sequence which specifically reduces a plant pathogen resistance gene product having at least 60%, sequence identity to any of the sequences of TABLE II.

HuangLongBing is predominately a disease of citrus and citrus-related plants and trees. Thus, in some embodiments, the isolated nucleic acid of the invention is directed to downregulation of citrus-specific gene products.

Table IV provides a partial list of citrus plant polynucleotide sequences associated with citrus plant pathogen responses, which can be targets for reduction in expression by introducing the nucleic acid agent of the invention. Table IV(a) provides a further list of candidate targets for reduction of gene expression, based on function of the specified gene-sequences in italics are citrus pathogen plant response-associated sequences, while the bolded sequences are sugar metabolism-related genes.

TABLE IV
CITRUS TARGET SEQUENCES (GenBank Accession Nos.)

| Seq ID NO: | GENE SYMBOL |
|---|---|
| 204 | DN959507 |
| 205 | CD573747 |
| 206 | CF828218 |
| 207 | CV710432 |
| 208 | CD575625 |
| 209 | CF835126 |
| 210 | CF505371 |
| 211 | CX675973 |
| 212 | CV719481 |
| 213 | CX642677 |
| 214 | CK935733 |
| 215 | CX643907 |
| 216 | CX296236 |
| 217 | CF832155 |
| 218 | CX053912 |
| 219 | CX643504 |
| 220 | CX077483 |
| 221 | DN618428 |
| 222 | CX666928 |
| 223 | CX675636 |
| 224 | CD574876 |
| 225 | CX663894 |
| 226 | CF838213 |
| 227 | CV884462 |
| 228 | CB292401 |
| 229 | DN623815 |
| 230 | CX635745 |
| 231 | CX301461 |
| 232 | CX672038 |
| 233 | CX045698 |
| 234 | C95463 |
| 235 | CX673765 |
| 236 | CB290861 |
| 237 | CN188208 |
| 238 | CF504694 |
| 239 | CX674093 |
| 240 | CK936991 |
| 241 | CF509114 |
| 242 | CX643181 |
| 243 | CX665015 |
| 244 | CX043703 |
| 245 | CX668229 |
| 246 | CX053885 |
| 247 | CX299724 |
| 248 | DR403986 |
| 249 | CX663493 |
| 250 | CX663308 |
| 251 | DN623584 |
| 252 | CK934943 |
| 253 | CX044216 |
| 254 | CX078485 |
| 255 | CX046318 |
| 256 | CX303177 |
| 257 | CF53269 |
| 258 | CX287481 |
| 259 | CK739465 |
| 260 | CX675684 |
| 261 | CF835779 |
| 262 | DN620798 |
| 263 | CX051466 |
| 264 | CF836284 |
| 265 | CX286914 |
| 266 | CF418391 |
| 267 | CX293439 |
| 268 | CX664023 |
| 269 | CV720146 |
| 270 | CF508503 |
| 271 | CN185151 |
| 272 | CF835597 |
| 273 | CV885632 |
| 274 | CX665957 |
| 275 | DN619087 |
| 276 | CF417971 |
| 277 | CX674752 |
| 278 | CX672325 |
| 279 | CX675696 |
| 280 | CB290596 |
| 281 | CX075198 |
| 282 | DN618043 |
| 283 | CV704545 |
| 284 | CX671045 |
| 285 | CF653559 |
| 286 | CX640391 |
| 287 | CX302689 |
| 288 | DN958063 |
| 289 | CB250380 |
| 290 | CX543436 |
| 291 | CF508354 |
| 292 | CN184547 |
| 293 | CF837402 |
| 294 | DT214662 |
| 295 | CF507791 |
| 296 | CV716571 |
| 297 | CN184617 |
| 298 | CX666534 |
| 299 | CX293884 |
| 300 | CF418573 |
| 301 | DN795637 |
| 302 | DN622894 |
| 303 | DN799150 |
| 304 | CX671694 |
| 305 | DN619965 |
| 306 | CX664606 |
| 307 | CX544543 |
| 308 | DN618893 |
| 309 | CV714863 |
| 310 | DN795132 |
| 311 | CX293755 |
| 312 | CF417383 |
| 313 | AU300374 |
| 314 | CX046632 |
| 315 | CX663293 |
| 316 | CK933255 |
| 317 | CV709925 |
| 318 | CX077593 |
| 319 | BQ624438 |
| 320 | CN189092 |
| 321 | CX075046 |
| 322 | CF832471 |
| 323 | CX308646 |
| 324 | CN192282 |
| 325 | CX307078 |
| 326 | C95332 |
| 327 | CX295397 |
| 328 | CN181803 |
| 329 | CX076871 |
| 330 | CN191204 |
| 331 | CV704769 |
| 332 | CF417791 |
| 333 | CF831073 |
| 334 | CX664109 |
| 335 | CK934765 |
| 336 | DN622570 |
| 337 | CX302870 |
| 338 | CX298333 |
| 339 | CN191069 |
| 340 | C22284 |
| 341 | CK936315 |
| 342 | CX665686 |
| 343 | CD576001 |
| 344 | CV885460 |
| 345 | CX674613 |
| 346 | CV707520 |
| 347 | DN623989 |
| 348 | CX641508 |
| 349 | CV718422 |
| 350 | CX669149 |
| 351 | CV708715 |
| 352 | BQ623232 |
| 353 | CF418736 |
| 354 | DN959078 |
| 355 | CV708663 |
| 356 | CX673097 |
| 357 | CX302123 |
| 358 | CN187632 |
| 359 | CX673300 |
| 360 | CK701797 |
| 361 | CX635438 |
| 362 | CX545339 |
| 363 | CX053315 |
| 364 | CV709535 |
| 365 | DN798478 |
| 366 | CB292827 |
| 367 | CX671357 |
| 368 | CX069447 |
| 369 | CX287588 |
| 370 | CX297166 |
| 371 | CK934054 |
| 372 | CB291312 |
| 373 | CX076597 |
| 374 | CX301940 |
| 375 | CX544117 |
| 376 | CK665535 |
| 377 | CX669825 |
| 378 | CX670154 |
| 379 | CX674833 |
| 380 | CX072993 |
| 381 | CX641923 |
| 382 | CV709392 |
| 383 | CK936214 |
| 384 | CK936713 |
| 385 | CX046656 |
| 386 | CN190414 |
| 387 | DN618740 |
| 388 | CX667496 |
| 389 | CK933819 |
| 390 | CX666282 |
| 391 | BQ624534 |
| 392 | CD574984 |
| 393 | CX299253 |
| 394 | U82977.1 |
| 395 | DN959563 |
| 396 | DN625629 |
| 397 | DN622423 |
| 398 | DN621393 |
| 399 | DN620655 |
| 400 | DN617955 |
| 401 | DN617601 |
| 402 | CX675719 |
| 403 | CX673052 |
| 404 | CX671909 |
| 405 | CX671908 |
| 406 | CX669790 |
| 407 | CX667860 |
| 408 | CX667462 |
| 409 | CX666073 |
| 410 | CX663867 |
| 411 | CX663363 |
| 412 | CX643067 |
| 413 | CX640562 |
| 414 | CX636575 |
| 415 | CX636360 |
| 416 | CX635683 |
| 417 | CX546685 |
| 418 | CX545718 |
| 419 | CX544442 |
| 420 | CX543942 |
| 421 | CX543689 |
| 422 | CX542606 |
| 423 | CX309167 |
| 424 | CX308494 |
| 425 | CX307626 |
| 426 | CX307101 |
| 427 | CX306527 |
| 428 | CX305853 |
| 429 | CX304999 |
| 430 | CX303309 |
| 431 | CX302610 |
| 432 | CX301947 |
| 433 | CX297896 |
| 434 | CX296140 |
| 435 | CX296097 |
| 436 | CX295546 |
| 437 | CX290706 |
| 438 | CX289600 |
| 439 | CX288823 |
| 440 | CX287363 |
| 441 | CK078233 |
| 442 | CX078190 |
| 443 | CX076996 |
| 444 | CX070279 |
| 445 | CX048605 |
| 446 | CX048208 |
| 447 | CX047376 |
| 448 | CX046977 |
| 449 | CX046019 |
| 450 | CX043864 |
| 451 | CV887291 |
| 452 | CV886686 |
| 453 | CV886073 |
| 454 | CV884354 |
| 455 | CV720075 |
| 456 | CV719759 |
| 457 | CV719483 |
| 458 | CV719426 |
| 459 | CV717016 |
| 460 | CV716643 |
| 461 | CV714792 |
| 462 | CV714551 |
| 463 | CV714253 |
| 464 | CV713804 |
| 465 | CV713122 |
| 466 | CV713085 |
| 467 | CV711700 |
| 468 | CV710341 |
| 469 | CV709126 |
| 470 | CV707867 |
| 471 | CV706656 |
| 472 | CV705672 |
| 473 | CV705217 |
| 474 | CV704366 |
| 475 | CN191865 |
| 476 | CN186090 |
| 477 | CN185887 |
| 478 | CN182557 |
| 479 | CN182178 |
| 480 | CK939832 |
| 481 | CK939153 |
| 482 | CK938892 |
| 483 | CK938479 |
| 484 | CK936961 |
| 485 | CK933484 |
| 486 | CK702328 |
| 487 | CF836843 |
| 488 | CF836817 |
| 489 | CF835056 |
| 490 | CF833518 |
| 491 | CF418886 |
| 492 | CF418485 |
| 493 | CD576435 |
| 494 | CD576111 |
| 495 | CD575905 |
| 496 | CD575735 |
| 497 | CD575303 |
| 498 | CD575247 |
| 499 | CD574599 |
| 500 | CD573799 |
| 501 | CB611071 |
| 502 | CB322174 |
| 503 | CB305061 |
| 504 | CB291815 |
| 505 | CB291722 |
| 506 | CB291657 |

TABLE IV(a)

| | SEQ | |
|---|---|---|
| Gene Symb. | ID NO | |
| CX303072 | 623 | Beta-glucosidase-like |
| DN622894 | 624 | ADP-glucose pyrophosphorylase |
| DN625620 | 625 | Glucose-6-phosphate dehydrogenase |
| CB292132 | 626 | Granule-ground starch synthase |
| CX637561 | 627 | Putative UDP-glucuronosyltransferase |
| DT214451 | 628 | Beta-amylase |
| CX046632 | 629 | Extracellular acid invertase |
| CX070113 | 630 | Starch branching enzyme |
| CB292174 | 631 | Sugar transport protein |
| CX045485 | 632 | Hexose transporter |
| CX665157 | 633 | Galactose oxidase |
| CX639454 | 634 | Plant glycogenin-like starch initiation |
| | | protein |
| CX663848 | 635 | Glycosyl transferase |
| CV705038 | 636 | 4-alpha-galacturonosyltransferase |
| CF831824 | 637 | Trehalose-phosphatase |
| CK938541 | 638 | Galacturonosyltransferase |
| CX294095 | 639 | Glycosyl hydrolase |
| CN187456 | 640 | Trehalose-phosphatase |
| CK938256 | 641 | Mannase |
| CV886325 | 642 | Glycerol-3-phosphate dehydrogenase |
| DN617689 | 643 | Raffinose synthase |
| CF836851 | 644 | Probable alcohol degydrogenase |
| DN959139 | 645 | Trehalose-6-phosphate phosphatase |
| CX044393 | 646 | Probable short chain alcohol dehydrogenase |
| CK936380 | 647 | Alcohol acyl transferase |
| BQ623570 | 648 | Dirigent protein |
| CX309407 | 649 | Alpha-glucosidase-like |
| CX668300 | 650 | Chitinase |
| CF832155 | 651 | Acidic class II chitinase |
| CX638776 | 652 | Delta 1-pyrroline-5-carboxylate synthetase |
| CK937251 | 653 | Miraculin-like protein |
| CX303148 | 654 | Nam-like protein 11 |
| CN186431 | 655 | CTV protein |
| CX301461 | 656 | Unnamed protein product |
| DN619110 | 657 | Unknown protein |
| CX669483 | 658 | Early nodulin |
| DN618893 | 659 | Tyrosine aminotransferase |
| CX306211 | 660 | Nectarin 5 |
| CF507855 | 661 | Delta 1-pyrroline-5-carboxylate synthetase 2 |
| CF653559 | 662 | Pathogenesis-related protein PR-1 precursor |
| CK935794 | 663 | Glycolate oxidase |
| CX666928 | 664 | BURP domain-containing protein |
| CX305834 | 665 | Seed-specific protein |
| CX640129 | 666 | Glutaredoxin-like protein |
| CK935883 | 667 | Disease resistance-responsive protein |
| CX045772 | 668 | Putative cell death associated protein |
| CN191283 | 669 | NAM-like protein |
| CK934775 | 670 | Disease-resistance protein |
| CX286941 | 671 | Nodulin-like |
| CX301618 | 672 | Dehydration-responsive protein RD22 |
| CX296222 | 673 | Blight-associated protein p12 precursor |
| CX641603 | 674 | Disease-resistance protein |
| DR406181 | 675 | Metallothionein-like protein |
| CB293886 | 676 | NAM-like protein |
| CF835337 | 677 | Pathogenesis-related protein 4A |
| CX048331 | 678 | Putative ripening-related protein |
| CX637285 | 679 | Pathogenesis-related protein 4A |
| CX044399 | 680 | NBS-LRR resistance-like protein RGC359 |
| DN618428 | 681 | BURP-domain containing protein |
| CX545242 | 682 | Berberine bridge enzyme-like protein |
| AU186381 | 683 | Acidic class II chitinase |
| DN958104 | 684 | Beta-cyanoalanine synthase |
| CX076066 | 685 | Resistance protein candidate RGC2J |
| CF835944 | 686 | MLO protein (mildew resistance locus) |
| CX671223 | 687 | Chitinase |
| AU300664 | 688 | Pathogenesis-related protein |
| CF832471 | 689 | Multi-copper oxidase |
| DN795254 | 690 | Putative calmodulin-binding protein |
| CX292843 | 691 | Photoassimilate-responsive protein |
| CF507442 | 692 | Nodulin-like protein |
| CX305678 | 693 | Basic chitinase |
| CX048700 | 694 | Leucine-rich repeat protein |
| DN625052 | 695 | Disease resistance RGA3 protein |
| CX070975 | 696 | Small MW heat shock protein |
| CX671683 | 697 | Nodulin-like protein |
| DN618117 | 698 | NBS-LRR type disease resistance protein |
| CX045895 | 699 | Chitinase |
| CX291159 | 700 | Elicitor-inducible cytochrome P450 |
| CF833037 | 701 | Putative salicylate monooxygenase |
| CF829440 | 702 | Pectate lyase |
| CV709277 | 703 | Phosphoesterase family protein |
| CX069929 | 704 | Abl interactor-like protein-1 |
| CX643843 | 705 | Putative BURP domain containing protein |
| CX293287 | 706 | Putative nodulin protein |
| CO913159 | 707 | Glyoxal oxidase related |
| CF838393 | 708 | Chitinase class II precursor |
| CX637639 | 709 | Chitinase |
| CK936056 | 710 | Subtilisin-like protease |
| CF417485 | 711 | ENSP-like protein |
| CX296119 | 712 | Immediate-early fungal elicitor protein CMPG1 |
| CN181971 | 713 | Disease resistance LRR family protein |
| CX076036 | 714 | Avr9 Cf-9 rapidly elicited protein 111B |

In some embodiments, the nucleic acid agent of the invention is directed towards any one or more subset of the sequences of Tables IV and IV(a). Table V provides an exemplary subset of citrus plant polynucleotide sequences associated with citrus plant pathogen responses, which are also suitable targets for reduction in expression by introducing the nucleic acid agent and methods of the invention.

TABLE V
ABBREVIATED LIST OF CITRUS TARGET
SEQUENCES (GenBank Accession No.)

| | GENE |
|---|---|
| Seq ID NO: | SYMBOL |
| 205 | CD573747 |
| 216 | CX296236 |
| 220 | CX077483 |
| 226 | CF838213 |
| 231 | CX301461 |
| 235 | CX673765 |
| 236 | CB290861 |
| 238 | CF504694 |
| 239 | CX674093 |
| 240 | CK936991 |
| 258 | CX287481 |
| 261 | CF835779 |
| 263 | CX051466 |
| 269 | CV720146 |
| 302 | DN622894 |
| 324 | CN192282 |
| 333 | CF831073 |
| 335 | CK934765 |
| 339 | CN191069 |
| 379 | CX674833 |
| 389 | CK933819 |
| 390 | CX666282 |

In some embodiments, the isolated nucleic acid agent comprises a nucleic acid sequence which specifically reduces any of plant pathogen resistance gene products of the sequences of TABLES IV and IV(a). In other embodiments, the isolated nucleic acid agent comprises a nucleic acid sequence which specifically reduces any of plant pathogen resistance gene products of the sequences of TABLE V.

In some embodiments, the isolated nucleic acid agent comprises a nucleic acid sequence which specifically reduces the gene products of a gene selected from the group consisting of the AGPase gene, the GPT gene, the Callose synthase gene, the Lipoxygenase D gene, the Myb gene and the PP2-B-15 gene. In some embodiments, the AGPase gene product is encoded by SEQ ID NO: 527, or a portion thereof, the GPT gene product is encoded by SEQ ID NO: 529 or a portion thereof, the Callose synthase gene product is encoded by SEQ ID NO: 531 or a portion thereof, the Lipoxygenase D gene product is encoded by SEQ ID NO: 533 or a portion thereof, the Myb gene product is encoded by SEQ ID NO: 535 or a portion thereof, and the PP2 gene product is encoded by SEQ ID NO: 537 or a portion thereof.

In some embodiments, the isolated nucleic acid agent comprises a nucleic acid sequence comprising a nucleic acid sequence complementary to a portion of the nucleic acid sequence of the gene product of any one of the AGPase gene, the GPT gene, the Callose synthase gene, the Lipoxygenase D gene, and the Myb gene, which specifically reduces the gene products of the corresponding gene. In some embodiments, the nucleic acid sequence targeting the AGPase gene comprises SEQ ID NO: 528, or a portion thereof, the nucleic acid sequence targeting the GPT gene comprises SEQ ID NO: 530, or a portion thereof, the nucleic acid sequence targeting the Callose synthase gene comprises SEQ ID NO: 532, or a portion thereof, the nucleic acid sequence targeting the Lipoxygenase D gene comprises SEQ ID NO: 534, or a portion thereof, the nucleic acid sequence targeting the Myb gene comprises SEQ ID NO: 536, or a portion thereof and the nucleic acid sequence targeting the PP2 gene comprises SEQ ID NO: 538, or a portion thereof.

In some embodiments of aspects of the invention, the nucleic acid agent is a double stranded RNA (dsRNA). As used herein the term “dsRNA” relates to two strands of anti-parallel polyribonucleic acids held together by base pairing. The two strands can be of identical length or of different lengths provided there is enough sequence homology between the two strands that a double stranded structure is formed with at least 80%, 90%, 95% or 100% complementarity over the entire length. According to an embodiment of the invention, there are no overhangs for the dsRNA molecule. According to another embodiment of the invention, the dsRNA molecule comprises overhangs. According to other embodiments, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed.

It will be noted that the dsRNA can be defined in terms of the nucleic acid sequence of the DNA encoding the target gene transcript, and it is understood that a dsRNA sequence corresponding to the coding sequence of a gene comprises an RNA complement of the gene's coding sequence, or other sequence of the gene which is transcribed into RNA.

Thus, in some embodiments, the isolated nucleic acid agent comprises a nucleic sequence which is complementary to a nucleic acid sequence having at least 60% sequence identity to any of the sequences of TABLE II or TABLE III. In some embodiments, the nucleic acid sequence is 60-75% identical, 70-85% identical, 80-90% identical, 90-95% identical or 100% identical to the sequence of Table II or III.

In other embodiments, wherein the plant or tree is a citrus or citrus-related plant or tree, the isolated nucleic acid agent comprises a nucleic sequence complementary to any of the polynucleotide sequences of TABLES IV and IV(a). In still other embodiments, the plant is a citrus or citrus-related plant or tree and the isolated nucleic acid agent comprises a nucleic sequence which is complementary to any of the polynucleotide sequences of TABLE V.

The inhibitory RNA sequence can be greater than 90% identical, or even 100% identical, to the portion of the target gene transcript. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 60 degrees C. hybridization for 12-16 hours; followed by washing). The length of the double-stranded nucleotide sequences complementary to the target gene transcript may be at least about 18, 19, 21, 25, 50, 100, 200, 300, 400, 491, 500, or more bases. In some embodiments of some aspects of the invention, the length of the double-stranded nucleotide sequence is approximately from about 18 to about 510 nucleotides in length for genes of Citrus spp. Such as, but not limited to sweet oranges: Citrus sinensis, lemons: Citrus limon and sour orange: Citrus aurantium.

The term “corresponds to” as used herein means a polynucleotide sequence homologous to all or a portion of a reference polynucleotide sequence. In contradistinction, the term “complementary to” is used herein to mean that the complementary sequence is homologous to all or a portion of a reference polynucleotide sequence. For example, the nucleotide sequence “TATAC” corresponds to a reference sequence “TATAC” and is complementary to a reference sequence “GTATA”.

The present teachings relate to various lengths of dsRNA, whereby the shorter version i.e., x is shorter or equals 50 bp (e.g., 17-50), is referred to as siRNA or miRNA. Longer dsRNA molecules of 51-600 are referred to herein as dsRNA, which can be further processed for siRNA molecules. According to some embodiments, the nucleic acid sequence of the dsRNA is greater than 15 base pairs in length. According to yet other embodiments, the nucleic acid sequence of the dsRNA is 19-25 base pairs in length, 30-100 base pairs in length, 100-250 base pairs in length or 100-500 base pairs in length. According to still other embodiments, the dsRNA is 300-600 base pairs in length, 350-500 base pairs in length or 400-450 base pairs in length. In some embodiments, the dsRNA is 400 base pairs in length.

The term “siRNA” refers to small inhibitory RNA duplexes (generally between 17-30 basepairs, but also longer e.g., 31-50 bp) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3 (Brummelkamp, T. R. et al. (2002) Science 296: 550, SEQ ID NO: 517) and 5′-UUUGUGUAG-3′ (Castanotto, D. et al. (2002) RNA 8:1454, SEQ ID NO: 518). It will be recognized by one of skill in the art that the resulting single chain oligonucleotide forms a stem-loop or hairpin structure comprising a double-stranded region capable of interacting with the RNAi machinery.

As used herein, the phrase “microRNA (also referred to herein interchangeably as “miRNA” or “miR”) or a precursor thereof” refers to a microRNA (miRNA) molecule acting as a post-transcriptional regulator. Typically, the miRNA molecules are RNA molecules of about 20 to 22 nucleotides in length which can be loaded into a RISC complex and which direct the cleavage of another RNA molecule, wherein the other RNA molecule comprises a nucleotide sequence essentially complementary to the nucleotide sequence of the miRNA molecule.

Typically, a miRNA molecule is processed from a “pre-miRNA” or as used herein a precursor of a pre-miRNA molecule by proteins, such as DCL proteins, present in any plant cell and loaded onto a RISC complex where it can guide the cleavage of the target RNA molecules.

Pre-microRNA molecules are typically processed from pri-microRNA molecules (primary transcripts). The single stranded RNA segments flanking the pre-microRNA are important for processing of the pri-miRNA into the pre-miRNA. The cleavage site appears to be determined by the distance from the stem-ssRNA junction (Han et al. 2006, Cell 125, 887-901, 887-901).

As used herein, a “pre-miRNA” molecule is an RNA molecule of about 100 to about 200 nucleotides, preferably about 100 to about 130 nucleotides which can adopt a secondary structure comprising an imperfect double stranded RNA stem and a single stranded RNA loop (also referred to as “hairpin”) and further comprising the nucleotide sequence of the miRNA (and its complement sequence) in the double stranded RNA stem. According to a specific embodiment, the miRNA and its complement are located about 10 to about 20 nucleotides from the free ends of the miRNA double stranded RNA stem. The length and sequence of the single stranded loop region are not critical and may vary considerably, e.g. between 30 and 50 nucleotides in length. The complementarity between the miRNA and its complement need not be perfect and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand which at its 5′ end is the least involved in hydrogen bonding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule. As is known in the art, bonding between A and U involving two hydrogen bonds, or G and U involving two hydrogen bonds is less strong that between G and C involving three hydrogen bonds.

Naturally occurring miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds. Some pre-miRNA scaffolds may be preferred over others for their efficiency to be correctly processed into the designed microRNAs, particularly when expressed as a chimeric gene wherein other DNA regions, such as untranslated leader sequences or transcription termination and polyadenylation regions are incorporated in the primary transcript in addition to the pre-microRNA.

In some embodiments, the nucleic acid agent is a hairpin RNA (hpRNA) interference or intron-containing hairpin RNA (ihpRNA) interference construct. Methods for gene silencing in plants using hairpin RNA vectors are well known in the art and considered efficient at inhibiting the gene expression in plants. See, for example, Waterhouse and Helliwell (2003) Nat. Rev. Genet. 4:29-38 and Yan et al, PLoS one (2012) 7:e38186.

For hpRNA silencing, the expression cassette is designed to express an RNA molecule that hybridizes with itself to form a hairpin structure that includes a single-stranded loop region and a base-paired stem. The base-paired stem region includes a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference for silencing. hpRNA molecules are considered efficient at inhibiting and silencing gene expression, and the RNA interference they induce may be inherited by subsequent plant generations. Methods for using hpRNA interference to inhibit or silence the expression of genes are described, for example, in US Patent Publication No. 2010058490 to Waterhouse et al. A transient assay for the efficiency of hpRNA constructs to silence gene expression in vivo has been described by Panstruga et al. (2003) Mol. Biol. Rep. 30:135-150.

For ihpRNA, the silencing molecules have the same general structure as for hpRNA, but the RNA molecule additionally includes an intron that is capable of being spliced in the cell in which the ihpRNA is expressed. The use of an intron minimizes the loop size in the hairpin RNA molecule following splicing, and this increases the interference efficiency.

According to the present teachings, the dsRNA molecules may be naturally occurring or synthetic.

The dsRNA can be a mixture of long and short dsRNA molecules such as, dsRNA, siRNA, siRNA+dsRNA, siRNA+miRNA, hpRNA or a combination of same. According to a specific embodiment, the dsRNA is an siRNA (100%).

The dsRNA molecule is designed for specifically targeting a target gene of interest. It will be appreciated that the dsRNA can be used to down-regulate one or more target genes. If a number of target genes are targeted, a heterogenic composition which comprises a plurality of dsRNA molecules for targeting a number of target genes is used. Alternatively said plurality of dsRNA molecules are separately applied to the seeds (but not as a single composition). According to a specific embodiment, a number of distinct dsRNA molecules for a single target are used, which may be separately or simultaneously (i.e., co-formulation) applied.

According to an embodiment of the invention, the target gene is endogenous to the plant. Downregulating such a gene is typically important for conferring the plant with an improved, agricultural, horticultural, nutritional trait (“improvement” or an “increase” is further defined herein).

As used herein “endogenous” refers to a gene which expression (mRNA or protein) takes place in the plant. Typically, the endogenous gene is naturally expressed in the plant or originates from the plant. Thus, the plant may be a wild-type plant. However, the plant may also be a genetically modified plant (transgenic).

Downregulation of the target gene may be important for conferring improved one of—, or at least one of (e.g., two of— or more), biomass, vigor, yield, fruit quality, abiotic and/or biotic stress tolerance or improved nitrogen use efficiency.

In some embodiments, target genes for downregulation by the methods and nucleic acid agents of the present invention are plant pathogen resistance gene products which expression thereof is upregulated following infection of the plant with a plant pathogen, for example, the plant pathogen of the plant infection (e.g. Candidatus Liberibacter spp).

Exemplary target genes include, but are not limited to, genes for signaling cascade intermediates such as MAPK, jasmonic acid, salicylic acid and fatty acids, enzymes and proteins associated with ROS production, carbohydrate and energy metabolism, chloroplast- and photosynthesis related gene products and the like, sugar polymer biosynthesis and degradation, sugar transport and export, volatile hormone biosynthesis and degradation, carbohydrate transport, ‘R’ genes, which expression can be silenced to improve the yield, growth rate, vigor, biomass, fruit quality or stress tolerance of a plant when infected with a plant pathogen. Other examples of target genes which may be subject to modulation according to the present teachings are described herein.

In some embodiments, target genes include, but are not limited to genes for pathogen resistance response such as changes in Salicylic acid levels, changes in Jasmonic acid levels, changes in Gibberellic acid levels, changes in Auxin levels, changes in Cytokinin levels, changes in Ethylene levels, changes in ABA phyto-hormones levels, up-regulation of phyto-hormone-related genes, biosynthesis, deposition and degradation of callose, reactive oxygen species production, functional FLS2/BAK1 complex formation, protein-kinase pathway activation, phloem blockage, starch accumulation, starch accumulation in phloem parenchyma cells, polymer sieve formation, changes in carbohydrate metabolism, cambial activity aberrations, sucrose accumulation and carotenoid synthesis.

The target gene may comprise a nucleic acid sequence which is transcribed to an mRNA which codes for a polypeptide.

Alternatively, the target gene can be a non-coding gene such as a miRNA or a siRNA.

For example, in order to silence the expression of an mRNA of interest, synthesis of the dsRNA suitable for use with some embodiments of the invention can be selected as follows. First, the mRNA sequence is scanned including the 3 UTR and the 5′ UTR.

Second, the mRNA sequence is compared to an appropriate genomic database using any sequence alignment software, such as the BLAST software available from the NCBI server (wwwdotncbidotnlmdotnihdotgov/BLAST/). Putative regions in the mRNA sequence which exhibit significant homology to other coding sequences are filtered out.

Qualifying target sequences are selected as template for dsRNA synthesis. Preferred sequences are those that have as little homology to other genes in the genome to reduce an “off-target” effect.

It will be appreciated that the RNA silencing agent of some embodiments of the invention need not be limited to those molecules containing only RNA, but further encompasses chemically-modified nucleotides and non-nucleotides.

The dsRNA may be synthesized using any method known in the art, including either enzymatic syntheses or solid-phase syntheses. These are especially useful in the case of short polynucleotide sequences with or without modifications as explained above. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

According to a specific embodiment, the nucleic acid agent is provided to the plant in a configuration devoid of a heterologous promoter for driving recombinant expression of the dsRNA (exogenous), rendering the nucleic acid molecule of the instant invention a naked molecule. The nucleic acid agent may still comprise modifications that may affect its stability and bioavailability (e.g., PNA).

The term “recombinant expression” refers to an expression from a nucleic acid construct.

As used herein “devoid of a heterologous promoter for driving expression of the dsRNA” means that the molecule doesn't include a cis-acting regulatory sequence (e.g., heterologous) transcribing the dsRNA. As used herein the term “heterologous” refers to exogenous, not-naturally occurring within a native cell of the plant (such as by position of integration, or being non-naturally found within the cell).

The nucleic acid agent can be further comprised within a nucleic acid construct comprising additional regulatory elements. Thus, according to some embodiments of aspects of the invention there is provided a nucleic acid construct comprising isolated nucleic acid agent comprising a nucleic acid sequence which specifically reduces the expression of at least one plant pathogen resistance gene product.

For transcription from a transgene in vivo or from an expression cassette, a regulatory region (e.g., promoter, enhancer, silencer, leader, intron and polyadenylation) may be used to modulate the transcription of the RNA strand (or strands). Therefore, in one embodiment, there is provided a nucleic acid construct comprising the nucleic acid agent. The nucleic acid construct can have polynucleotide sequences constructed to facilitate transcription of the RNA molecules of the present invention are operably linked to one or more promoter sequences functional in a host cell. The polynucleotide sequences may be placed under the control of an endogenous promoter normally present in the host genome. The polynucleotide sequences of the present invention, under the control of an operably linked promoter sequence, may further be flanked by additional sequences that advantageously affect its transcription and/or the stability of a resulting transcript. Such sequences are generally located upstream of the promoter and/or downstream of the 3 end of the expression construct. The term “operably linked”, as used in reference to a regulatory sequence and a structural nucleotide sequence, means that the regulatory sequence causes regulated expression of the linked structural nucleotide sequence. “Regulatory sequences” or “control elements” refer to nucleotide sequences located upstream, within, or downstream of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, termination sequences, pausing sequences, polyadenylation recognition sequences, and the like. In some embodiments, the host is a plant, and promoter and other regulatory elements are active in plants.

The nucleic acid agent can be delivered to the plants in a variety of ways. As mentioned, nucleic acids can be introduced into plants by injection, aerosol application, dusting, in a dry flowable, an emulsifiable flowable, as a granule, in a microencapsulation, in a pellet, as a soluble powder, with an injuring agent, bombardment, by air-brush spraying, supplemented with a plant hormone, added to an agar-based germination platform, supplemented with a wetting agent, supplemented with polysaccharide such as sodium alginate or chitosan, supplemented with transfection reagents. In some non-limiting embodiments, the nucleic acid agent is formulated for application by irrigation, positive or negative pressure application. In other non-limiting embodiments, the nucleic acid agent is formulated for application along with a plant nutrition supplement, for example, a urea-triazone supplement, such as the commercially available urea-triazone N (N-SURE®, Tessenderlo-Kerley, Pheonix, Ariz.). Administration of the nucleic acid with such a urea-triazone supplement is suitable for both foliar and soil application in all plants, particularly for commercial vegetable and fruit crops. The nucleic acid agent can be provided to the plant as a nucleic acid, without additional agents (e.g. transfection agents) or encapsulation or other formulation, or, optionally, formulated with additional agents for example, for enhancing uptake or efficacious downregulation of the target gene products.

In some particular embodiments, the nucleic acid agent is introduced into the plant via injection into the plant or tree. Methods suitable for injection of nucleic acids and nucleic acid agents into the plant or tree are described, for example, in Utah State University Cooperative Extension's informational paper by Michael Kuhns (NR/FF/020), including trunk implantation (see for example, Acecaps™ and Medicaps™), pressurized and no-pressure trunk injection (see, for example, Arborjet Tree IV™ and Wedgle™, Tree Tech™ and Rainbow Tree Care™), soil injection and trunk basal spray.

In other embodiments, the nucleic acid agent is introduced into the plant using virus-induced gene silencing. Virus-induced gene silencing (VIGS) offers an attractive alternative to transgenic technology as it allows the investigation of gene functions without plant transformation (Ruiz et al., 1998; Burch-Smith et al., 2004). A partial fragment of a candidate gene is inserted into the virus vector to generate a recombinant virus. Infection (e.g. via Agrobacterium) of plants with this recombinant virus leads to the production of virus-related small interfering RNAs (siRNAs) (Baulcombe, 2004), which can mediate degradation of related endogenous gene transcripts, resulting in silencing of the candidate gene expression in inoculated plants (Brigneti et al., 2004; Burch-Smith et al., 2004). The silencing effect on endogenous gene expression can usually be assayed 1-2 weeks after virus inoculation. VIGS has become one of the most widely used and indeed important reverse genetics tools, especially for non-model plants. In some embodiments, candidate nucleic acid sequences for specifically reducing the expression of plant pathogen resistance gene products are screened in model infections and/or field conditions using VIGS.

VIGS can be used for silencing or reducing expression of candidate plant-pathogen related genes. Using viral vectors to silence an endogenous plant gene may involve cloning into the viral genome, without significantly compromising viral replication and movement, a nucleotide fragment sharing a certain percentage identity or complementarity to the endogenous plant gene. The principle and detailed protocol regarding the VIGS system have been described (Dinesh-Kumar, et al., (2003) Methods in Mol. Biol. 236:287-94; Lu, et al., (2003) Methods 30:296-303). Several different RNA and DNA plant viruses have been modified to serve as vectors for gene expression. These RNA viruses, such as TMV (tobacco mosaic virus), PVX (potato virus X), and TRV (tobacco rattle virus), can been used to silence many different target genes (Angell, et al., (1999) Plant J. 20:357-62; Kumagai, et al., (1995) PNAS 92:1679-83; MacFarlane, et al., (2000) Virology 267:29-35). Other suitable viruses for VIGs construction include, but are not limited to Citrus tristeza virus (CTV), apple latent spherical virus (ALSV), Barley stripe mosaic virus (BSMV), Satellite tobacco mosaic virus (STMV) and Anthoxanthum latent blanching virus (ALBV). Though DNA viruses, limited to Geminiviridae, have not been extensively used as expression vector, tomato golden mosaic virus (TGMV) and cabbage leaf curl virus (CaLCuV) have been used to generate silencing vectors and silenced both transgenes and native genes in tomato and Arabidopsis (Peele, et al., (2001) Plant J. 27:357-66; Turnage, et al., (2001) Plant J. 107:14). As is known to those skilled in the art, each virus/host combination should be optimized for producing effective silencing vectors. In the Examples provided herewith, the viral genome is provided as a bipartite virus. However, it is to be understood that other optimized vectors can be used and are included within the scope of the applicant's teachings. For example, the silencing vector may include the origin of to replication, the genes necessary for replication in a plant cell, and one or more nucleotide sequences with similarity to one or more target genes. The vector may also include those genes necessary for viral movement. In the case of bipartite viruses, for example geminiviruses, the A and B components may be carried in the same silencing vector. Alternatively, as in the case of TRV, the plant may be transformed with both components on separate vectors. In one example, the A genome component of a geminivirus (which replicates autonomously) was shown to be sufficient for VIGS, as was the B component (WO 01/94694 and US Patent Application Publication Number 2002/0148005, both of which are incorporated herein by reference). These references indicate that the A genome (AL1, AL2 and/or AL3) or the B genome (BR1 and/or BL1) may be used as a silencing vector. Other silencing vectors are disclosed in U.S. Pat. No. 6,759,571 and US Patent Application Publication Numbers 2004/0019930 and 20110016584, both of which are herein incorporated by reference. WO 01/94694 (incorporated herein by reference) discloses the locations of the geminivirus genome where the nucleotide sequences may be inserted. For example, the nucleotide sequence that is similar to at least a fragment of a target gene may replace any coding or non-coding region that is nonessential for the present purposes of gene silencing, may be inserted into the vector outside the viral sequences, or may be inserted just downstream of an endogenous viral gene, such that the viral gene and the nucleotide sequence are cotranscribed. For example, the nucleotide sequence may be inserted in the common region of the viral genome, however it is preferred that the nucleotide sequences not be inserted into or replace the Ori sequences or the flanking sequences that are required for viral DNA replication. The size of the nucleotide sequence that is similar to the target gene may depend on the site of insertion or replacement within the viral genome.

Thus, in some embodiments, the nucleic acid agent comprising a nucleic acid which specifically reduces the expression of at least one plant pathogen resistance gene product is comprised in a VIGS viral-induced gene silencing vector construct. In some embodiments, the VIGS vector construct is further comprised in a suitable bacterial host, for example, an Agrobacterium. In yet further embodiments, administering or providing the nucleic acid agent of the invention to the plant comprises introducing a VIGS vector comprising the nucleic acid agent into the cells of a host plant.

Expression comprises transcription of the heterologous DNA sequence into mRNA. Regulatory elements ensuring expression in eukaryotes are well known to those skilled in the art. In the case of eukaryotic cells, they comprise polyA signals ensuring termination of transcription and stabilization of the transcript. The polyA signals commonly used include that of the 35S RNA from CaMV and that of the nos gene from Agrobacterium. Other regulatory elements can include transcriptional and/or translational; enhancers, introns, and others as is known to those skilled in the art.

Any methods of inoculation or transformation may be used as is known to those skilled in the art. The delivery methods for VIGS constructs include but are not limited to, mechanical injection of in vitro transcribed RNA or extracts from infected plants, Agrobacterium (Agro)-inoculation, inoculation by gentle abrasion of the surfaces of the leaves with carborundum and plasmid DNA (“plasmid inoculation”), and microprojectile bombardment. Mechanical injection is time consuming but can increase the efficiency of silencing in certain hosts such as Arabidopsis (Ratcliff, et al., (2001) Plant J. 25:237-45). Agro-inoculation is the most popular and has been developed for both DNA and RNA viruses (Schob, et al., (1997) Mol. Gen. Genet. 256:581-85). Agro-inoculation is more feasible for large-scale production application and less time consuming. Tobacco, tomato, and barley VIGS vectors have been developed and shown extensive silencing using Agro-inoculation. Specifically, TRV-derived VIGS vector/Agro-inoculation is becoming the dominant combination for many investigators. Inoculation by gentle abrasion of the surfaces of the leaves with carborundum and plasmid DNA is described in Uhde, et al., (2005) Arch. Virol. 150:327-340. Microprojectile bombardment of plasmid DNA-coated tungsten or gold micron-sized particles has been extremely useful for DNA virus-based VIGS vector (Muangsan, et al., (2004) Plant J. 38:1004-14). Ryu, et al., (WO 2005/103267) describes a method of VIGS via agroinoculation by drenching roots of the plants in a suspension of Agrobacterium (Agrodrench).

Thus, according to some embodiments of the invention, there is provided a plant comprising at least one exogenous isolated nucleic acid agent comprising a nucleic acid which specifically reduces the expression of at least one plant pathogen resistance gene product selected from the group consisting of plant gene products having at least 60% identity to any of the polynucleotide sequences of Table II or Table III. The plant can be any one of a tree, a shrub, a bush, a seedling, a seed, a scion, rootstock, an inarched plant, a bud, a budwood, a root or a graft. In some particular embodiments the plant is a citrus or citrus-like plant selected from the group consisting of including sweet oranges commercial varieties (Citrus sinensis Osbeck (L.), clementines (C. reticulata), limes (C. aurantifolia), lemon (C. limon), sour orange (C. aurantium), hybrids and relatives (Citranges, Citrumelos, Citrandarins), Balsamocitrus dawei, C. maxima, C. jambhiri, Clausena indica, C. lansium, Triphasia trifolia, Swinglea glutinosa, Micromellum tephrocarpa, Merope spp., Eremolemon; Atalantia spp., Severinia buxifolia; Microcitrus spp., Fortunella spp., Calodendrum capense, Murraya spp., Poncirus trifoliate. In some embodiments, wherein the plant is a citrus or citrus-related plant, the at least one exogenous isolated nucleic acid agent comprises a nucleic acid which specifically reduces the expression of at least one plant pathogen resistance gene product selected from the group consisting of the polynucleotide sequences of Table IV. In some embodiments there is provided a cell of the plant comprising the at least one exogenous isolated nucleic acid agent. The cell can be a cell of any organ or tissue of the plant.

It will be appreciated that some non-citrus plants can also be hosts to Candidatus Liberibacter spp, such as the Solanacaea, for example, tomatoes and potatoes. Tomato has been known to contract C. Liberibacter infection both in the wild and under controlled, laboratory conditions. A non-limiting list of tomato sequences suitable for use with the compositions and methods of the present invention is provided in Table VI:

TABLE VI
SOME TOMATO PLANT PATHOGEN
RESPONSE GENE SEQUENCES
[SEQ ID NO. and GENE IDENTIFICATION (gi) NO.]

| | |
|---|---|
| 489 | 460374150 |
| 490 | 460395563 |
| 491 | 460365404 |
| 492 | 460365402 |
| 493 | 460381148 |
| 494 | 460366991 |
| 495 | 460397655 |
| 496 | 460408956 |
| 497 | 460395273 |
| 498 | 460407453 |
| 499 | 460389683 |
| 500 | 460388052 |
| 501 | 460404051 |
| 502 | 460368826 |
| 503 | 460414034 |
| 504 | 460368828 |
| 505 | 460383569 |
| 506 | 460388054 |
| 507 | 350535071 |
| 508 | 460377611 |
| 509 | 460397175 |
| 510 | 225321497 |
| 511 | 460388987 |
| 512 | 225319432 |
| 513 | 460397173 |
| 514 | 225321666 |
| 515 | 1654139 |
| 516 | 460379599 |

The isolated nucleic acid can be provided in an agrochemical composition. Thus, according to some embodiments, there is provided an agrochemical composition comprising an isolated nucleic acid sequence which specifically reduces the expression of at least one plant pathogen resistance gene product in a plant. As used herein, the phrase “agrochemical composition” is defined as a composition for agrochemical use, and, as further defined, the agrochemical composition comprises at least one agrochemically active substance. Thus, in addition to the isolated nucleic acid sequence, the agrochemical composition of the present invention can include additional plant-beneficial or agrochemically active compounds. Exemplary plant-beneficial or agrochemically active compounds include, but not are limited to fertilizers, antibiotics, biocides, pesticides, pest repellents, herbicides, plant hormones, bacteriocides such as copper and the like. In some particular embodiments, the agrochemical composition comprises plant hormones. As used herein, the term “plant hormone” is used to indicate a plant-generated signaling molecule that normally affects at least one aspect of plant development, including but not limited to, growth, seed development, flowering and root growth. One of skill in the art will readily understand the term plant hormone and what entities fall under the scope of this term. For example, plant hormones include but are not limited to, abscisic acid (ABA) or a derivative thereof, gibberellins (GA), auxins (IAA), ethylene, cytokinins (CK), brassinosteroids (BR), jasmonates (JA), salicylic acid (SA), strigolactones (SL). In select embodiments, the fusion proteins of the present invention comprise a plant hormone binding domain that binds abscisic acid (ABA), gibberellins (GA), auxins (IAA) and/or jasmonates (JA).

Further, the agrochemical composition can optionally comprise with one or more additives favoring optimal dispersion, atomization, deposition, leaf wetting, distribution, retardation of degradation by soil organisms and their secretion (for example, by addition of bacteriocides such as copper), retention and/or uptake of the agrochemical composition by the plant. As a non-limiting example such additives are diluents, solvents, adjuvants, surfactants, wetting agents, spreading agents, oils, stickers, thickeners, penetrants, buffering agents, acidifiers, anti-settling agents, anti-freeze agents, photo-protectors, defoaming agents, biocides and/or drift control agents.

The nucleic acid agents, compositions and agrochemical compositions of the present invention are suitable for agrochemical use. “Agrochemical use,” as used herein, not only includes the use of agrochemical compositions as defined above that are suitable and/or intended for use in field grown crops (e.g., agriculture), but also includes the use of agrochemical compositions that are meant for use in greenhouse grown crops (e.g., horticulture/floriculture) or hydroponic culture systems or uses in public or private green spaces (e.g., private gardens, parks, sports fields), for protecting plants or parts of plants, including but not limited to bulbs, tubers, fruits and seeds (e.g., from harmful organisms, diseases or pests), for controlling, preferably promoting or increasing, the growth of plants; and/or for promoting the yield of plants, or the parts of plants that are harvested (e.g., its fruits, flowers, seeds etc.).

“Agrochemical active substance,” as used herein, means any active substance or principle that may be used for agrochemical use, as defined above. Examples of such agrochemical active substances will be clear to the skilled person and may for example include compounds that are active as insecticides (e.g., contact insecticides or systemic insecticides, including insecticides for household use), acaricides, miticides, herbicides (e.g., contact herbicides or systemic herbicides, including herbicides for household use), fungicides (e.g., contact fungicides or systemic fungicides, including fungicides for household use), nematicides (e.g., contact nematicides or systemic nematicides, including nematicides for household use) and other pesticides (for example avicides, molluscicides, piscicides) or biocides (for example, agents for killing bacteria, algae or snails); as well as fertilizers; growth regulators such as plant hormones; micro-nutrients, safeners; pheromones; repellants; baits (e.g., insect baits or snail baits); and/or active principles that are used to modulate (i.e., increase, decrease, inhibit, enhance and/or trigger) gene expression (and/or other biological or biochemical processes) in or by the targeted plant (e.g., the plant to be protected or the plant to be controlled). Agrochemical active substances include chemicals, but also nucleic acids, peptides, polypeptides, proteins (including antigen-binding proteins) and micro-organisms. Examples of such agrochemical active substances will be clear to the skilled person; and for example include, without limitation: Diamides: chlorantraniliprole, cyantraniliprole, flubendiamide, tetronic and tetramic acid derivatives: spirodiclofen, spirotetramat, spiromisifen, modulators of chordotonal organs: pymetrozine, flonicamid; nicotinic acetylcholine receptor agonists: sulfoxaflor, flupyradifurone; spiroxamines, glyphosate, paraquat, metolachlor, acetochlor, mesotrione, 2,4-D,atrazine, glufosinate, sulfosate, fenoxaprop, pendimethalin, picloram, trifluralin, bromoxynil, clodinafop, fluoroxypyr, nicosulfuron, bensulfuron, imazetapyr, dicamba, imidacloprid, thiamethoxam, fipronil, chlorpyrifos, deltamethrin, lambda-cyhalotrin, endosulfan, methamidophos, carbofuran, clothianidin, cypermethrin, abamectin, diflufenican, spinosad, indoxacarb, bifenthrin, tefluthrin, azoxystrobin, thiamethoxam, tebuconazole, mancozeb, cyazofamid, fluazinam, pyraclostrobin, epoxiconazole, chlorothalonil, copper fungicides, trifloxystrobin, prothioconazole, difenoconazole, carbendazim, propiconazole, thiophanate, sulphur, boscalid and other known agrochemicals or any suitable combination(s) thereof. Other suitable agrochemicals will be clear to the skilled person based on the disclosure herein, and may for example be any commercially available agrochemical, and for example include each of the compounds listed in any of the websites of the Herbicide Resistance Action Committee, Fungicide Resistance Action Committee and Insecticide Resistance Action Committee, as well as those listed in Phillips McDougall, AgriService November 2007 V4.0, Products Section—2006 Market, Product Index pp. 10-20. The agrochemical active substances can occur in different forms, including but not limited to, as crystals, as micro-crystals, as nano-crystals, as co-crystals, as a dust, as granules, as a powder, as tablets, as a gel, as a soluble concentrate, as an emulsion, as an emulsifiable concentrate, as a suspension, as a suspension concentrate, as a suspoemulsion, as a dispersion, as a dispersion concentrate, as a microcapsule suspension or as any other form or type of agrochemical formulation clear to those skilled in the art. Agrochemical active substances not only include active substances or principles that are ready to use, but also precursors in an inactive form, which may be activated by outside factors. As a non limiting example, the precursor can be activated by pH changes, caused by plant wounds upon insect damage, by enzymatic action caused by fungal attack, or by temperature changes or changes in humidity.

The agrochemical composition hereof may be in a liquid, semi-solid or solid form and for example be maintained as an aerosol, flowable powder, wettable powder, wettable granule, emulsifiable concentrate, suspension concentrate, microemulsion, capsule suspension, dry microcapsule, tablet or gel or be suspended, dispersed, emulsified or otherwise brought in a suitable liquid medium (such as water or another suitable aqueous, organic or oily medium) for storage or application. Optionally, the composition further comprises one or more further components such as, but not limited to diluents, solvents, adjuvants, surfactants, wetting agents, spreading agents, oils, stickers, thickeners, penetrants, buffering agents, acidifiers, anti-settling agents, anti-freeze agents, photo-protectors, defoaming agents, biocides and/or drift control agents or the like, suitable for use in the composition hereof.

According to some aspects of the present invention there is also provided a method for manufacturing an agrochemical composition, the method comprising (i) selecting at least one, preferably more, nucleic acid sequence which specifically reduces the expression of at least one plant pathogen resistance gene product in a plant (e.g. dsRNA), and (ii) formulating the nucleic acid agent in a compound with additional substance or substances, such as an agrochemical active substance, or a combination of compounds, and optionally (iii) adding further components that may be suitable for such compositions, preferably for agrochemical compositions. In some embodiments, the compound is comprised in a carrier.

The method of the present invention comprises at least one application of a composition hereof to the plant or to plant parts.

If needed, the composition is dissolved, suspended and/or diluted in a suitable solution before use. The application to the plant or plant parts is carried out using any suitable or desired manual or mechanical technique for application of an agrochemical composition, including but not limited to spraying, brushing, dressing, dripping, dipping, coating, spreading, applying as small droplets, a mist or an aerosol. “Increasing yield, growth rate, vigor, biomass, fruit quality or stress tolerance of a plant when infected by a plant pathogen” as used herein, is the protection of the plant against damage or yield decrease, caused by the plant pathogen infection (as defined earlier). Thus, in addition to the action on the plant pathogen response, the composition hereof can have an insecticidal or an antibiotic or insecticidal activity, helping to combat damage, —and as such prevent yield losses—caused by plant pathogenic organisms.

In some embodiments, the dsRNA or compositions of the invention will be provided to the plant by injection.

Exemplary concentrations of dsRNA in the composition include, but are not limited to, 0.01-0.3 ug/ul, 0.01-0.15 ug/ul, 0.04-0.15 ug/ul, 0.1-100 ug/u1; 0.1-50 ug/ul, 0.1-10 ug/ul, 0.1-5 ug/ul, 0.1-1 ug/ul, 0.1-0.5 ug/ul, 0.15-0.5 ug/ul, 0.1-0.3 ug/ul, 0.01-0.1 ug/ul, 0.01-0.05 ug/ul, 0.02-0.04 ug/ul, 0.001-0.02 ug/ul. According to further embodiments, the concentration of dsRNA in the treating solution includes, but is not limited to, 0.01-0.3 ng/ul, 0.01-0.15 ng/ul, 0.04-0.15 ng/ul, 0.1-100 ng/u1; 0.1-50 ng/ul, 0.1-10 ng/ul, 0.1-5 ng/ul, 0.1-1 ng/ul, 0.1-0.5 ng/ul, 0.15-0.5 ng/ul, 0.1-0.3 ng/ul, 0.01-0.1 ng/ul, 0.01-0.05 ng/ul, 0.02-0.04 ng/ul, 0.001-0.02 ng/ul. According to a specific embodiment, the concentration of the dsRNA in the treating solution is 0.1-1 ug/ul. According to some embodiments, the nucleic acid agent is provided in amounts effective to reduce or suppress expression of at least one plant pathogen resistance gene product. As used herein “a suppressive amount” or “an effective amount” refers to an amount of dsRNA which is sufficient to down regulate (reduce expression of) the target gene by at least 20%, 30%, 40%, 50%, or more, say 60%, 70%, 80%, 90% or more even 100%.

According to some embodiments of the present invention, the concentration of dsRNA is provided to the plant in effective amounts, measured in mass/kg plant. Such effective amounts include, but are not limited to, 0.001-0.003 mg/kg, 0.005-0.015 mg/kg, 0.01-0.15 mg/kg, 0.1-100 mg/kg; 0.1-50 mg/kg, 0.1-10 mg/kg, 0.1-5 mg/kg, 0.1-1 mg/kg, 0.1-0.5 mg/kg, 0.15-0.5 mg/kg, 0.1-0.3 mg/kg, 0.01-0.1 mg/kg, 0.01-0.05 mg/kg, 0.02-0.04 mg/kg, 0.001-0.02 mg/kg, 0.001-0.003 g/kg, 0.005-0.015 g/kg, 0.01-0.15 g/kg, 0.1-100 g/kg; 0.1-50 g/kg, 0.1-10 g/kg, 0.1-5 g/kg, 0.1-1 g/kg, 0.1-0.5 g/kg, 0.15-0.5 g/kg, 0.1-0.3 g/kg, 0.01-0.1 g/kg, 0.01-0.05 g/kg, 0.02-0.04 g/kg, 0.001-0.02 g/kg plant. According to a specific embodiment, the effective amount of the dsRNA provided to the plant is 0.0001-10000 mg/kg plant. In another embodiment, the effective amount is 1-1000 mg/kg plant.

Reagents of the present invention can be packed in a kit including the nucleic acid agent (e.g. dsRNA), instructions for introducing the nucleic acid agent, construct or composition into the plants and optionally an agrochemically active agent.

Compositions of some embodiments of the invention may, if desired, be presented in a pack or dispenser device, which may contain one or more dosage forms containing the active ingredient. The pack may, for example, comprise metal or plastic foil, such as a blister pack. The pack or dispenser device may be accompanied by instructions for introduction to the plant.

According to an exemplary embodiment, the nucleic acid agent, or composition and additives are comprised in separate containers.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials and Experimental Procedures

Gene Target Selection

Target plant resistance response genes are selected according to reported microarray and RNAseq experiments, for example, Tables II, IV and IV(a). Genes from different functional categories are targeted, such as: changes in Salicylic acid levels, changes in Jasmonic acid levels, changes in Gibberellic acid levels, changes in Auxin levels, changes in Cytokinin levels, changes in Ethylene levels, changes in ABA phyto-hormones levels, up-regulation of phyto-hormone-related genes, biosynthesis, deposition and degradation of callose, reactive oxygen species production, functional FLS2/BAK1 complex formation, protein-kinase pathway activation, phloem blockage, starch accumulation, starch accumulation in phloem parenchyma cells, polymer sieve formation, changes in carbohydrate metabolism, cambial activity aberrations, sucrose accumulation and carotenoid synthesis. The specific sequence for targeting is selected according to siRNA analysis available on-line, such as http://www(dot)med(dot)nagoya-u(dot)ac(dot)jp/neurogenetics/i_Score/i_score(dot)html. The selected sequences are ordered synthetically and serve as template for in vitro reverse transcription reaction.

For example, genes and sequences such as those in Table III, and homologues thereof, are selected for targeting and dsRNA targeting them is generated as described below.

Bioassay

Tomato plants are susceptible to C. Liberibacter psyllaurous infection via the psyllid B. cockerelli. Thus, tomato plants constitute a model species for assessing the efficacy of compositions and methods for enhancing fitness of HLB-infected plants.

dsRNA, generated as described above, comprising sequences of selected target tomato genes associated with plant pathogen resistance response is introduced into uninfected tomato plants by dusting, spraying, irrigation, injection or other effective means of delivering the dsRNA to the plant. Exemplary target genes from tomato are detailed in Table VI.

Presence of the dsRNA in the tomato plant tissues and organs is monitored by PCR, gel electrophoresis dot blotting or other typical detection technique, and effective means of delivery are selected. Persistence and integrity of the dsRNA is monitored periodically.

RNA extractions and cDNA syntheses are performed. The cDNA from each replicate treatment is then used to assess the amount and integrity of RNAi by measuring levels of gene expression using qRT-PCR. Reactions are performed in triplicate and compared to an internal reference to compare levels of RNAi. Tomato plants with decreased levels of a tested gene are further grown and experimentally infected with HLB.

Both control and dsRNA-bearing tomato plants are infected with C. Liberibacter psyllaurous (also known as “Lso”) by contacting with infected psyllid or grafting of infected shoots onto tomato rootstock. C. Liberibacter infection is identified by visual inspection for characteristic yellowing, mottling, spotting, leaf curling, stiffness, springiness, purpling, stunting, growth, fruiting etc. and verified by PCR for C. Liberibacter-specific markers.

Fitness of the control and treated tomato plants is assayed according to fruit quality, fruit drop and visual inspection. dsRNA, expression of targeted genes and severity of C. Liberibacter infection are also monitored, in order to assess the correlation between the extent of downregulation of the targeted gene expression, the severity of the disease and the fitness of the treated plants, compared with untreated or sham treated controls.

The present inventors contemplate that introducing dsRNA targeted to tomato plant pathogen resistance response genes into the tomato plants will enhance their fitness and fruit quality following C. Liberibacter (e.g. Lso) infection by attenuating the severity of plant response to infection and preventing or alleviating other adverse effects of plant pathogen resistance response on the infected plant.

Candidate pathogen resistance response genes whose downregulation proves effective in enhancing fitness and fruit quality can serve as valid targets for dsRNA silencing studies in other HLB-susceptible plant species, such as Citrus.

### Example 1

### Screening for Tomato Cultivar Compatibility with Psyllids Yellows (Lso) Disease and with Tobacco Rattle Virus (TRV)-Mediated Virus-Induced Gene Silencing

In order to select tomato cultivars suitable for modeling HBL disease, and the attenuation thereof by gene silencing, expression of the phytoene desaturase (PDS) gene was targeted by viral-induced gene silencing (VIGS), using a tomato rattle virus (TRV) vector and Agrobacterium transformation. Plants exhibiting photobleaching and strong, uniform gene silencing were selected as candidates for infection via psyllid rearing.

Cultivars

Tomato cultivars having a variety of different characteristics were chosen, including open-pollinating-early maturation cultivars (Gold Nugget, Yellow Pear, Early Cascade), open pollination-late maturation cultivars (Manitoba, Prudens Purple, Red Zebra), hybrid-early maturation cultivars (Juliet, Tiny Tim) and Hybrid-Late Maturation cultivars (Big Beef and Celebrity).

Gene silencing Seeds were germinated in water-saturated germination soil mixture in germination cones, cones covered to exclude light and incubated for 48-72 hours at 23-26 degrees C., then transferred to 16/8 light/dark cycle. Seedlings appeared typically after 5 days. The seedlings were then grown to the four true leaf stage (approximately 3 weeks post germination).

Agrobacterium containing constructs pTRV1, pTRV2-Empty Vector (no PDS sequence) and pTRV2-PDS were grown in LB medium+antibiotics overnight, pelleted and resuspended in inoculation medium (10 mM MES, 10 mM MgCl, 250 uM acetosyringone), and OD₆₀₀ measured. pTRV2-PDS contains the tomato phytoene desaturase sequence SEQ ID NO: 520. SEQ ID NO: 519 is the complete tomato PDS sequence. Agrobacterium cultures were mixed 1:1 just prior to infiltration of the plants by spray inoculation. TRV is a bipartite virus and, as such, two different A. tumefaciens strains are used for VIGS—one carrying pTRV1, which encodes the replication and movement viral functions while the other, pTRV2, harbors the coat protein and the plant endogenous sequence used for VIGS. Inoculation of the tomato seedlings with a mixture of both strains results in gene silencing.

Seedlings grown to four-true-leaf stage were inoculated with the transformed Agrobacterium, and grown for another 20 days, and observed for appearance of photo-bleaching (indicating PDS silencing). In order to monitor the levels of target mRNA in the inoculated plants, quantitative PCR analysis was performed on the RNA extracted from homogenized plant material samples, following synthesis of cDNA copies using reverse transcriptase.

Infection of Plants with Lso Via Psyllids

In order to expose the plants to the Lso bacterial pathogen, lower leaves of test plants are covered with an organza bag, one end of which is closed on the leaf, psyllids introduced into the bag (e.g. 15 adult psyllids per treatment) from the other opening and the bag drawn closed to prevent psyllid migration. Test and matching control plants (no psyllids) were returned to normal photoperiod for 72 hours, in order to allow psyllids to feed on the leaves. At 72 hours, the treated leaf (and corresponding control plant leaves) was removed and bag discarded.

Nucleic Acid Extraction

DNA was extracted using cetyl trimethyl ammonium bromide (CTAB) buffer. Extraction of RNA alone was performed using TRI® reagent extraction with DNase treatment to remove DNA.

Results

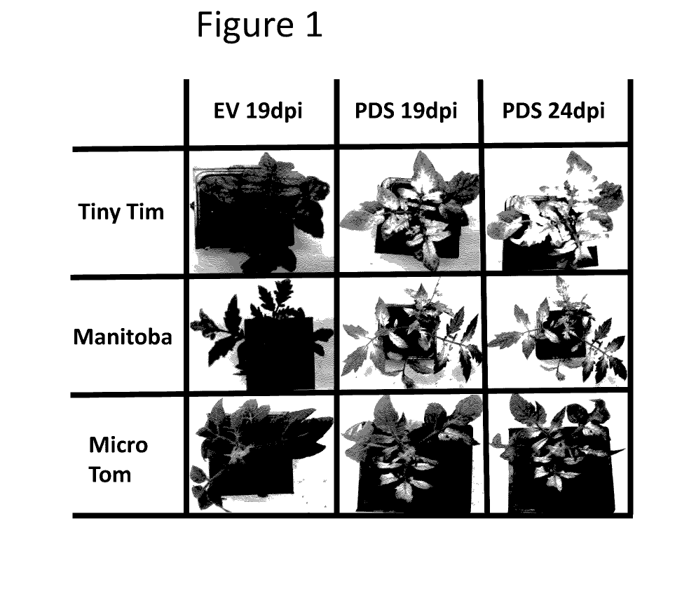

Three cultivars were highly compatible with VIGS silencing (FIG. 1)—Tiny Tim, Microtom and Manitoba. Silencing by infected psyllid rearing in these cultivars resulted in 100% bleaching, indicating 100% silencing rate among tested plants. Moreover, the effect of TRV alone, as judged by the detrimental effect of TRV empty vector (EV), was the least significant.

All the rest of the examined cultivars were deemed incompatible for one of the following reasons: 1. The TRV infection resulted in severe stunting of the plants 2. The photo bleaching appeared at low rates 3. The photo bleaching was very weak and patchy.

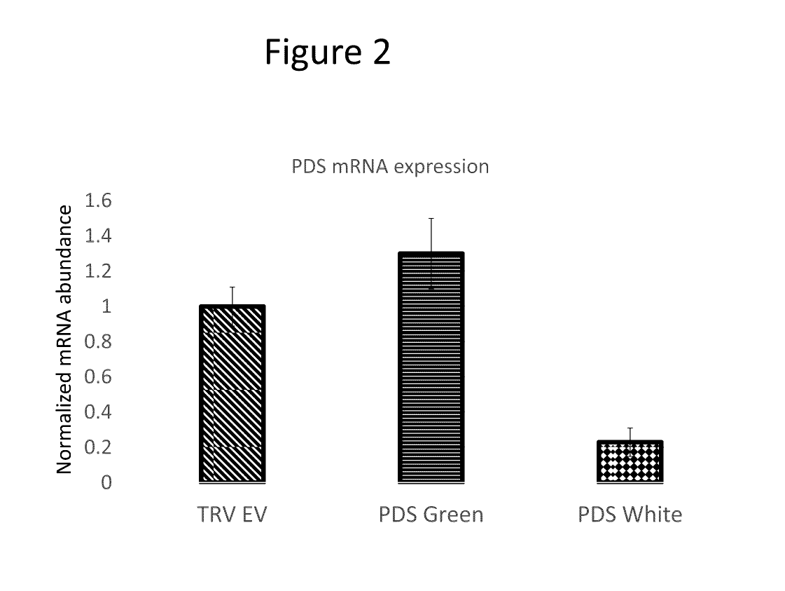

Leaves were picked and processed to determine whether the PDS gene silencing is exclusive to the photo bleaching phenotype. Both green and white leaves were analyzed. The results in FIG. 2 indicate that the white leaf phenotype (PDS white) fully correlated with effective silencing.

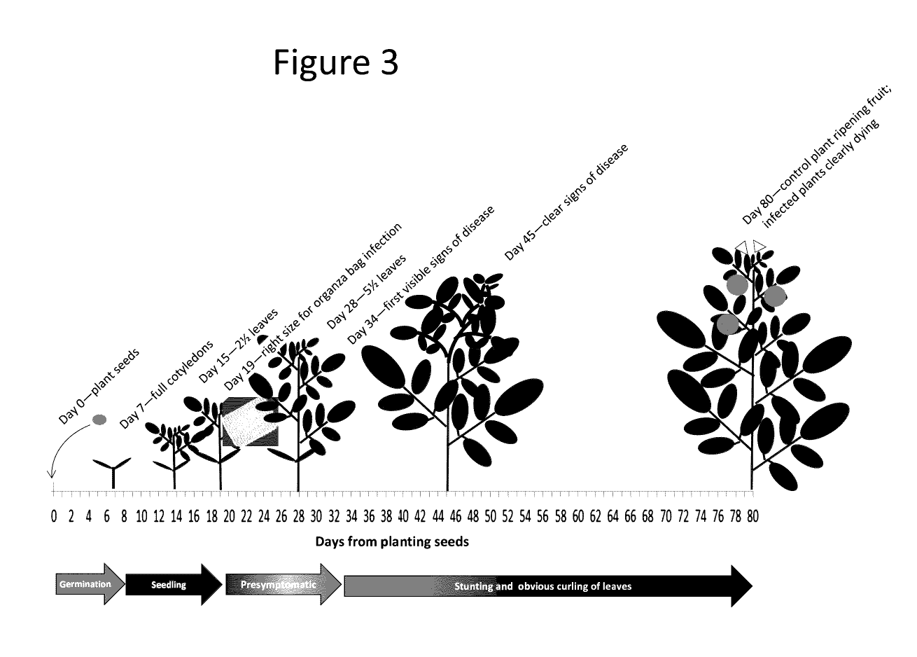

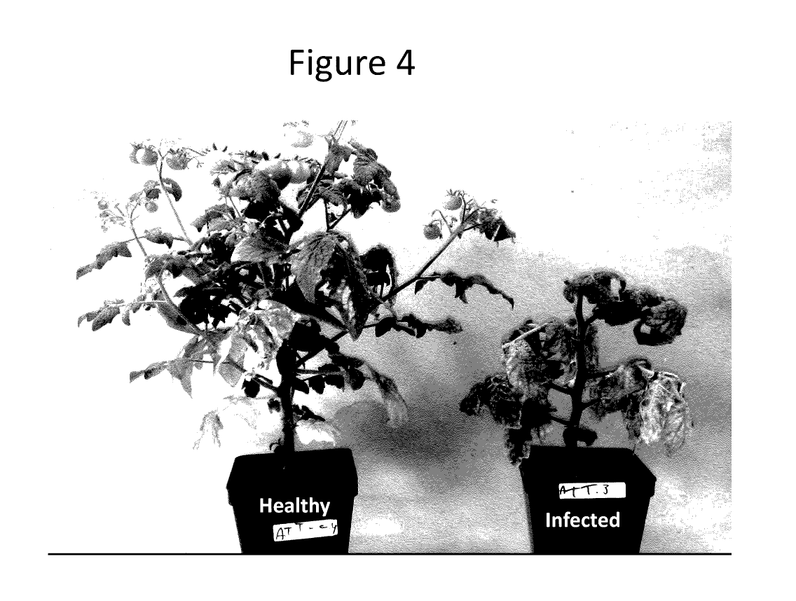

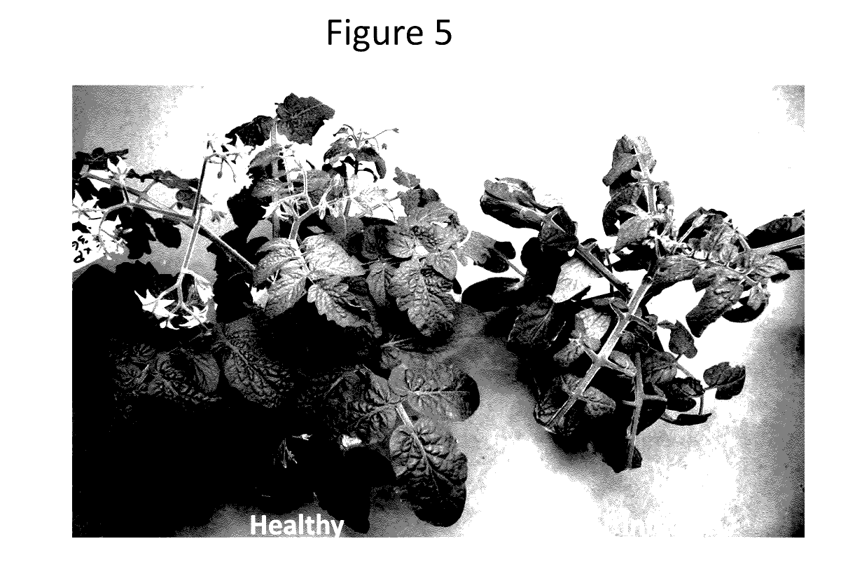

Among the three cultivars that exhibited satisfactory gene silencing, only one was compatible with Lso infection via infected psyllid rearing. Tiny Tim infection resulted in fast progression of disease symptomology. Within 3 weeks post infection, the infected plants were visibly distinguishable from the non-infected plants. FIG. 3 depicts the disease etiology along an 80 day time course. FIGS. 4 and 5 depict the differences between infected and non-infected plants in terms of plant height and number of flowers, respectively.

The presence of actual infection of the psyllid reared tomato plants with the Lso bacterial pathogen was confirmed by PCR using either of two nucleic acid extraction protocols, one in which DNA is extracted, and another extracting RNA. Samples from infected and control plants were assayed for the presence of the Lso 16s ribosomal sequence (SEQ ID NO: 521), using the following primers:

OA2 (also known as CP_P97)

| | |
|---|---|
| GCGCTTATTTTTTAATAGGAGCGGCA | (SEQ ID NO: 522) |

O12C (also known as CP_P98)

| | |
|---|---|
| GCCTCGCGACTTCGCAACCCAT. | (SEQ ID NO: 523) |

Internal control of the PCR was provided by measuring the presence of tomato actin sequences. Tomato actin primers used were:

ACTIN-LIKE7_F Actin2 qRT F (Also known as CP_P23):

| | |
|---|---|
| TTGCTGACCGTATGAGCAAG | (SEQ ID NO: 525) |

ACTINLIKE7_R Actin2 qRT R (Also known as CP_P24)

| | |
|---|---|
| GGACAATGGATGGACCAGAC | (SEQ ID NO: 526) |
| Tomato actin amplicon | (SEQ ID NO: 524) |
| is 291 bases in length. | |

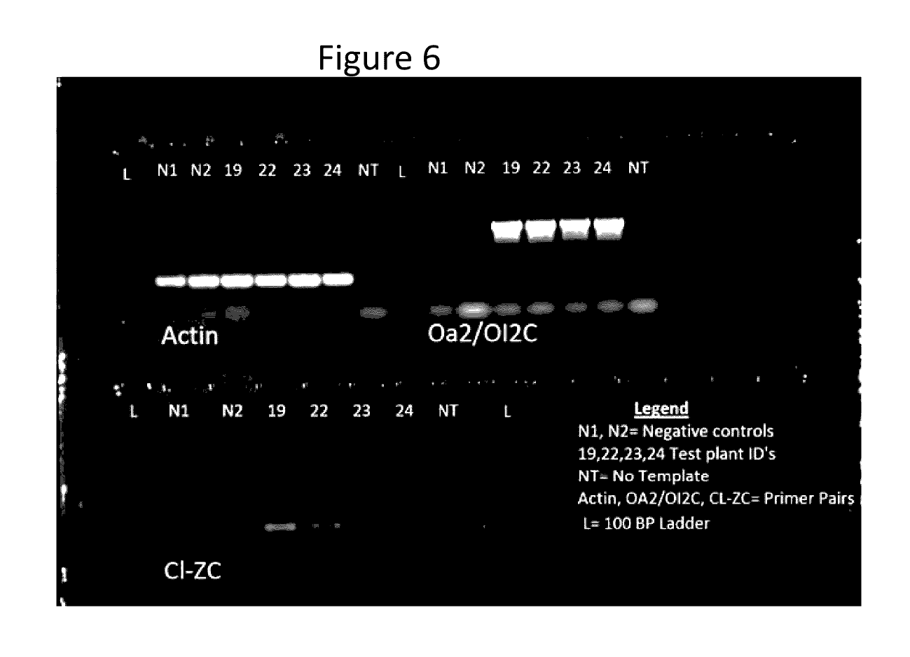

Plant tissue harvested from plants infected via psyllid rearing exhibited clear disease signs at 6 weeks post infection. The non-infected plants (‘mock’ treatment in which an empty ‘organza bag’ was applied similarly followed by snipping of the petiole) showed no signs of the disease (FIG. 6, lanes N1 and N2). The presence of the PCR product that corresponds to the expected size of Lso 16S was detected only in plants that were infected by psyllid rearing (FIG. 6, lanes 19, 22, 23 and 24).

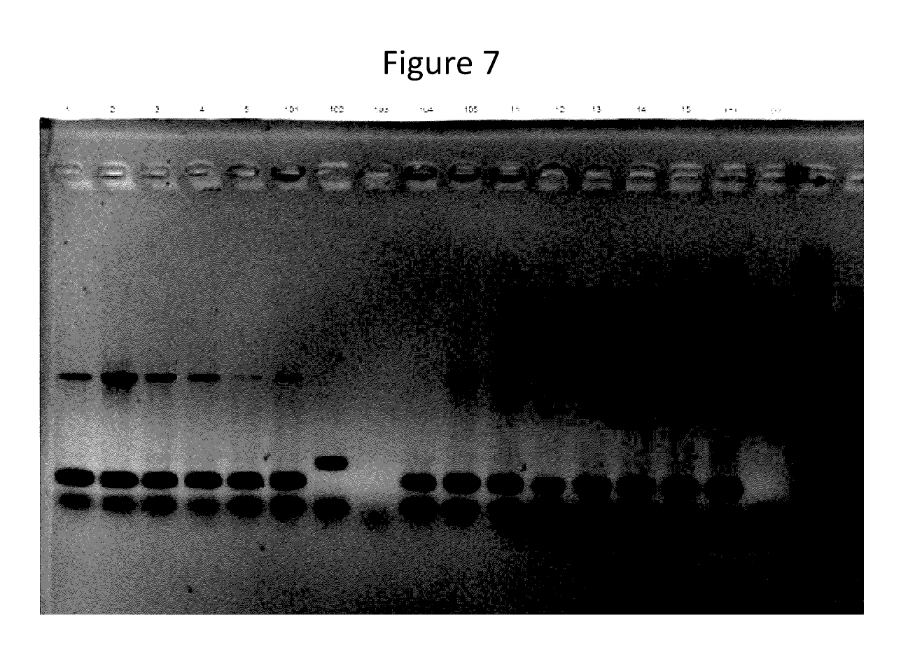

When cDNA was used as template for Lso detection, the detection was in agreement with the severity of disease signs (FIG. 7). The intense bands (Lanes 1, 2, 3, 4, 5 (faint) 101, 105, 11, 12, 14 and 15) corresponded to plants with stronger disease signs. Lower bands are tomato actin (housekeeping gene) controls.

### Example II

### Gene Silencing of Endogenous Tomato Genes Mink Virus-Induced Gene Silencing (VIGS)

Once tomato cultivars suitable for both gene silencing and Lso infection were identified, and infection with the bacterial pathogens confirmed, actual gene silencing of candidate genes was undertaken.

Tiny Tim tomato plants, grown from seed as described above, were agro-infiltrated with Agrobacterium bacterial culture harboring TRV plasmids as described above, using a 1 ml needleless syringe. Targets for gene silencing included genes corresponding to those differentially expressed in HLB infection in citrus:

TABLE VII
Gene Silencing Targets in tomatoes.

| | SEQ ID | | PRIMERS | |
|---|---|---|---|---|
| | | Full | (SEQ ID NOs) | |
| GENE | Target | Sequence | Forward | Reverse |
| AGPase (NCBI | 528 | 527 | 541 | 542 |
| NM_001246989.1) | | | | |
| GPT (NCBI | 530 | 529 | 547 | 548 |
| XM_004240097.2) | | | | |
| Callose Synthase | 532 | 531 | 539 | 540 |
| (NCBI XM_004232827.2) | | | | |
| Lipoxygenase | 534 | 533 | 549 | 550 |
| (GenBank U37840.1) | | | | |
| MYB transcriptional | 536 | 535 | 543 | 544 |
| regulator homologue | | | | |
| (NCBI XM_004249199.2) | | | | |
| PP2-B-15 | 538 | 537 | 545 | 546 |
| (NCBI XM_004230217.1) | | | | |

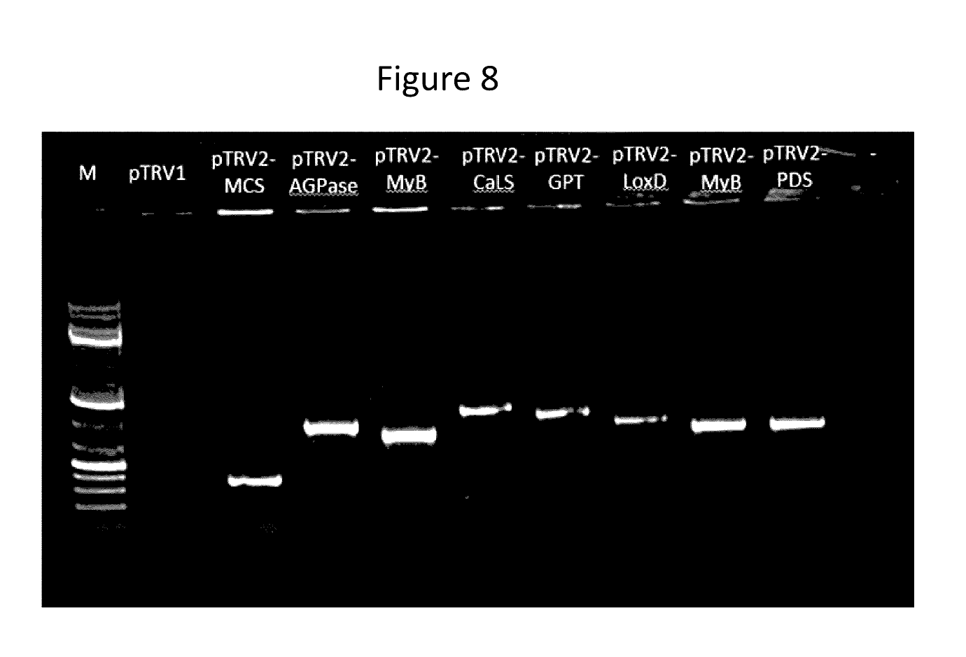

After ligation and transformation into E. coli cells, colony PCR and sequencing were conducted to verify the identity and integrity of each clone (FIG. 8). In FIG. 8, the different sizes of the amplified sequences correspond to the expected lengths of the targets. MCS (multiple cloning site, equivalent to Empty Vector EV) was used as a background control. pTRV1 (plasmid with remaining portion of the TRV genome) produced no amplification.

After agro-infiltration, the plants were kept at 22° C.+/−1 for an additional 22 days before harvesting plant tissue. RNA was extracted and mRNA levels were measured for each gene similarly to the procedure described in Example I.

### Results:

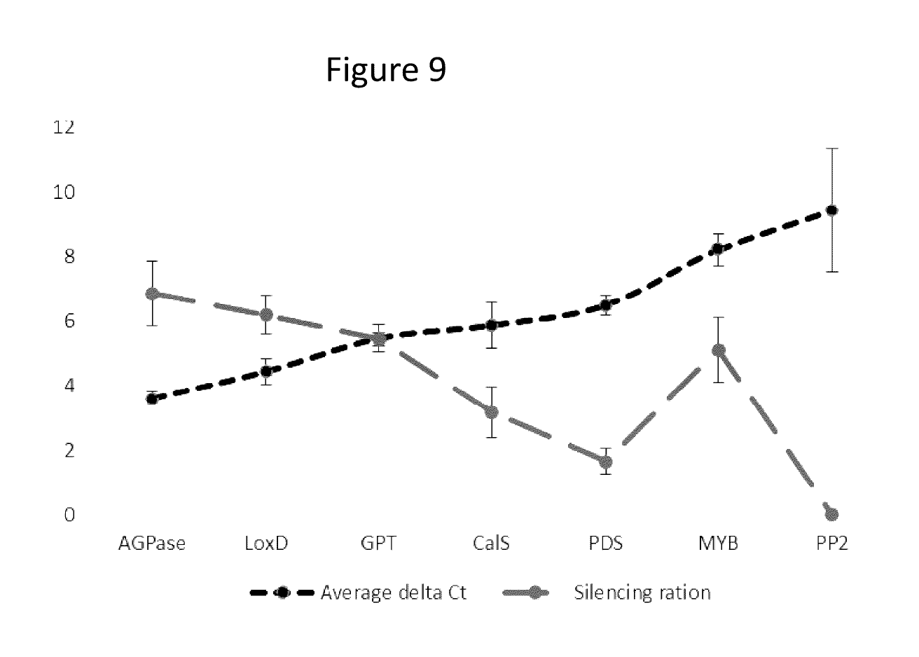

Silencing, to varying degrees, of tomato plant genes is illustrated in FIG. 9. Interestingly, the silencing ratio (the fold decrease of a transcript level in comparison to the empty vector control) was inversely proportional to the basal expression level [the difference (delta) in Ct (cycle threshold—cycle number at which PCR products become detectable) value from the normalizing gene that was used in the qPCR (Actin)] (FIG. 9).

Small RNA Deep Sequencing of Silenced Tomato Plants

Small RNA associated with gene silencing were mapped to identify abundant small RNA species, and map the small RNA distribution along the genes.

RNA was extracted from silenced and control (EV) plants using the MirVana small RNA kit, according to the manufacturer's protocol, and a library representing the population of small RNA fragments (200 bases or fewer) was prepared. The RNA was sequenced, reads smaller than 18 bases were discarded, and siRNA was quantified in the samples either by alignment to the plant genes silencing targets, or by quantification without alignment to the genome and comparison to tomato mature or stem-and-loop miRNA databases.

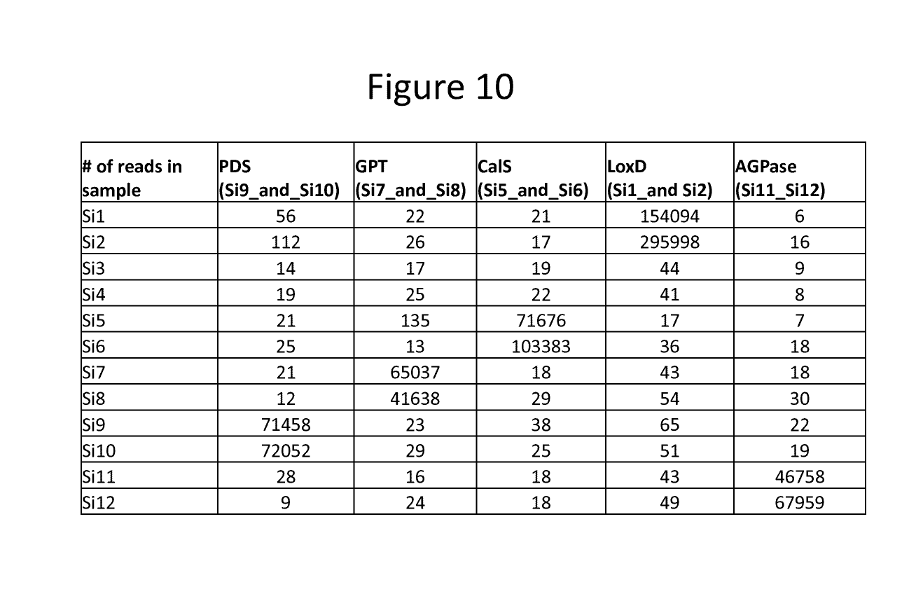

FIG. 10 is a table summarizing mapping of small RNAs against each of the silenced genes, showing that the small RNAs map exclusively to the gene silenced, in each of the silenced genes. Analysis of the read abundance across the sequence of the targeted genes revealed that no significant read abundance was detected outside the “silencing region” for each of the genes assayed.

### Example III

### Differential Gene Expression Resulting from Lso Infection in Tomatoes

The transcriptional response of tomato plants to Lso infection was analyzed, in order to identify potential targets for prevention or mitigation of disease symptomology by gene silencing. Expression profiles were generated at one week prior to developing clear symptoms, i.e. when unequivocal differences start to emerge between the infected and non-infected plants, in an effort to identify genes that are likely to be critical to disease symptomology.

Disease Sign Index

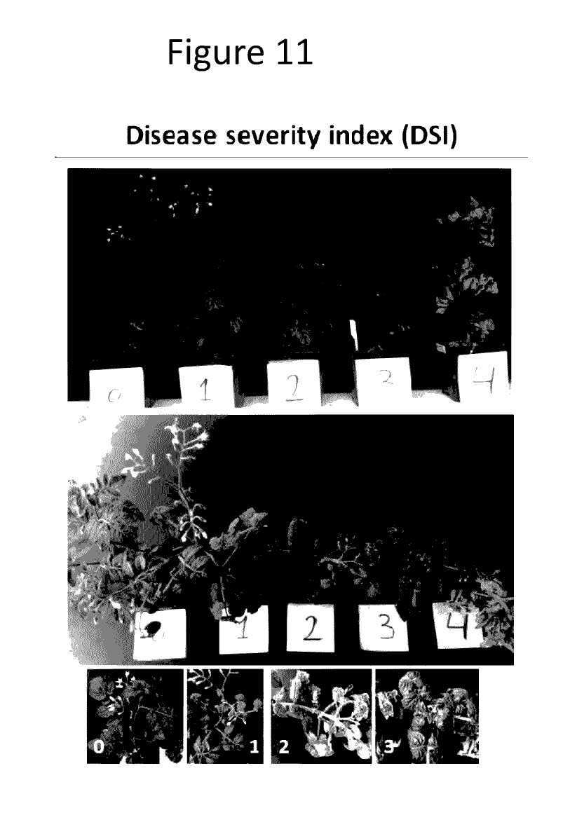

Lso disease severity in tomato plants was assessed visually according to phenotype, by blinded comparison with a standardized disease sign chart (See FIG. 11). The parameters observed include stunting, leaf curling, stiffness and springiness of the leaves, appearance of purple/purplish color, growth and fruiting. “0” DSI is a healthy plant, while a DSI of “4” is considered a severely diseased plant.

In order to prepare an expression profile, 110 Tiny Tim tomato plants were germinated simultaneously. After 15 days, 55 of them were infected via infected psyllid rearing, as described above. The remaining plants were defined as controls, for which a petiole was snipped similarly to the infected plants. At each week post infection, leaf samples were taken in duplicates from 5 infected plants and from 5 control plants, for 8 weeks. Each plant was sampled only once to prevent gene expression changes that result from the sampling itself (injury). All plants were monitored for disease signs according to the DSI (Disease Sign Index) (FIG. 11).

A sample that was taken from a plant that exhibited a DSI level of 1, at one week post sampling was defined as a pre-symptomatic sample. Three such samples were defined, and accordingly three controls plants were paired. RNA was extracted with Trizol (according to above protocol) and verified for high quality by gel electrophoresis and measurement of 230 nm/260 nm and 260 nm/280 nm absorbance ratios (inclusion—at least 2). To verify Lso infection, cDNA prepared from these extractions was used as template for the Lso detection PCR protocol, as described above.

Results

All the three infected plants were positive for Lso infection, while the controls were negative (results not shown). Samples were subjected to microarray analysis using a tomato gene chip (2.0 by Affymetrix). Candidate genes were identified according to the abovementioned criteria, and selected for further investigation as silencing targets.

### Example IV

### Experimental HLB Disease in Citrus Trees

In order to establish a disease model for experiments in a “laboratory”-controlled environment, and in order to synchronize the infections, HLB was introduced into the trees via grafting. The grafted trees can also be used as a template for studying differential gene expression along time.

Grafting Procedure:

Currently, each experiment consists of between 30 to 50 infected plants and an equivalent number of graft controls. The infected trees are roughly six months old and belong to the cultivar ‘sweet orange Valencia’ grafted on top of ‘Swingle’ rootstock.

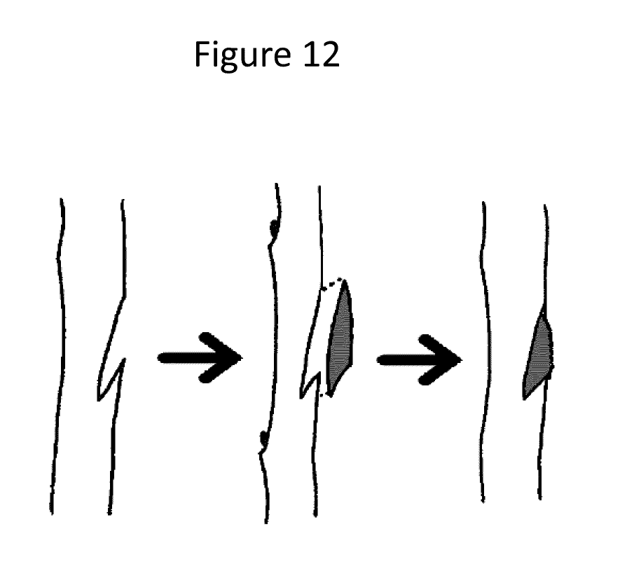

Infection is conducted by grafting two budwoods from different sources onto a tree's stem. The source of grafting material (the budwood) is from infected trees that have been identified as highly symptomatic trees. The ‘graft control’ trees are self-grafted, i.e. a budwood is removed from a tree and grafted again onto the same tree, in order to rule out gene differential expression that results from the injury involved in grafting (FIG. 12).

A modified protocol for the “chip budding graft”, where usually, a bud is taken from one plant and inserted onto another, has been used. As the purpose of the graft here is just to connect vascular tissues and not for vegetative propagation, the scions cut to be used do not need any bud.

Exemplary Grafting Protocol:

1. With a scalpel, make a 45° angled cut to a quarter of the distance through the rootstock, a young non-lignified stem from a healthy Citrus sp. Four centimeters above the first cut, make a second downward and inward cut until it meets the first cut to create a small notch and remove the chunk of bark;

2. With a scalpel, cut a piece of the scion, a stem of the HLB infected Citrus sp., in a root-apical direction, with the same size of the chunk cut of the rootstock. This scion should have the same caliber of the rootstock's stem cut;

3. Insert the stem infected with HLB cut (scion) into the cut of the healthy plant's stem (rootstock), reassuring that both stems are in the same vascular flow direction (root-apical);

4. Wrap the region with a plastic strip as tight as possible;

5. After forty days take the plastic strip off, if the infected stem died, there is a high possibility that the bacteria failed to transmit in the healthy tissue, therefore discard the tree;

6. If the grafting succeeded (the infected stem is still alive), after two months, the grafted plant can be tested (from a leaf sample above the graft) by PCR reaction to evaluate success of infection;

7. Monitor growth effects, symptoms should appear from 4 months;

Control Group:

To produce a control plant, use the same procedure described above, but performing only with healthy plants (no infected scions).

HLB Detection by Conventional PCR.

In order to assess the success rate of huanglongbing (HLB) infection of grafted citrus plants and to confirm that control plants are free of these bacteria, HLB specific DNA can be detected by conventional PCR.

DNA is extracted from citrus similarly to the DNA extraction protocol described above for tomato.

To detect the presence or absence of Ca. L. asiaticus, Ca. L. africanus and Ca. L. americanus in citrus plants, a duplex PCR is performed using bacterial-specific primers, for example, targeting the β—operon ribosomal protein gene of Ca. L. asiaticus and Ca. L. africanus and the 16S rDNA of Ca. L. americanus.

Exemplary Primers for PCT Detection of HLB Infection:

Ca. L. americanus:

(SEQ ID NO: 715)

| | |
|---|---|
| GB1-forward primer: | AAGTCGAGCGAGTACGCAAGTACT |

(SEQ ID NO: 716)

| | |
|---|---|
| GB3-reverse primer: | CCAACTTAATGATGGCAAATATAG |

Ca. L. asiaticus and Ca. L. africanus:

(SEQ ID NO: 717)

| | |
|---|---|
| A2-forward primer: | TATAAGGTTGACCTTTCGAGTTT |

(SEQ ID NO: 718)

| | |
|---|---|
| J5-reverse primer: | ACAAAAGCAGAAATAGCACGAACAA |

Additional Primers:

(SEQ ID NO: 719)

| | |
|---|---|
| CaLas PGK_RT_L3 forward | CAATCGTGGGAGGCTCTAAG |

(SEQ ID NO: 720)

| | |
|---|---|
| CaLas PGK_RT_R3 reverse | CCATGCCCTGTGCTACTAA |

(SEQ ID NO: 589)

| | |
|---|---|
| Citrus 18S forward | GCTTAGGCCAAGGAAGTTTG |

(SEQ ID NO: 590)

| | |
|---|---|
| Citrus 18S reverse | TCTATCCCCATCACGATGAA |

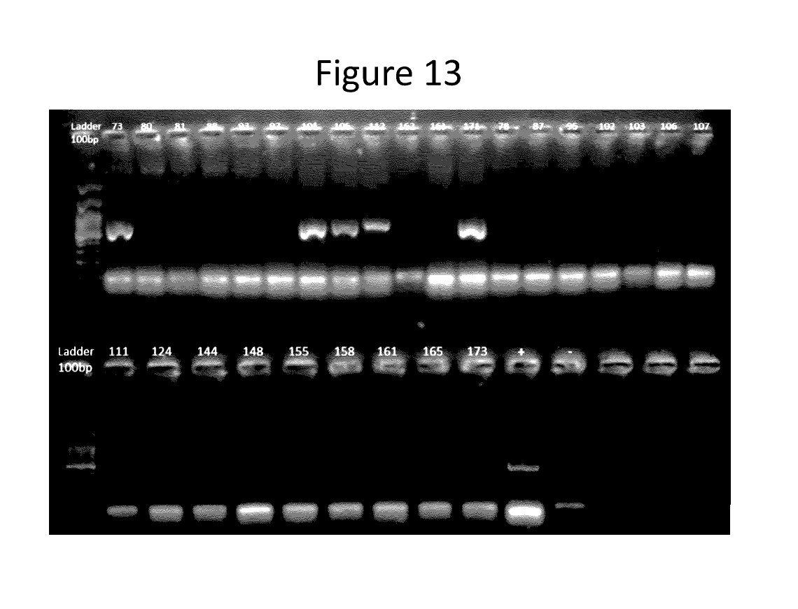

FIG. 13 shows PCR products from samples from infected and control trees, on separated on an agarose gel (two gels are shown). The lower band is the actin amplicon (positive control). The upper band is C. liberibacter 16S, evidence of HLB infection of the sample trees. “+” is a positive control (C. Liberibacter DNA), and “−” is a negative control (uninfected trees). Trees 73, 101, 105, 112 and 171 are clearly positive for HLB infection.

### Example V

### HLB Infection-Sensitive Parameters—Plant Height

One of the most pronounced symptomatic responses of young citrus trees to HLB infection is vertical growth retardation. Establishment of a quantitative measurement of this parameter is important to the ability to assess results of treatment(s). Trees were pruned to 50 cm, and then measured for 8 months. Careful measurement of both infected trees and uninfected control trees over the eight months revealed that height differences emerge approximately at 5 months post infection, and proceed to increase, consistently, in successive months:

TABLE VIII

| Months Post-Infection | Control Height (cm) | Infected Plant Height (cm) |
|---|---|---|
| 0 | 50 | 50 |
| 1 | 53.8 | 53.7 |
| 2 | 57.1 | 58.0 |
| 3 | 59.3 | 58.4 |
| 4 | 61.3 | 60.1 |
| 5 | 63.9 | 61.3 |
| 6 | 69.3 | 62.9 |

### Example VI

### Differential Gene Expression in HLB

Genes that are up-regulated in response to HLB (differentially expressed between infected and non-infected trees), and genes that are up-regulated in response to infection only in a susceptible strain, but not (or to a lesser degree) in a tolerant strain, are attractive targets for silencing. One example is callose synthase, responsible for callose deposition in the phloem. Thus, a callose synthase gene whose regulation adheres to the criteria mentioned above was selected for gene expression analysis. mRNA abundance of genes of interest was compared between infected and non-infected groups of plants, both from experimental groups (e.g. grafting experiments) and from field samples collected from commercial orange groves.

For each measurement, ten infected and ten non-infected samples were sampled. RNA was extracted according to the protocol described above and cDNA was synthesized accordingly. mRNA levels were compared using the SYBR® Green (Life Technologies, Carlsbad Calif.) protocol for detection of PCR products. mRNA levels were calculated using differential Ct (cycle threshold) calculation (see above). The 18S gene was used as normalizing transcript.

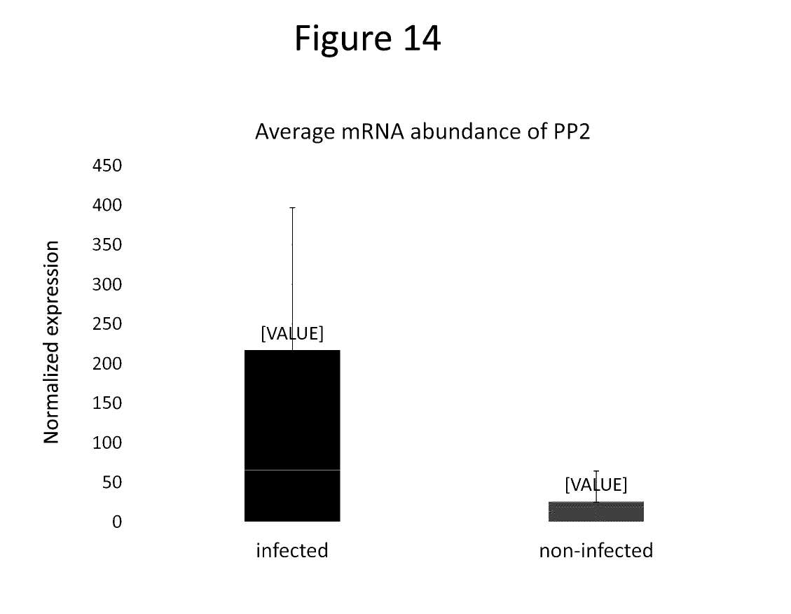

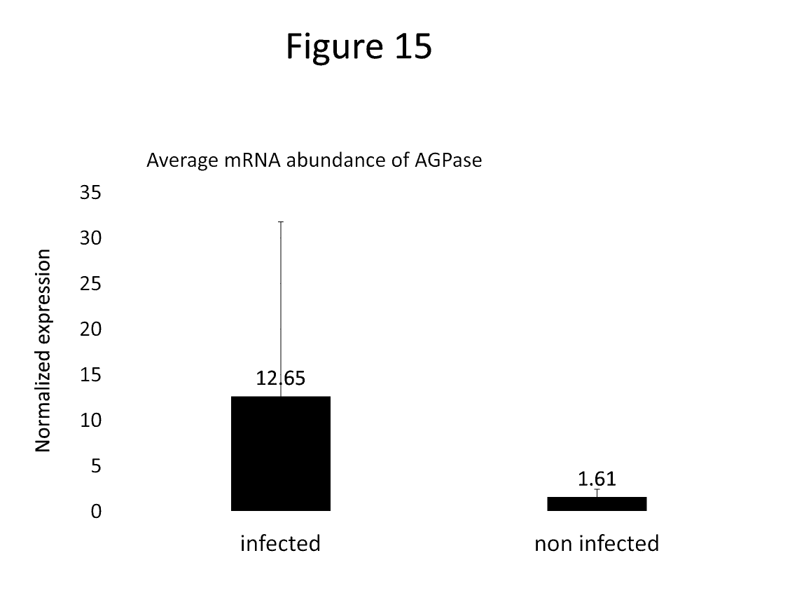

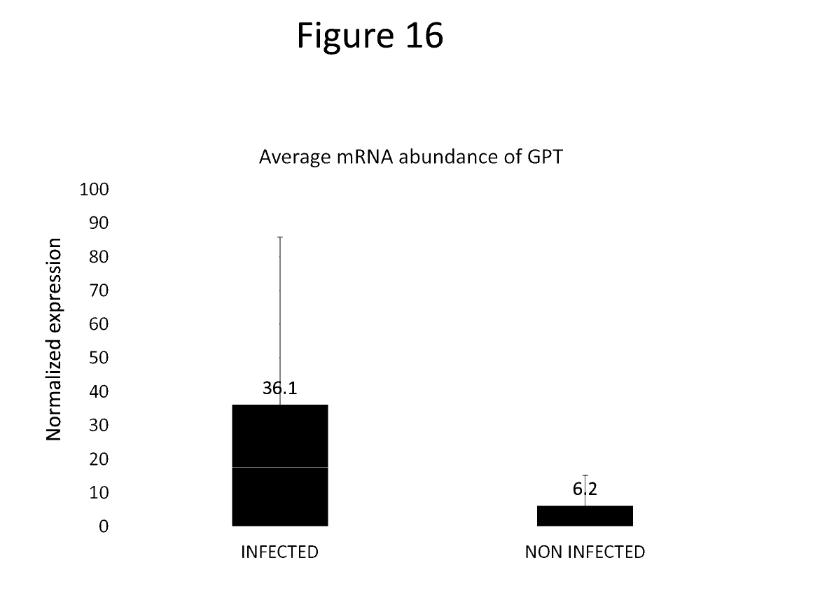

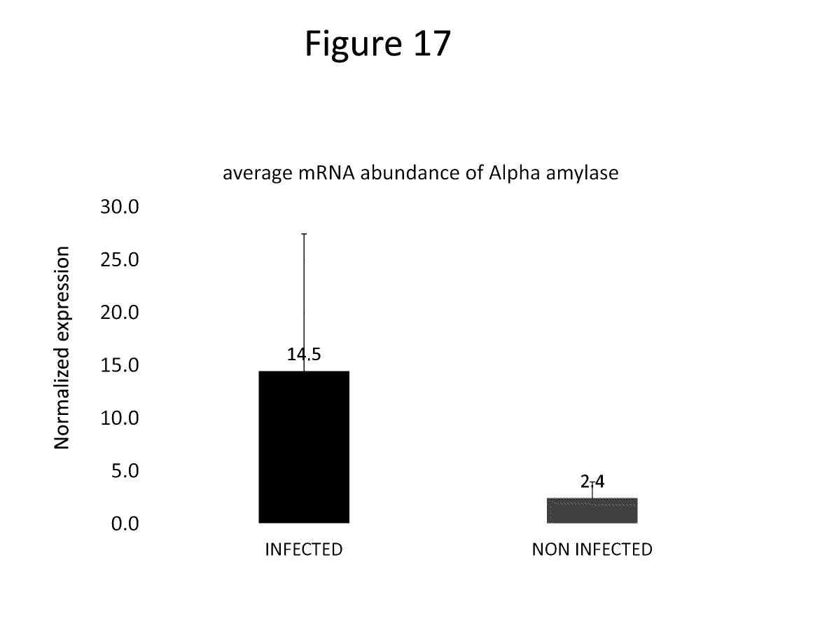

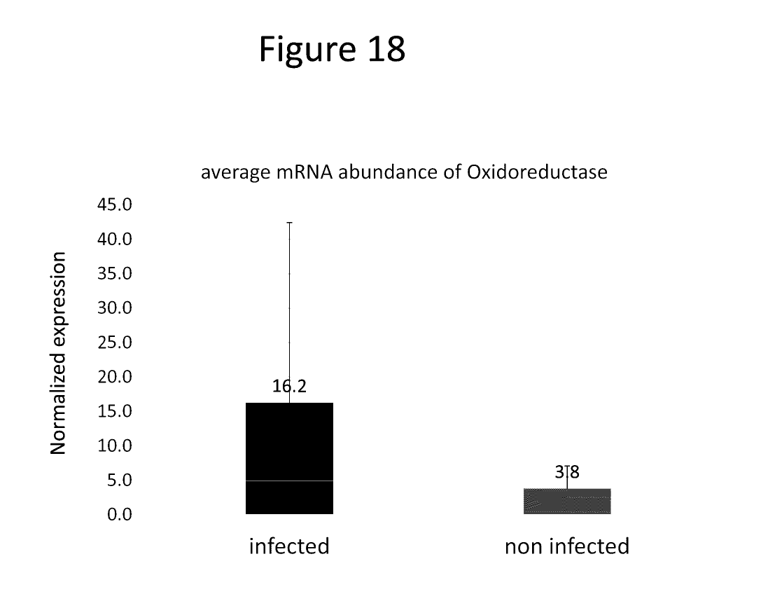

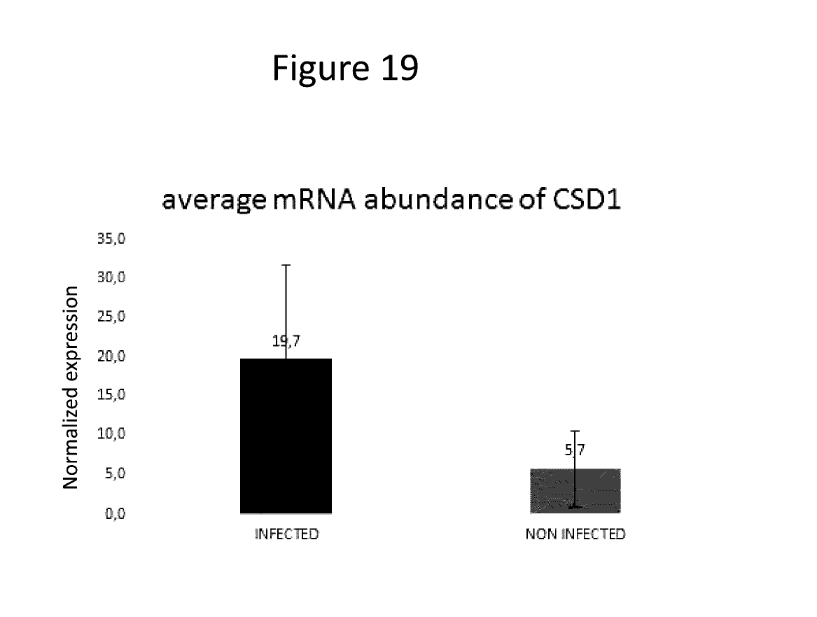

Average relative mRNA abundance levels detected for PP-2 (phloem-specific lectin PP2-like protein) (FIG. 14), AGPase (ADP-Glucose phosphorylase large subunit) (FIG. 15), GPT (Glucose-6-phosphate translocator protein) (FIG. 16), putative alpha-amylase protein (FIG. 17), oxidoreductase (FIG. 18) and cytosolic copper/zinc superoxide dismutase (CDS1) (FIG. 19) indicated a strong upregulation in the infected as compared with uninfected, control trees. Sets of primers that were used for measurements are depicted below.

| Annotation | direc- | Sequence |
|---|---|---|
| (SEQ ID NO) | tion | (SEQ ID NO:) |
| Phloem-specific | F | AGATTAGTGTTGCCGCTGGT |
| lectin PP2-like | | (551) |
| protein NCBI | | |
| XM_006433409.1 | R | GAAGGAAGGGTTTCCAGGTC |
| (571) | | (552) |
| ADP glucose pyro- | F | CATTCGTTCAGGAATCACCA |
| phosphorylase large | | (553) |
| subunit NCBI | R | CTTCTTTCCAGGCCAAAATG |
| XM_006487154.1(572) | | (554) |
| Alpha-amylase, | F | GCTTACATCCTCACACATCCC |
| putative | | (555) |
| XM_006473264.1(574) | R | CCATCTTTGGTCCAATCTTCA |
| | | T(556) |
| Glucose-6-phos- | F | TT GTGTGGTGGGTAGC |
| phate/phosphate | | (557) |
| translocator, | | |
| putative NCBI | R | GAGCATTGACGGGTTGA |
| XM_006467936.1(573) | | (558) |
| citrus 18S rRNA | F | GCTTAGGCCAAGGAAGTTTG |
| | | (559) |
| | R | TCTATCCCCATCACGATGAA |
| | | (560) |
| cytosolic cop- | F | CTGGAACTAACGGTCGCAAG |
| per/zinc superoxide | | (563) |
| dismutase | R | AAGTGTGAATAATGAGTGCGTGA |
| CSD1 GenBank: | | (564) |
| AJ000045.1(577) | | |
| oxidoreductase, | F | ACCAGCTTCCTCGTTTGTGT |
| zinc binding | | (567) |
| dehydrogenase | R | TCAAGGTGGGAAAATGCTTC |
| family protein NCBI | | (568) |
| XM_006470280.1(575) | | |
| Callose synthase | F | TTCCTCTTCAACCCATCAGG |
| | | (569) |
| NCBI XM_006482747.1 | R | TCCAATCTGTCCAGTCATCAA |
| (576) | | (570) |

### Example VII

### Fine Analysis of Differential Gene Expression by Signal Amplification

Signal amplification, as opposed to molecular amplification (as in PCR) of nucleic acid sequences provides a sensitive tool for measuring gene expression along the infection cycle of HLB, since it is not limited to fold-changes as in PCR. The expression dynamics of eight different genes, from three different time points were monitored and compared.

For each of the three time points, ten trees were sampled in triplicate. Five trees were verified for infection by PCR (as described above) and three were graft controls that were verified to be bacteria free. (The exception was time point ‘1 months post infection’ for which it was too soon to verify infection since HLB bacteria could be detected only after 8-20 months post grafting).

Sample preparation for signal amplification (Quantigene 2.0®, Affymetrix, Inc, Santa Clara, Calif.) is typically as follows: 400 uL of homogenization solution with 4 ul proteinase K was added to each sample. Each sample was homogenized at 25 Hz for 15 minutes per cycle, for a total of 3 cycles, incubated at 65° C. for 30 minutes, and centrifuged to pellet debris. Each homogenate was then transferred to a new tube, resedimented to clarify, and aliquoted to the hybridization plate and processed according to the manufacturer's protocol.

The genes analyzed by signal amplification include GPT (NCBI Reference Sequence: XM_006449009.1, SEQ ID NO: 721), Alpha amylase (NCBI Reference Sequence: XM_006473264.1, SEQ ID NO: 722), PP2 (NCBI Reference Sequence: XM_006472910.1, SEQ ID NO: 723), AGPase NCBI Reference Sequence: XM_006423259.1, SEQ ID NO: 724), Zinc transporter (NCBI Reference Sequence: XM_006448556.1, SEQ ID NO: 725), MYB transcriptional regulator (NCBI Reference Sequence: XM_006429779.1, SEQ ID NO: 726), CDR1 (NCBI Reference Sequence: XM_006437293.1, SEQ ID NO: 727), Cu/Zn Superoxide dismutase (GenBank: AJ000045.1, SEQ ID NO: 577), Elongation factor 1 HKG (Gene ID: 102578002, SEQ ID NO: 729) and Actin like-HKG (NCBI Reference Sequence: XM_006492793.1, SEQ ID NO: 730).

Results

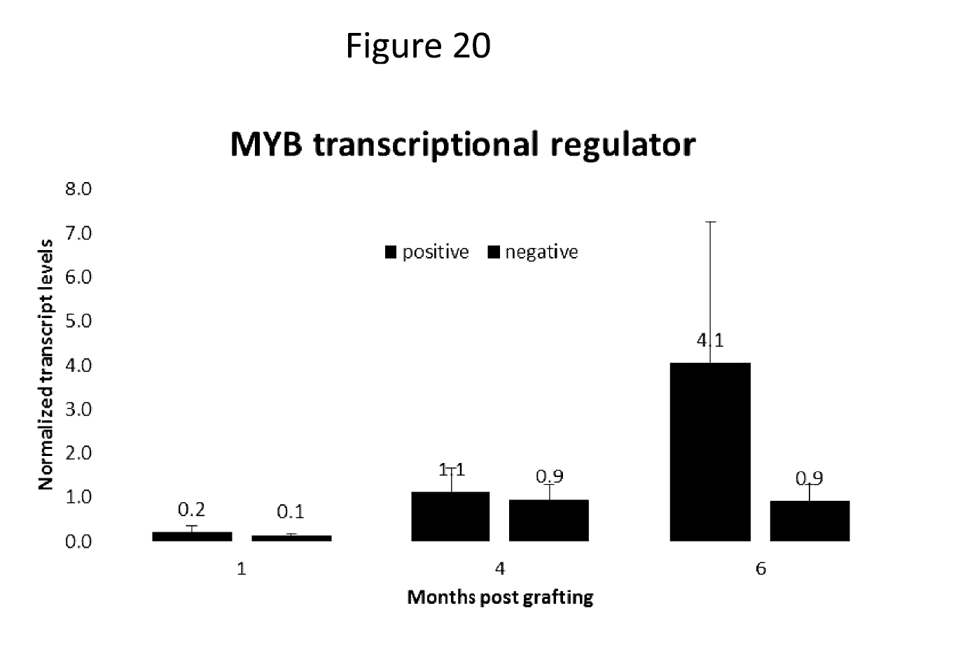

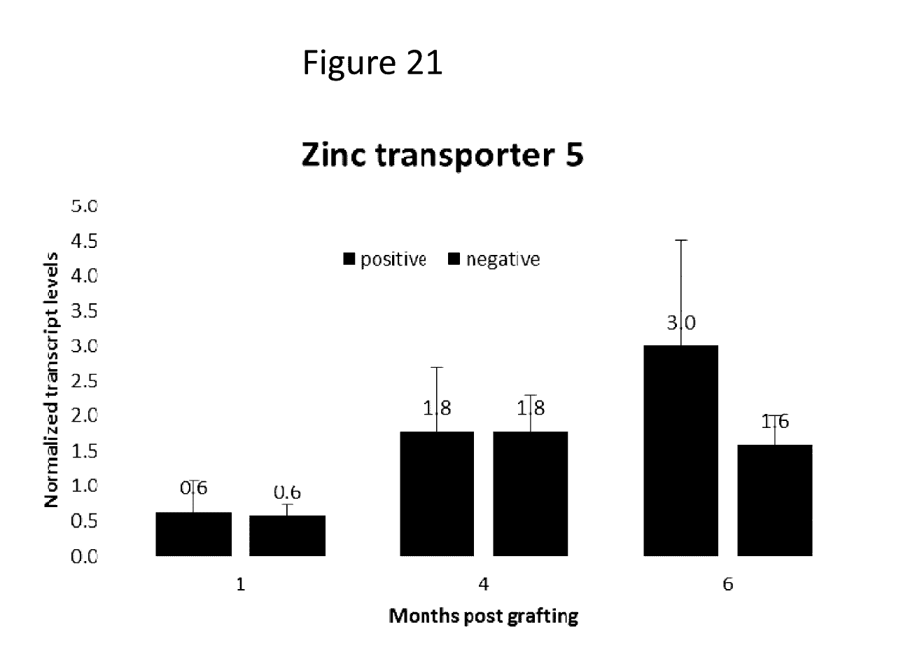

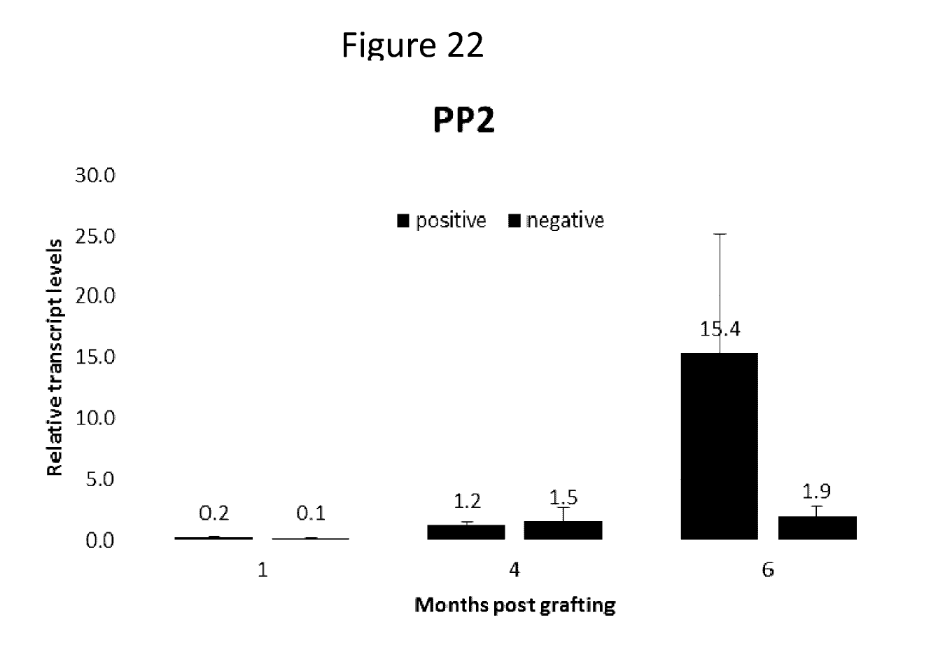

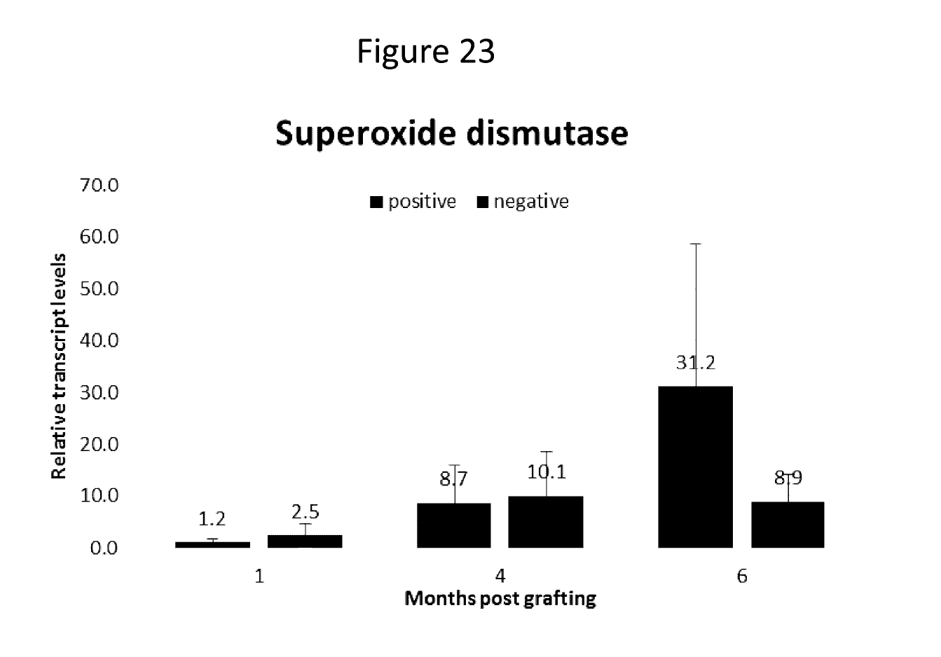

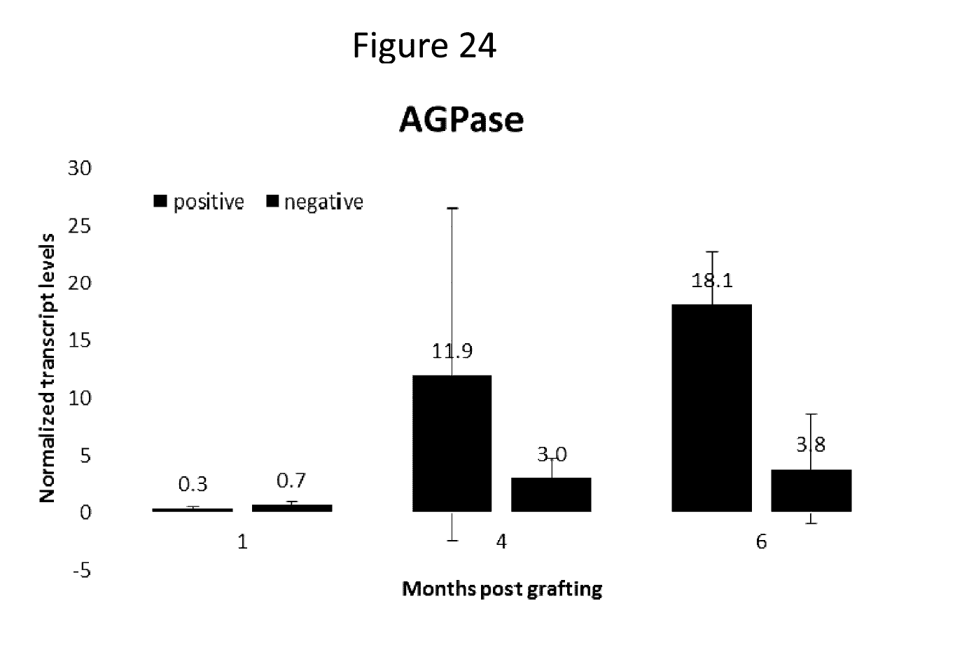

Gene expression was normalized to Actin. Results of gene expression with signal amplification indicated that while certain genes, such as superoxide dismutase (FIG. 23), PP2 (FIG. 22), Zinc transporter (FIG. 21) and MYB (FIG. 20) were differentially expressed only in the later stages of infection (4 and 6 months), other genes (e.g. AGPase, FIG. 24) were differentially expressed at the early stages of the disease, information useful in understanding the transcriptional response to the disease and strategizing regimens for treatment with dsRNA.

### Example VIII

### Analysis of Starch Accumulation Dynamics in HLB Infected and Non-Infected Citrus Plants

Starch is the major and the most abundant storage polysaccharide in plants and is a primary product of photosynthesis deposited transiently in the chloroplast in the form of insoluble granules. It is synthesized inside plastids, but its function depends upon the particular type of plastid and the plant tissue from which it is derived. Starch synthesis can be influenced by day length, night temperature and the time of the day. Normally, all starch synthesized during the light period is degraded during the night, supplying sugars needed for metabolism in the whole plant. In general, starch biosynthesis starts with the formation of ADP-glucose and then transfer of the glucose moiety on to an acceptor, usually a short chain of malto-oligosaccharides and at the end of whole processes in which participate numerous enzymes they build up final structure of starch granule.

A major symptom associated with HLB infection is massive starch build up in leaves and petioles. HLB affected trees have starch accumulated extensively in photosynthetic cells as well as phloem elements and vascular parenchyma in leaves and petioles. In contrast, roots from HLB-affected trees are depleted of starch whereas roots from control trees contain substantial starch deposits. Starch accumulation, however, is also observed in response to nutritional deficiencies and viral infection.

Investigation of the host response through microarray analysis indicated that HLB infection up-regulates starch biosynthetic enzymes: three key starch biosynthetic genes including ADP-glucose pyrophosphorylase, starch synthase, granule-bound starch synthase and starch debranching enzyme likely contributed to accumulation of starch in HLB affected leaves.

Starch Content Assay

Simple measurement of the starch content of plant tissues involved solubilizing the starch, converting it quantitatively to glucose and assaying the glucose. Plant tissue must initially be frozen rapidly to arrest metabolism, then extracted to remove free glucose. Starch is solubilized by heating, then digested to glucose by adding glucan hydrolases. Glucose is assayed enzymatically. Iodine-based protocols can also be used, however, they tend to be less sensitive and less accurate, while the enzymatic assays are more suitable for tissues that have a wide range of starch contents.

Briefly, starch measurement is performed as follows:

Starch Extraction:

Leaf tissue is harvested, flash frozen, ground (e.g. in a mortar and pestle), weighed and extracted with ethanol. Starch is pelleted and washed with ethanol, dried and reconstituted in water. Starch is then gelatinized by autoclaving, then digested with alpha-amylase and amyloglucosidase.

Glucose content of the digested starch samples is measured by the Hexokinase assay [Glucose (HK) Assay, Sigma, St Louis, Mo.]. Glucose is phosphorylated by adenosine triphosphate (ATP) in the reaction catalyzed by hexokinase. Glucose-6-phosphate (G6P) is then oxidized to 6-phosphogluconate in the presence of oxidized nicotinamide adenine dinucleotide (NAD) in a reaction catalyzed by glucose-6-phosphate dehydrogenase (G6PDH). During this oxidation, an equimolar amount of NAD is reduced to NADH. The consequent increase in absorbance at 340 nm is directly proportional to glucose concentration.

### Results:

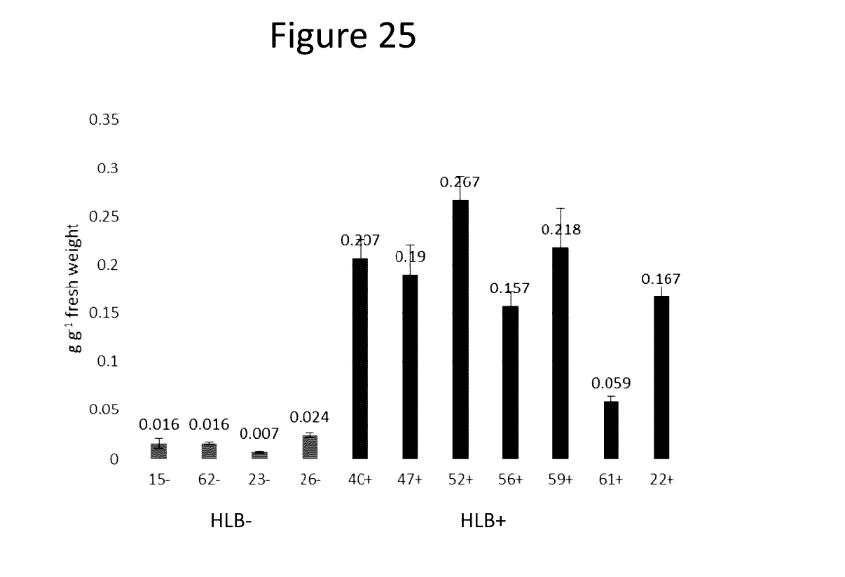

FIG. 25 shows the greatly increased starch content (gram starch per gram fresh weight) of leaves of infected (HLB+) compared with uninfected control leaves (HLB−) of citrus trees at a single time point, six months after infection.

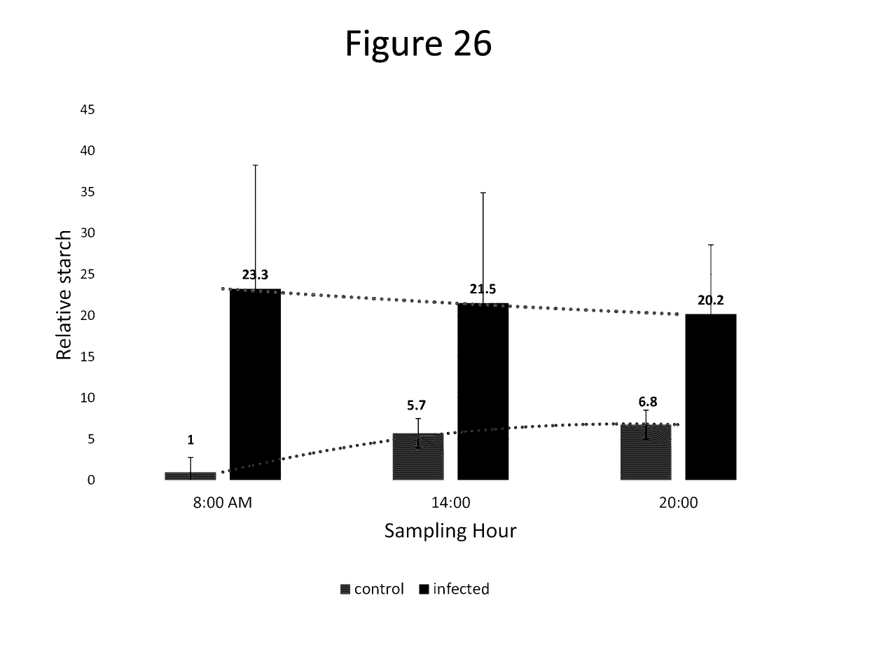

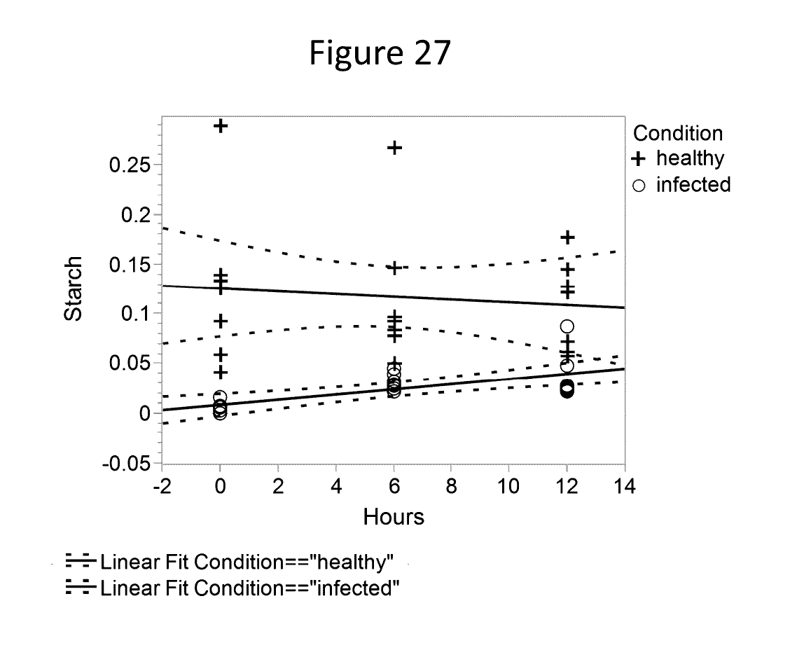

FIG. 26 shows the dynamics of starch accumulation in infected and healthy uninfected control trees. The relative starch content [standardized to starch content of control leaves—i.e. starch content of control leaves at 8:00 AM=1] of leaves from infected and control trees was calculated at 8:00 AM, 14:00 and 20:00. While the control plants accumulated starch during the day (statistically significant positive slope), the much greater starch content of the infected plants showed no statistically significant changes during the day (FIG. 27).

### Example IX

### Lso Disease Sign Mitigation by Gene Silencing

The effect of gene silencing on disease sign in LSO infected tomato plants can be measured quantitatively, for example, according to differences in starch content of the leaves, or semi-quantitatively using phenotypic scoring, and correlated molecular data indicating gene silencing and the presence of LSO.

Plants

Tiny Tim tomato plants were germinated as described above. Plants having two or more true leaves at 6-9 days were excluded from the experiment.

VIGS Infiltration

VIGS infiltration was conducted using a 1 ml needleless syringe at the bottom side of the cotyledon, at 6-9 days post germination, as described above.

290 plants were divided into the following groups:

20 untreated controls

50 empty vector (EV)

40 PDS (photobleaching silencing control)

30 each for GPT silencing, AGPase silencing, PP2 silencing, CalS silencing, LoxD silencing and MYB silencing.

Plants were kept at 22 degrees C. for optimal gene silencing. After 20 days, all PDS plants (100%) displayed substantial photo bleaching, suggesting robust gene silencing for all treatment groups.

After exactly two weeks Lso infection was conducted as previously described, according to the following grouping: 10 plants NO VIGS infected, 10 plants NO VIGS uninfected, 30 plants EV infected, 20 plants EV uninfected, 20 plants PDS infected, 20 plants PDS uninfected, 20 plants each individual gene silencing treatment infected and 10 plants each individual gene silencing treatment uninfected.

Analysis and Sampling:

Plants were observed at each week after the infection for four weeks. Samples were taken from all plants at two and four weeks after infection.

At each week, the DSI (Disease Sign Index) index was measured in a ‘double blind’ procedure.

RNA was extracted from 4 weeks post infection samples, converted to cDNA and submitted to Lso detection protocol, since by that time all infected plants can be detected by PCR. 94% of the plants were confirmed for infection. Plants that were identified as non-infected were excluded from both the phenotypic and molecular (gene expression) analysis.

Results

In an ideal situation, candidate HLB-associated genes are up-regulated with infection. While it has been observed that VIGS suppresses gene expression in naïve plants to a certain degree, the combination of VIGS and bacterial infection restores the basal expression levels such that if the gene's up-regulation is responsible for disease symptoms, those would be mitigated upon treatment.

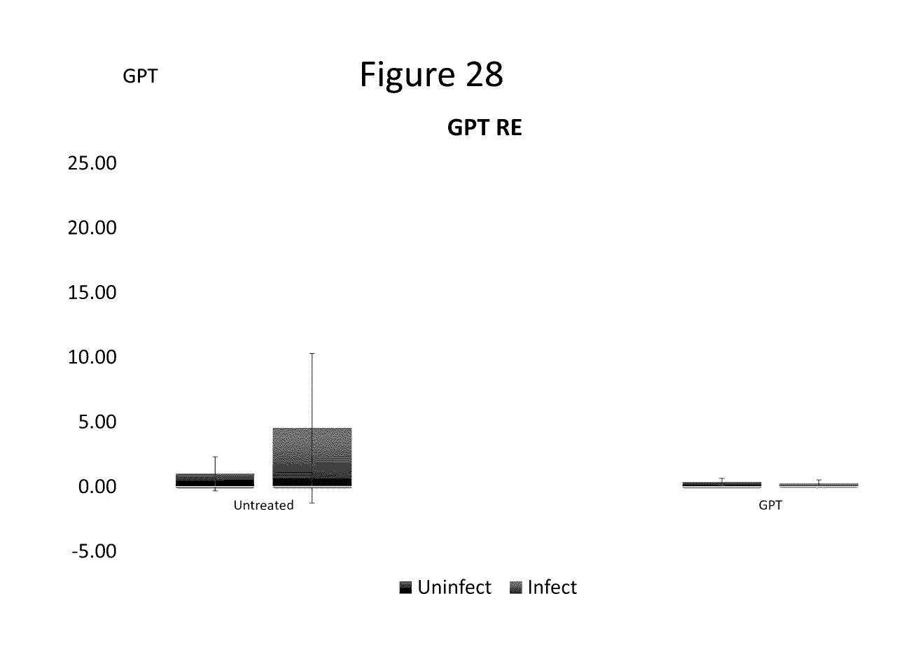

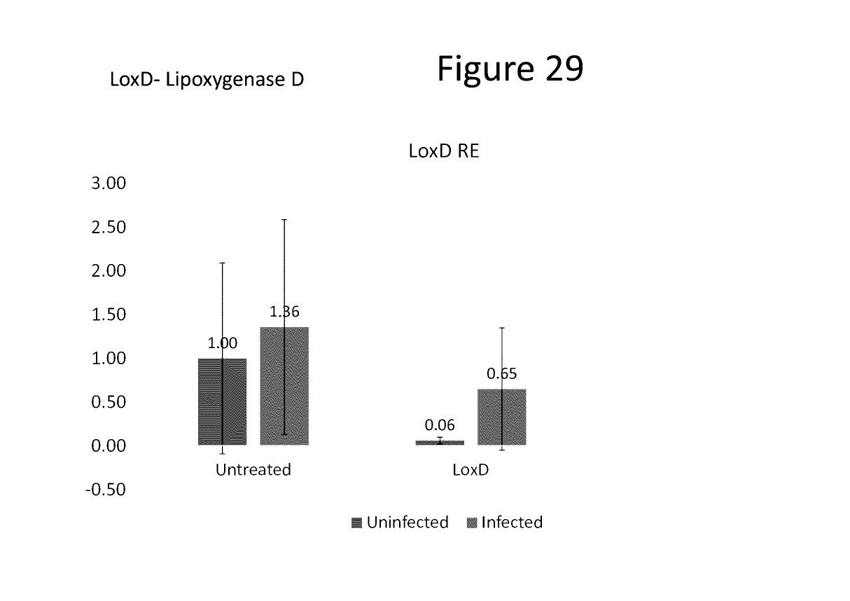

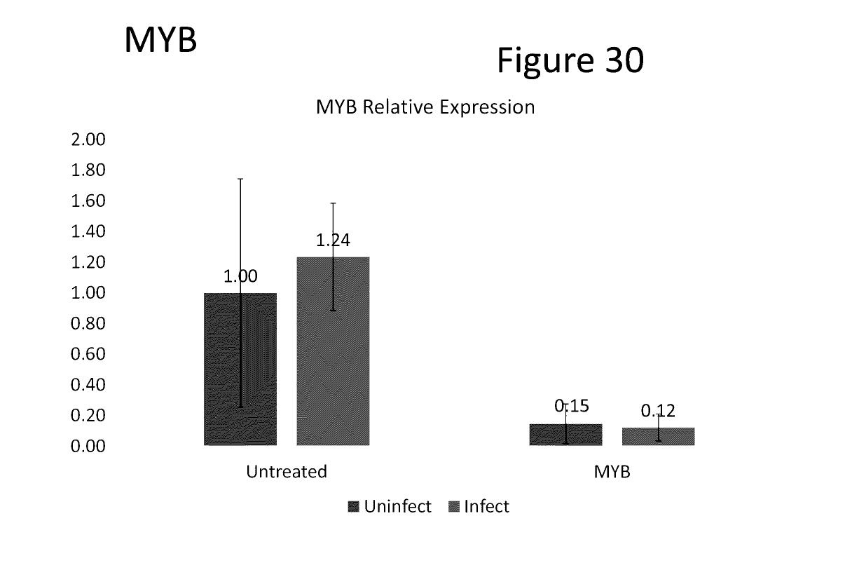

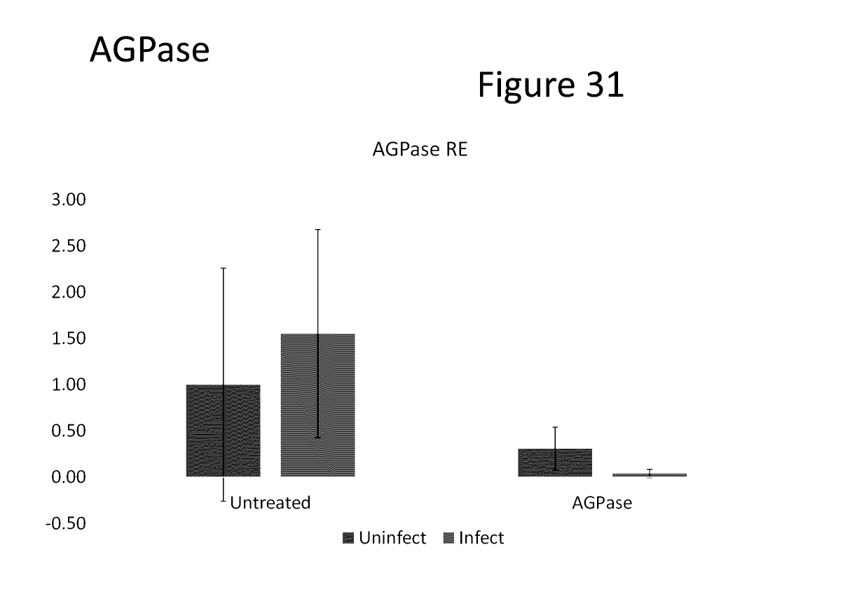

FIGS. 28-31 show examples of effective gene silencing (measured as relative expression at two weeks post Lso infection) of GPT (FIG. 28), LoxD (FIG. 29), Myb (FIG. 30) and AGPase (FIG. 31). All of the examples show increased expression of the gene in response to infection (red bars), and reduction of gene expression, at least to the levels of uninfected, untreated healthy plants, in plants treated with the VIGS gene expression silencing.

Disease Sign Index

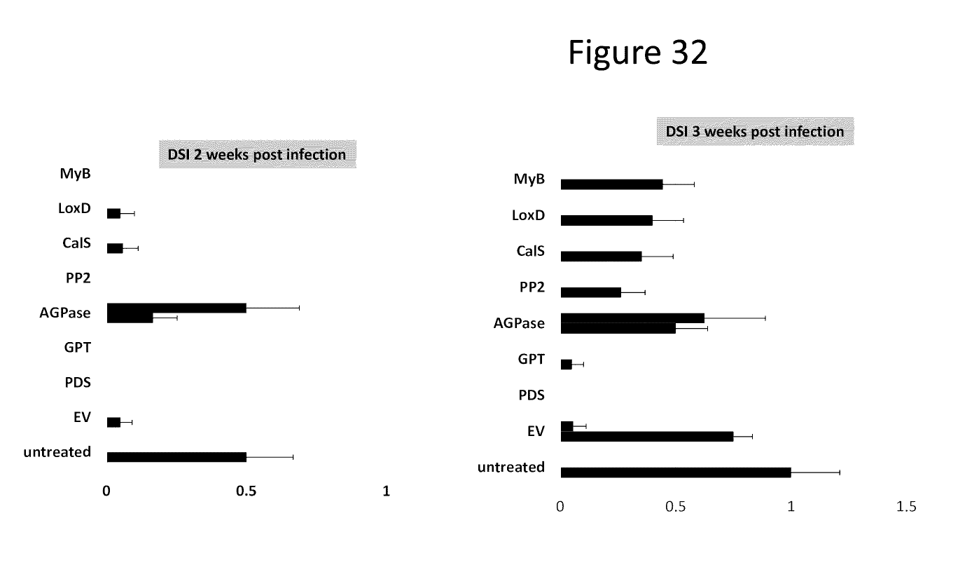

FIG. 32 shows the effect of infection, and gene silencing on the phenotype of the plants, expressed according to the semi-quantitative parameters of Disease Sign Index, recorded at 2 and 3 weeks post-Lso infection. (Note that low DSI is a superior phenotype).

Observation of the phenotypic parameters of DSI indicated good correlation between silencing of some of the candidate genes and phenotype. When compared with the untreated and empty vector-treated plants (EV) at 2 weeks, nearly all of the treated plants had significantly reduced DSI. At 3 weeks post infection, the correlation between gene silencing and low DSI persisted.

Flower Number

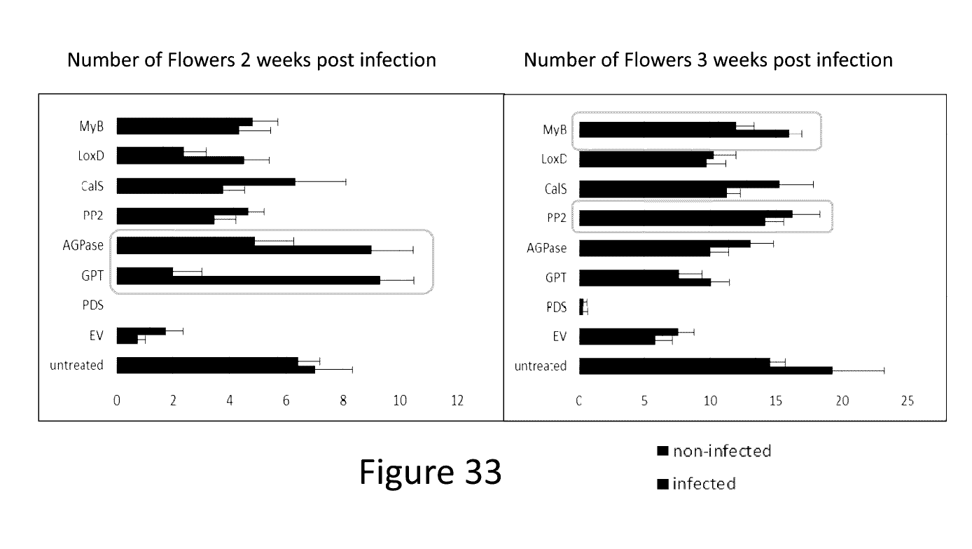

Another phenotypic parameter that can provide indication of relative health of the plant in Lso infection is the number of flowers observed. When compared with untreated and empty vector plants (EV), flower number of plants in which genes were silenced showed some advantage (FIG. 33), with all infected plants in which genes were silenced having greater flower number at 2 and 3 weeks post infection than the infected, empty vector controls. In particular, silencing of AGPase and GPT was effective in improving flower number at 2 weeks post infection, silencing of MYB and PP2 was effective at 3 weeks post infection. At 2 weeks post infection, the 2 gene groups (marked in orange box) had more flowers in infected compared to non infected plants.

Water Uptake

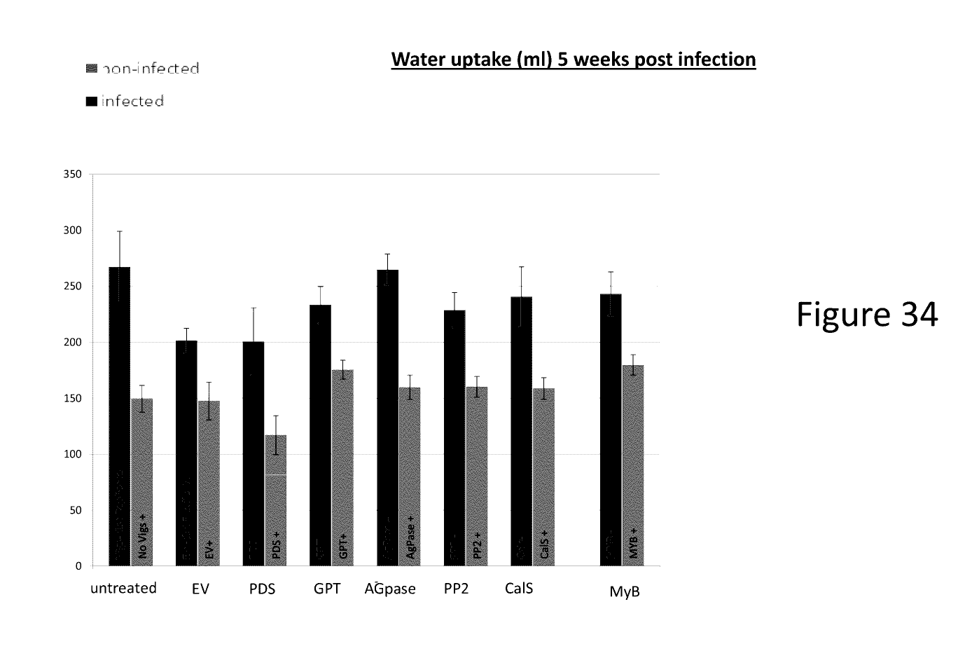

Water uptake is yet another significant phenotypic parameter which indicates relative health of the plant. Infected plants tend to imbibe less water than their non-infected counterparts (see FIG. 34, “untreated” and “EV”). Water uptake is easily measured in planter pots marked for volume in increments (for example, increments of 25 ml), by filling to the top mark (for example, 250 ml) and measuring the reduction in water volume at any given time afterwards.

FIG. 34 shows the effects of gene silencing of candidate genes on water uptake in infected plants at 5 weeks post infection. Comparison between the untreated or empty vector-treated plants and the gene-silenced plants indicates a significant improvement in water uptake with silencing of some candidate targets (e.g. GPT and MYB), and a positive trend for some others.

Taken together, the results indicate that gene silencing of some of the candidate gene targets can clearly influence the phenotype of tomato plants in response to Lso infection. Differential effects of silencing of different genes indicates that gene silencing, when effective, can affect parameters separately and at different stages of the Lso infection.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of increasing yield, growth rate, vigor, biomass, fruit quality or stress tolerance of a citrus plant when infected with a plant pathogen, the method comprising introducing into the citrus plant an isolated nucleic acid agent comprising a nucleic acid sequence which specifically reduces the expression of at least one plant pathogen resistance gene product of the plant, thereby modulating at least one plant pathogen resistance response and increasing yield, growth rate, vigor, biomass, fruit quality or stress tolerance of said citrus plant when infected with a plant pathogen.

2. A method of increasing yield, growth rate, vigor, biomass, fruit quality or stress tolerance of a plant when infected with a Candidatus Liberibacter spp, the method comprising introducing into the plant an isolated nucleic acid agent comprising a nucleic acid sequence which specifically reduces the expression of at least one plant pathogen resistance gene product of the plant, thereby modulating at least one plant pathogen resistance response and increasing yield, growth rate, vigor, biomass, fruit quality or stress tolerance of said plant when infected with a Candidatus Liberibacter spp.

3. The method of claim 1, wherein said plant pathogen is a Candidatus Liberibacter spp.

4. The method of claim 2, wherein said plant is a citrus plant or a Solanaceous plant.

5. The method of claim 2, wherein said plant is a citrus plant.

6. The method of claim 1, further comprising monitoring symptoms of infection in the infected plant following said introducing.

7-8. (canceled)

9. The method of claim 1, wherein said plant, when infected, is suffering from HuangLongBing disease (HLB or citrus greening).

10. The method of claim 1, wherein said plant pathogen resistance response is selected from the group consisting of changes in Salicylic acid levels, changes in Jasmonic acid levels, changes in Gibberellic acid levels, changes in Auxin levels, changes in Cytokinin levels, changes in Ethylene levels, changes in ABA phyto-hormones levels, up-regulation of phyto-hormone-related genes, biosynthesis, deposition and degradation of callose, reactive oxygen species production, functional FLS2/BAK1 complex formation, protein-kinase pathway activation, phloem blockage, starch accumulation, starch accumulation in phloem parenchyma cells, polymer sieve formation, changes in carbohydrate metabolism, cambial activity aberrations, sucrose accumulation and carotenoid synthesis.

11. The method of claim 1, wherein said increase in yield, growth rate, vigor, biomass, fruit quality or stress tolerance is a change in a parameter selected from the group consisting of increased water uptake, increased plant height, increased plant flower number, decreased starch accumulation and decreased Disease Sign Index.

12-13. (canceled)

14. The method of claim 1, wherein said plant pathogen resistance gene product is selected from plant gene products having upregulated expression following infection of the plant with said plant pathogen.

15. The method of claim 1, wherein said plant pathogen resistance gene product is selected from the group consisting of the polynucleotide sequences of Table IV and/or Table IV(a).

16. The method of claim 2, wherein said plant pathogen resistance gene product is selected from the group consisting of plant gene products having at least 60% identity to any of the polynucleotide sequences of Table II.

17. The method of claim 15, wherein said plant pathogen resistance gene product is selected from the group consisting of the polynucleotide sequences of Table V.

18. The method of claim 16, wherein said plant pathogen resistance gene product is selected from the group consisting of plant gene products having at least 60% identity to any of the polynucleotide sequences of Table III.

19. The method of claim 1, wherein said plant pathogen resistance gene product is selected from the group consisting of an ADP-glucose pyrophosphorylase large subunit (AGPase) gene product, a glucose-6-phosphate/phosphate translocator (GPT) gene product, a Callose synthase gene product and a Myb transcriptional regulator (MYB) gene product.

20. (canceled)

21. The method of claim 2, wherein said plant is a fruit tree, and optionally, a citrus tree.

22. The method of claim 1, wherein said isolated nucleic acid agent further comprises a cell penetrating agent.

23. The method of claim 1, wherein said introducing is following detection of a symptom of infection of said plant with said pathogen.

24. An isolated nucleic acid agent comprising a nucleic acid sequence which specifically reduces the expression of at least one plant pathogen resistance gene product selected from the group consisting of plant gene products having at least 60% identity to any of the polynucleotide sequences of Table II.

25-30. (canceled)

31. The isolated nucleic acid agent of claim 24, wherein said plant pathogen resistance gene is a HuangLongBing-associated plant pathogen resistance gene selected from SEQ ID NOs: 204-265 and 489-516 or homologs thereof.

32. The isolated nucleic acid agent of claim 24, wherein said plant pathogen resistance gene is a HuangLongBing-associated plant pathogen resistance gene selected from SEQ ID NOs: 1-203 or homologs thereof.

33. The isolated nucleic acid agent of claim 24, wherein said plant pathogen resistance gene is selected from SEQ ID NOs: 623-714 or homologs thereof.

34. The isolated nucleic acid agent of claim 24, wherein said plant pathogen resistance gene product is selected from the group consisting of an ADP-glucose pyrophosphorylase large subunit (AGPase) gene product, a glucose-6-phosphate/phosphate translocator (GPT) gene product, a Callose synthase gene product and a Myb transcriptional regulator (MYB) gene product.

35. The isolated nucleic acid agent of claim 24, wherein said nucleic acid sequence comprises a nucleic acid sequence selected from the group consisting of SEQ ID NOs. 528, 530, 532 and 536.

36. A nucleic acid construct comprising a nucleic acid sequence encoding the isolated nucleic acid agent of claim 24.

37-41. (canceled)

42. A citrus plant comprising at least one exogenous isolated nucleic acid agent comprising a nucleic acid sequence which specifically reduces the expression of at least one plant pathogen resistance gene product.

43. A plant comprising at least one exogenous isolated nucleic acid agent comprising a nucleic acid sequence which specifically reduces the expression of at least one plant pathogen resistance gene product selected from the group consisting of plant gene products having at least 60% identity to any of the polynucleotide sequences of Table II.

44. The plant of claim 43, wherein said plant is selected from the group consisting of a tree, a shrub, a bush, a seedling, a scion, a rootstock, an inarched plant, a bud, a budwood, a root and a graft.

45. The plant of claim 43, wherein said plant is a citrus or citrus-related plant.

46. The plant of claim 42, wherein said plant is a plant at risk of infection with CaLas.

47. A cell of the plant of claim 43.

48-50. (canceled)

51. The method of claim 2, further comprising monitoring symptoms of infection in the infected plant following said introducing.

52. The method of claim 2, wherein said plant pathogen resistance gene product is selected from the group consisting of an ADP-glucose pyrophosphorylase large subunit (AGPase) gene product, a glucose-6-phosphate/phosphate translocator (GPT) gene product, a Callose synthase gene product and a Myb transcriptional regulator (MYB) gene product.
